(19) United States
(12) Patent Application Publication
(10) Pub. No.: US 2016/0298192 A1
(43) Pub. Date: Oct. 13, 2016

(54) METHODS AND KITS FOR IDENTIFYING SUSCEPTIBILITY TO ANTIRESORPTIVE-AGENT-INDUCED OSTEONECROSIS OF THE JAW

(71) Applicant: MICROMEDIC TECHNOLOGIES LTD., Tel Aviv (IL)

(72) Inventors: Adi ELKELES, Tel Mond (IL); Vered Vivi ZIV, Mazkeret Batya (IL); Noa DAVIS, Tel Aviv (IL)

(21) Appl. No.: 15/038,380

(22) PCT Filed: Dec. 4, 2014
(86) PCT No.: PCT/IL2014/051051

§ 371 Date: May 20, 2016

Related U.S. Application Data
(60) Provisional application No. 61/973,872, filed on Apr. 2, 2014.

(30) Foreign Application Priority Data
Dec. 4, 2013 (IL) 229793

(51) Int. Cl. C12Q 1/68; A61K 31/663; A61K 31/675

(52) U.S. Cl. CPC C12Q 1/6883; A61K 31/675; A61K 31/663; C12Q 2600/156

(57) ABSTRACT

The present invention relates, according to some embodiments, to methods and kits for identifying susceptibility to osteonecrosis of the jaw (ONJ), including, antiresorptive-agent-induced jaw osteonecrosis (ARONJ), in a subject in need thereof. The present invention further relates to method and kits for preventing the development of ONJ, inhibiting the progression of ONJ, or reducing the risk to develop ONJ in subjects in need thereof. The present invention further provides methods and kits for treating ONJ.

### FIELD OF THE INVENTION

The present invention relates, according to some embodiments, to methods and kits for identifying susceptibility to osteonecrosis of the jaw (ONJ), including, anti-resorptive-agent-induced jaw osteonecrosis (ARONJ), in a subject in need thereof. The present invention further relates to method and kits for preventing the development of ONJ, inhibiting the progression of ONJ, or reducing the risk to develop ONJ in subjects in need thereof. The present invention further provides methods and kits for treating ONJ.

### BACKGROUND OF THE INVENTION

Bone anti-resorptive agents are a class of drugs that decrease or prevent bone resorption processes, for example by inhibiting the activities and functions of osteoclasts (bone resorbing cells) and perturbing the differentiation of osteoblasts (bone forming cells). Typically, bone anti-resorptive agents are prescribed to alleviate bone pain, bone destruction and hypercalcemia in many cancer patients.

Bisphosphonates are amongst the most commonly used bone-anti-resorptive agents. In millions of postmenopausal women bisphosphonates are commonly prescribed to stabilize bone loss caused by osteoporosis. For this purpose, oral bisphosphonates such as etidronate, risedronate, tiludronate and alendrtonate are prescribed to patients with osteoporosis. More potent bisphosphonates are indicated for stabilizing metastatic cancer (primarily breast and prostate) deposits in bone, treating bone resorption defects of multiple myeloma and correcting severe hypercalcemia. Currently, bisphosphonates are administered intravenously and include, for example, pamidronate disodium and zoledronic acid.

Recent reports suggest an association between the use of bone anti-resorptive agents, such as, bisphosphonates and osteonecrosis of the jaw. Anti-resorptive-agent-induced osteonecrosis of the jaw (ARONJ) is a morbid bone disorder occurring in a subset of patients who are treated with bone anti-resorptive agents. ARONJ which arises from treatment with bisphosphonates is referred to as bisphosphonate-induced osteonecrosis of the jaw (BONJ or BRONJ). During the disease, necrotic jaw bone becomes exposed and fails to heal. However, the pathophysiological mechanisms by which this complication manifests in users of bone anti-resorptive agents are still unknown.

The majority of BONJ cases seen and reported were patients treated with IV bisphosphonates, but cases have also been reported in association with oral bisphosphonates. While up to 13% of patients receiving IV bisphosphonates develop BONJ, estimates for oral bisphosphonates are 1:10,000 to 1:100,000. Although it is not possible to conduct a controlled, randomized, prospective, blinded study to prove the specific causal relationship between bisphosphonate therapy and exposed bone, the drugs pamidronate disodium, zoledronic acid, and more rarely alendronate sodium, have shown a direct correlation.

Most BONJ cases to date are diagnosed in cancer patients with bone metastases. However, BONJ cases have been also reported after oral therapy for osteoporosis. Thus, a large proportion of the general population (e.g., post-menopausal women and cancer patients) may be at risk.

WO2010/005939 and WO2011/084387 disclose methods for determining the pharmacogenetic, pharmacokinetic and cellular basis of BONJ via associating particular proteins and particular single nucleotide polymorphisms with a risk for developing BONJ after receiving bisphosphonate treatment.

WO2012/138745 discloses methods and compositions for identifying individuals having an increased risk of developing ARONJ. These applications disclose the association of Single Nucleotide Polymorphisms (SNPs) in the RNA-binding motif, single-stranded-interacting protein 3 (RBMS3) gene with osteonecrosis of the jaw among bisphosphonate users, as well as SNPs in other genes, including insulin-like growth factor I receptor (IGF1R), insulin-like growth factor binding protein 7 (IGFBP7), dihydropyrimidine dehydrogenase (DPYD), ATP-binding cassette, sub-family C (CFTR/MRP), member 4 (ABCC4), and glutathione S-transferase mu-2 (GSTM2).

Mucin-4 is a high molecular weight glycoprotein encoded by the mucin-4 (MUC4) gene. Mucin-4 is composed of two subunits, alpha and beta, both transcribed from the mucin-4 gene. Over 24 splice variants have been found for MUC4, in normal as well as abnormal tissue, some soluble and some membrane bound. Many polymorphisms are observed in the tandem repeat region of the alpha subunit of MUC4, which has a variable number of repeats. Polymorphisms in MUC4 have recently been shown to be associated with endometriosis development and endometriosis-related infertility (Chang et al., 2011, BMC Medicine, 9:19).

There is still an unmet need for methods for preventing, inhibiting or attenuating the development of ONJ, and specifically, ARONJ. The foregoing examples of the related art and limitations related therewith are intended to be illustrative and not exclusive. Other limitations of the related art will become apparent to those of skill in the art upon a reading of the specification and a study of the figures.

### SUMMARY OF THE INVENTION

The present invention provides, according to some embodiments, methods for determining whether a subject is predisposed for ARONJ by performing genetic profiling on a sample from said subject. According to some embodiments, genetic profiling according to the invention comprises determining the presence of at least one single nucleotide polymorphism (SNP) within the gene Mucin-4 (MUC4), as set forth in SEQ ID NO: 48. According to some embodiments, said at least one SNP is within the genomic region coding for Exon 2 of MUC4 transcript 1, said Exon being found between chromosomal positions 195505661-195518368 of chromosome 3, as set forth in SEQ ID NO: 49. According to some embodiments, the at least one SNP is within chromosomal positions 195505883-195515290 of chromosome 3, as set forth in SEQ ID NO: 50.

According to some embodiments, the presence of said at least one single nucleotide polymorphisms in MUC4 within a sample obtained from the subject is indicative of predisposition of said subject to develop anti-resorptive-agent-induced jaw osteonecrosis (ARONJ) following therapy with bone anti-resorptive agents.

According to some embodiments, the presence of said at least one single nucleotide polymorphisms in MUC4 within a sample obtained from the subject is indicative of predisposition of said subject to develop osteonecrosis of the jaw (ONJ). According to other embodiments, the presence of said at least one single nucleotide polymorphisms in MUC4 within a sample obtained from the subject is indicative of predisposition of said subject to develop bisphosphonate-induced jaw osteonecrosis (BONJ) following bisphosphonate therapy.

The terms “anti-resorptive-agent-induced osteonecrosis of the jaw” and “ARONJ” as used herein are interchangeable with any one of the terms “bisphosphonate-induced osteonecrosis of the jaw”, “BONJ”, “BRONJ” and/or the terms osteonecrosis of the jaw and “ONJ”.

According to additional embodiments, the present invention provides a method of treating a subject predisposed for ARONJ by administering to said subject a treatment having a low probability of inducing ARONJ. According to some embodiments, the subject is afflicted with a condition which may benefit from administration of bone anti-resorptive agent, by administration of a treatment having a low probability of inducing ARONJ in the subject. According to some embodiments, a treatment having a low probability of inducing ARONJ is selected from the group consisting of: a bisphosphonate not associated with bisphosphonate-induced jaw osteonecrosis (BONJ), a bisphosphonate having a low probability of inducing BONJ, bisphosphonate at a low dosage, bisphosphonate at a low administration frequency, a non-bisphosphonate anti-resorptive agent having a low probability of inducing ARONJ, a non-bisphosphonate anti-resorptive agent not associated with ARONJ or a combination thereof. Each possibility represents a separate embodiment of the present invention.

According to some embodiments, treatment further includes adequate regular dental care and maintenance of good oral hygiene.

According to other embodiments, treatment further includes cessation periods of treatment with an anti-resorptive agent for at least 3 months prior to and/or following an invasive dental surgery.

The present invention is based in part on the unexpected discovery of novel SNPs within Mucin-4 gene, which were shown, for the first time, to be associated with ARONJ predisposition. As exemplified herein below, SNPs within Mucin-4 gene have a significantly higher occurrence in bisphosphonate-treated patients which developed BONJ in comparison with bisphosphonate-treated patients lacking BONJ symptoms.

According to one aspect, the present invention provides a method of treating a disease or disorder requiring anti-resorptive therapy, the method comprising:

determining the presence of at least one single nucleotide polymorphism (SNP) in a sample obtained from a subject having a disease or disorder requiring anti-resorptive therapy, wherein said at least one single nucleotide polymorphism is within the Mucin-4 gene;

identifying a subject having said at least one single nucleotide polymorphism; and

administering to said subject a composition comprising at least one anti-resorptive agent not associated with ARONJ or having a low probability of inducing ARONJ.

According to some embodiments, said at least one SNP is within SEQ ID NO: 48, corresponding to the entire MUC4 gene.

According to some embodiments, said at least one SNP is within SEQ ID NO: 49, corresponding to Exon 2 of the MUC4 gene, positions 195505661-195518368 on Chromosome 3.

According to some embodiments, said at least one SNP is within SEQ ID NO: 50, corresponding to a fraction of Exon 2 in the MUC4 gene, positions 195505883-195515290 on Chromosome 3.

According to some embodiments, said at least one SNP is within SEQ ID NO: 56, corresponding to a fraction of Exon 2 in the MUC4 gene positions 195505884-195508789.

According to some embodiments, said at least one SNP is within SEQ ID NO: 57, corresponding to a fraction of Exon 2 in the MUC4 gene positions 195513831-195515290.

According to some embodiments, said at least one SNP is selected from the SNPs listed in Tables 2, 3 and 5.

According to some embodiments, said at least one SNP is selected from the group consisting of:

C at position 195505883 of Chromosome 3 (hereinafter “SNP No. 101”);

G at position 195505886 of Chromosome 3 (hereinafter “SNP No. 102”);

C at position 195506091 of Chromosome 3 (hereinafter “SNP No. 103”);

G at position 195506118 of Chromosome 3 (hereinafter “SNP No. 104”);

G at position 195506137 of Chromosome 3 (hereinafter “SNP No. 105”);

C at position 195506342 of Chromosome 3 (hereinafter “SNP No. 106”);

T at position 195506531 of Chromosome 3 (hereinafter “SNP No. 107”);

A at position 195506953 of Chromosome 3 (hereinafter “SNP No. 108”);

T at position 195506963 of Chromosome 3 (hereinafter “SNP No. 109”);

T at position 195506966 of Chromosome 3 (hereinafter “SNP No. 110”);

A at position 195506974 of Chromosome 3 (hereinafter “SNP No. 111”);

A at position 195507433 of Chromosome 3 (hereinafter “SNP No. 112”);

G at position 195507443 of Chromosome 3 (hereinafter “SNP No. 113”);

A at position 195507445 of Chromosome 3 (hereinafter “SNP No. 114”);

T at position 195507446 of Chromosome 3 (hereinafter “SNP No. 115”);

G at position 195507731 of Chromosome 3 (hereinafter “SNP No. 116”);

T at position 195507771 of Chromosome 3 (hereinafter “SNP No. 117”);

G at position 195507943 of Chromosome 3 (hereinafter “SNP No. 118”);

T at position 195507999 of Chromosome 3 (hereinafter “SNP No. 119”);

G at position 195508005 of Chromosome 3 (hereinafter “SNP No. 120”);

T at position 195508021 of Chromosome 3 (hereinafter “SNP No. 121”);

G at position 195508418 of Chromosome 3 (hereinafter “SNP No. 122”);

T at position 195508451 of Chromosome 3 (hereinafter “SNP No. 123”);

T at position 195508453 of Chromosome 3 (hereinafter “SNP No. 124”);

C at position 195508454 of Chromosome 3 (hereinafter “SNP No. 125”);

A at position 195508462 of Chromosome 3 (hereinafter “SNP No. 126”);

T at position 195508475 of Chromosome 3 (hereinafter “SNP No. 127”);

C at position 195508478 of Chromosome 3 (hereinafter “SNP No. 128”);

C at position 195508501 of Chromosome 3 (hereinafter “SNP No. 129”);

T at position 195508502 of Chromosome 3 (hereinafter “SNP No. 130”);

A at position 195508702 of Chromosome 3 (hereinafter “SNP No. 131”);

G at position 195508709 of Chromosome 3 (hereinafter “SNP No. 132”);

G at position 195508716 of Chromosome 3 (hereinafter “SNP No. 133”);

T at position 195508722 of Chromosome 3 (hereinafter “SNP No. 134”);

G at position 195508758 of Chromosome 3 (hereinafter “SNP No. 135”);

G at position 195508777 of Chromosome 3 (hereinafter “SNP No. 136”);

G at position 195508786 of Chromosome 3 (hereinafter “SNP No. 137”);

T at position 195508789 of Chromosome 3 (hereinafter “SNP No. 138”);

C at position 195508790 of Chromosome 3 (hereinafter “SNP No. 139”);

G at position 195511534 of Chromosome 3 (hereinafter “SNP No. 140”);

A at position 195512567 of Chromosome 3 (hereinafter “SNP No. 141”);

G at position 195513831 of Chromosome 3 (hereinafter “SNP No. 142”);

G at position 195513846 of Chromosome 3 (hereinafter “SNP No. 143”);

G at position 195513847 of Chromosome 3 (hereinafter “SNP No. 144”);

A at position 195514450 of Chromosome 3 (hereinafter “SNP No. 145”);

G at position 195515113 of Chromosome 3 (hereinafter “SNP No. 146”);

G at position 195515290 of Chromosome 3 (hereinafter “SNP No. 147”);

G at position 195515271 of Chromosome 3 (hereinafter “SNP No. 151”);

A at position 195474159 of Chromosome 3 (hereinafter “SNP No. 152”);

T at position 195507925 of Chromosome 3 (hereinafter “SNP No. 153”);

A at position 195513413 of Chromosome 3 (hereinafter “SNP No. 154”);

G at position 195511534 of Chromosome 3 (hereinafter “SNP No. 155”); and

A at position 195511534 of Chromosome 3 (hereinafter “SNP No. 156”).

With respect to SNP Nos. 155 and 156, these SNPs are mutations at the same point on exon 2 of MUC4, namely, Chr3:195511534. At this point the major allele (reference allele or wild type) is T where the altered allele in some BRONJ patients is G (SNP NO. 155) and in others A (SNP NO. 156).

According to some embodiments, the at least one bone anti-resorptive agent not associated with ARONJ or having a low probability of inducing ARONJ is selected from the group consisting of: bisphosphonate not associated with BONJ, bisphosphonate having a low probability of inducing BONJ, bisphosphonate at a low dosage, a non-bisphosphonate anti-resorptive agent having a low probability of inducing ARONJ, a non-bisphosphonate anti-resorptive agent not associated with ARONJ and a combination thereof. Each possibility represents a separate embodiment of the present invention.

According to some embodiments, the at least one anti-resorptive agent having a low probability of inducing ARONJ is selected from the group consisting of: a bisphosphonate having a low probability of inducing BONJ, a non-bisphosphonate anti-resorptive agent having a low probability of inducing ARONJ, a non-bisphosphonate anti-resorptive agent not associated with ARONJ or a combination thereof. Each possibility represents a separate embodiment of the present invention.

According to another embodiment, an anti resorptive agent having a low probability of inducing ARONJ, is any one or more of risedronate (such as, Actonel®) and ibandronate (such as, Boniva®).

According to some embodiments, the method of treating a subject afflicted with a condition which may benefit from anti-resorptive therapy further comprises providing said sample from a subject.

According to some embodiments, treating a disease or disorder requiring anti-resorptive therapy according to the present invention further comprises at least one treatment selected from the group consisting of: preventing ARONJ development, inhibiting ARONJ development, suppressing ARONJ development and attenuating ARONJ manifestation. Each possibility represents a separate embodiment of the present invention.

According to some embodiments, the disease or disorder requiring bisphosphonate therapy is selected from the group consisting of: osteoporosis, osteitis deformans, osteogenesis imperfecta, bone metastasis, breast cancer, prostate cancer, lung cancer and multiple myeloma. Each possibility represents a separate embodiment of the present invention.

According to some embodiments, the bone metastasis is associated with a malignant disease, such as, breast cancer, prostate cancer and lung cancer.

It is to be understood, that the sample is any sample obtained from a subject which includes DNA and/or mRNA, and thus is by no means limited to any specific sample.

According to some embodiments, the sample comprises blood, serum, plasma, urine, sweat, buccal smear or saliva. Each possibility represents a separate embodiment of the present invention.

According to some embodiments, the at least one single nucleotide polymorphism comprises a plurality of single nucleotide polymorphisms. According to some embodiments, the at least one single nucleotide polymorphism comprises at least 2 single nucleotide polymorphisms. According to some embodiments, the at least one single nucleotide polymorphism comprises at least 5 single nucleotide polymorphisms. According to some embodiments, the at least one single nucleotide polymorphism comprises at least 10 single nucleotide polymorphisms.

According to some embodiments, the at least one single nucleotide polymorphism is a polymorphism present in subjects predisposed for ARONJ and not in healthy subjects with a significance of P≦0.1. According to some embodiments, the at least one single nucleotide polymorphism is a polymorphism present in subjects predisposed for BONJ and not in healthy subjects with a significance of P<0.05.

According to some embodiments, determining the presence of said at least one single nucleotide polymorphism comprises identifying the at least one single nucleotide polymorphism in at least one gene product of the Mucin-4 gene as set forth in SEQ ID NO: 48.

As used herein, the term analyzing a gene or gene product refers to identifying the at least one single nucleotide polymorphism in the gene or gene product, respectively.

According to some embodiments, the at least one gene product is an mRNA molecule.

According to some embodiments, analyzing the at least one gene product of the Mucin-4 gene is by specific hybridization of a polynucleotide to said mRNA molecule.

According to some embodiments, the polynucleotide for hybridization is selected from SEQ ID NOs: 1-47 and 51-55.

According to some embodiments, the at least one gene product is a protein. According to some embodiments, analyzing the at least one gene product of the Mucin-4 gene is by specific binding of an antibody to said protein.

According to some embodiments, the at least one anti-resorptive agent not associated with ARONJ or having a low probability of inducing ARONJ is a bisphosphonate not associated with bisphosphonate-induced jaw osteonecrosis (BONJ). According to some embodiments, the bisphosphonate having a low probability of inducing BONJ is selected from the group consisting of: clondronate, etidronate, tiludronate, risedronate, ibandronate and a combination thereof. Each possibility represents a separate embodiment of the present invention.

According to some embodiments, the non-bisphosphonate anti-resorptive agent not associated with ARONJ is an inhibitor of cathepsin K. According to some embodiments, the inhibitor of cathepsin K is Odanacatib (N-(1-cyanocyclopropyl)-4-fluoro-N²-{(1S)-2,2,2-trifluoro-1-[4′-(methylsulfonyl)biphenyl-4-yl]ethyl}-L-leucinamide).

According to some embodiments, the non-bisphosphonate anti-resorptive agent having a low probability of inducing ARONJ is selected from the group consisting of: denosumab, estrogen, an estrogen agonist, an estrogen antagonist and a combination thereof. Each possibility represents a separate embodiment of the present invention.

According to non-limiting examples, agents which modulate the estrogen pathway such as raloxifene and tamoxifen may be used as anti-resorptive agents having a low probability of inducing ARONJ according to the present invention.

According to some embodiments, administering a composition comprising at least one anti-resorptive agent not associated with ARONJ or having a low probability of inducing ARONJ comprises administration of said composition at a low administration frequency. According to some embodiments, the low administration frequency is administration less than 12 times a year.

According to another aspect, the present invention provides a method of identifying a subject having a predisposition to anti-resorptive-agent-induced jaw osteonecrosis (ARONJ) following administration of an anti-resorptive agent, the method comprising the steps of: determining, in a sample derived from said subject, the presence of at least one single nucleotide polymorphism within the Mucin-4 gene, wherein the presence of said at least one single nucleotide polymorphism within said sample is indicative of predisposition of said subject to develop ARONJ.

According to some embodiments, said at least one SNP is within SEQ ID NO: 48.

According to some embodiments, said at least one SNP is within SEQ ID NO: 49.

According to some embodiments, said at least one SNP is within SEQ ID NO: 50.

According to some embodiments, said at least one SNP is within SEQ ID NO: 56.

According to some embodiments, said at least one SNP is within SEQ ID NO: 57.

According to some embodiments, said at least one SNP is selected from the SNPs listed in Tables 2, 3 and 5.

According to some embodiments, said at least one SNP is selected from SNP NOs: 101-147 and 151-156. Each possibility represents a separate embodiment of the present invention.

According to some embodiments, the method of identifying a subject having a predisposition to anti-resorptive agent-induced jaw osteonecrosis (ARONJ) further comprises obtaining said sample from said subject.

According to some non-limiting embodiments, determining in the sample derived from said subject the presence of the least one single nucleotide polymorphism is facilitated using a method selected from the group consisting of: sequencing, Restriction Fragment Length Polymorphism (RFLP), DNA Microarray, SNP Microarray, Polymerase Chain Reaction (PCR), Reverse-Transcription Polymerase Chain Reaction (RT-PCR), denaturing HPLC and gel electrophoresis, Single Strand Conformation Polymorphism and gel electrophoresis, DNA hybridization analysis Mass Spectrometry and a combination thereof. Each possibility represents a separate embodiment of the present invention.

According to yet another aspect, the present invention provides a kit for determining predisposition to anti-resorptive agent-induced jaw osteonecrosis (ARONJ) following administration of an anti-resorptive agent, the kit comprising: at least one biological probe configured to specifically hybridize to a biomarker; wherein said biomarker is the Mucin-4 gene or a fragment thereof, a gene product of the Mucin-4 gene and cDNA corresponding to the Mucin-4 gene, comprising at least one single nucleotide polymorphism; and at least one detection reagent.

According to some embodiments, the biological probe is selected from a nucleic acid, a peptide and a combination thereof.

According to some embodiments, the biological probe is selected from a nucleic acid, a peptide, a polypeptide, an antibody and a combination thereof. Each possibility represents a separate embodiment of the present invention.

According to some embodiments, the biological probe is a nucleic acid selected from a DNA probe and an RNA probe. Each possibility represents a separate embodiment of the present invention.

According to some embodiments, the biological probe is an antibody.

According to some embodiments, the gene product is selected from a nucleic acid, a peptide and a combination thereof. Each possibility represents a separate embodiment of the present invention.

According to yet another aspect, the present invention provides a method of preventing anti-resorptive-agent-induced jaw osteonecrosis (ARONJ) in a subject having a disease or disorder requiring bisphosphonate therapy, the method comprising:

determining the presence of at least one single nucleotide polymorphism (SNP) within a sample obtained from said subject, wherein said SNP is within the Mucin-4 gene; and

administering to said subject a composition comprising at least one anti-resorptive agent not associated with ARONJ or having a low probability of inducing ARONJ.

According to yet another aspect, the present invention provides use of at least one anti-resorptive agent not associated with ARONJ or having a low probability of inducing ARONJ for treating or preventing anti-resorptive-agent-induced jaw osteonecrosis (ARONJ) in a subject having a disease or disorder requiring bisphosphonate therapy, wherein said subject is identified as having at least one single nucleotide polymorphism (SNP) within the Mucin-4 gene.

Further embodiments, features, advantages and the full scope of applicability of the present invention will become apparent from the detailed description and drawings given hereinafter. However, it should be understood that the detailed description, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

Exemplary embodiments are illustrated in referenced figures. Dimensions of components and features shown in the figures are generally chosen for convenience and clarity of presentation and are not necessarily shown to scale. It is intended that the embodiments and figures disclosed herein are to be considered illustrative rather than restrictive. The figures are listed below.

FIG. 1 presents the distribution of the 42 SNPs set forth in Table 2 in bisphosphonate-treated subjects which developed BONJ: white background—homozygous presence of a wild-type allele (0/0), grey background—heterozygous presence of an allele containing a SNP (0/1), and black background—homozygous presence of an allele containing a SNP (1/1).

FIG. 2 presents the distribution of the 42 SNPs as set forth in Table 2 in bisphosphonate-treated subjects lacking BONJ symptoms (same color coding as in FIG. 1).

FIG. 3 presents the distribution of the 47 SNPs as set forth in Tables 2 and 3 in bisphosphonate-treated subjects which developed BONJ (same color coding as in FIG. 1).

FIG. 4 is a table depicting the distribution of the 47 SNPs as set forth in Tables 2 and 3 in bisphosphonate-treated subjects lacking BONJ symptoms (same color coding as in FIG. 1).

FIG. 5 is a dot plot depicting non-synonymous SNPs along the MUC4 gene correlated with their significance of occurrence in bisphosphonate-treated subjects which developed BONJ as compared to occurrence in bisphosphonate-treated subjects lacking BONJ symptoms (Ser;Thr-The SNP is in a Ser/Thr residue, Pro-The SNP is in a Pro residue). The dotted line marks the threshold for statistical significance (p-value<0.01).

### DETAILED DESCRIPTION OF THE INVENTION

The present invention provides a method of identifying a subject having a predisposition to ARONJ, comprising the step of: identifying, in a sample derived from a subject, the presence of at least one single nucleotide polymorphism in the Mucin-4 gene, gene product or a fragment thereof, thereby determining predisposition of said subject to develop ARONJ.

As used herein, the Mucin-4 (MUC4) gene refers to the sequence of the wild-type human Mucin4 gene, as set forth in SEQ ID NO: 48, present at positions 195473636-195539148 of chromosome 3. It is to be understood that chromosomal positions referred to herein relate to chromosomal positions within the Mucin-4 gene on human chromosome 3.

According to some embodiments, the at least one SNP differentiates between a subject predisposed to develop ARONJ following administration of an anti-resorptive agent and a subject which is not predisposed to develop ARONJ following administration of an anti-resorptive agent. According to some embodiments, the at least one SNP differentiates between a subject predisposed to develop ARONJ following administration of an anti-resorptive agent and a subject which is not predisposed to develop ARONJ following administration of an anti-resorptive agent with a significance of at least P≦0.1; at least P<0.05 or at least P<0.01. Each possibility represents a separate embodiment of the present invention.

According to some embodiments, the at least one SNP is present in subjects predisposed for ARONJ following administration of an anti-resorptive agent and not-present in subjects which are not predisposed for ARONJ following administration of an anti-resorptive agent.

According to other embodiments, the at least one SNP is having a significantly higher probability of being present in subjects predisposed for ARONJ following administration of an anti-resorptive agent than in subjects that are not predisposed for ARONJ following administration of an anti-resorptive agent.

According to some embodiments, the at least SNP is present in subjects predisposed for ARONJ and not in healthy subjects with a significance of at least P≦0.1; at least P<0.05 or at least P<0.01. Each possibility represents a separate embodiment of the present invention.

According to some embodiments, significance values referred to herein are values calculated by the Fisher's exact test.

According to some embodiments, the SNPs disclosed herein refer to SNPs within genomic regions coding for exons of Mucin-4 transcript 1, as referred to by accession number ENST00000463781 in the ENSEMBL genome database. According to some embodiments, the SNPs disclosed herein refer to SNPs within exons of mRNA molecules encoded by the Mucin-4 gene. According to some embodiments, the SNPs disclosed herein refer to SNPs within the genomic region encoding Exon2 of Mucin-4 mRNA transcript 1, the coding region for said exon spanning positions 195505661-195518368 of chromosome 3, as set forth in SEQ ID NO: 49. Exon 2 of the Mucin-4 gene as set forth in SEQ ID NO: 49 refers to the sequence as represented by accession number ENSE00001854802 in the ENSEMBL genome database. According to some embodiments, the SNPs disclosed herein refer to SNPs within the genomic region encoding part of Exon2 of Mucin-4 mRNA transcript 1, the genomic regions spanning positions 195505883-195515290 of chromosome 3, as set forth in SEQ ID NO: 50.

The term “wild-type” refers to naturally-occurring, e.g. naturally-occurring polynucleotide or peptide. According to some embodiments, naturally occurring refers to occurrence in the majority of the population. According to certain embodiments, naturally occurring refers to occurrence in subjects which are not predisposed for BONJ.

As used herein, the term “gene” refers to a linear sequence of nucleotides along a segment of DNA which provides the coded instructions for synthesis of an RNA molecule and may further determine the amino-acid sequence of a peptide encoded by said RNA.

The terms “polymorphic site” or “polymorphism”, as used herein, are used interchangeably and refer to a site within a gene at which there is sequence variability between different individuals in the population and/or between different alleles of the gene. Where a polymorphic site is a single nucleotide in length, the site is referred to as a single nucleotide polymorphism (SNP). Polymorphic sites may be several nucleotides in length due to insertions, deletions, conversions or translocations. The National Center for Biotechnological Information (NCBI) has assigned an official identification tag for each unique SNP, termed RefSNP ID (rs ID) and marked by the letters “rs” followed by a reference number. Each rs ID has been linked to specific variable alleles present in a specific nucleotide position in the human genome, and contains marking of the SNP within its flanking nucleotide sequences.

Each version of a gene sequence with respect to a polymorphic site is referred to herein as an “allele” or “variant” of the polymorphic site. According to some embodiments, an allele refers to a gene sequence comprising at least one polymorphism in at least one site. According to some embodiments, an allele of a gene may comprise several SNPs. According to some embodiments, the terms “allele” or “variant” may refer to versions of a peptide encoded by a gene comprising a change in at least one polymorphic site.

The terms “patient”, “subject” and “individual”, as used herein, are used interchangeably and refer to a mammalian (e.g., human) subject to be treated and/or from which a biological sample is derived. According to some embodiments, a subject is a human subject. According to some embodiments, the subject is afflicted with a condition which may benefit from administration of bisphosphonate. According to some embodiments, a healthy subject, as used herein, is a subject who is not predisposed for BONJ following bisphosphonate administration, a subject free from BONJ symptoms or a combination thereof. Each possibility represents a separate embodiment of the present invention.

The terms “anti-resorptive-agent-induced osteonecrosis of the jaw” and “ARONJ” as used herein are interchangeable with any one of the terms “bisphosphonate-induced osteonecrosis of the jaw”, “BONJ”, “BRONJ” and/or the terms osteonecrosis of the jaw and “ONJ”.

As used herein, the expressions “a subject predisposed for ARONJ”, “a subject predisposed to develop ARONJ” and “a subject predisposed for ARONJ following bisphosphonate administration” are used interchangeably and refer to a subject having genetic makeup which increases the chance of the subject to show symptoms of ARONJ following treatment with an anti-resorptive agent.

A genetic makeup which increases the chance of a subject to show symptoms of ARONJ following treatment with an anti-resorptive agent comprises having at least one single nucleotide polymorphism within the Mucin-4 gene, typically within Exon 2 of the MUC4 gene, or fragments thereof. Each possibility represents a separate embodiment of the present invention. As exemplified herein below, SNPs which are significantly associated with susceptibility to ARONJ are found within the MUC4 gene or Exon 2 of the MUC4 gene, preferably within Ser, Thr or Pro residues associated with O-glycosilation of the alpha subunit of MUC4 encoded by Exon 2.

According to some embodiments, a sample obtained from a subject is a sample comprising polynucleotide, DNA and/or RNA. According to some embodiments, the sample comprises at least one mRNA molecule encoded by the Mucin-4 gene.

According to some embodiments, the sample obtained from a subject is a sample which is processed by one or more of: (a) fragmentation of the polynucleotides within the sample; or (b) purification of the polynucleotides and/or fragments thereof from the sample, or a combination thereof. Each possibility represents a separate embodiment of the present invention.

According to some embodiments, fragmentation of polynucleotides is performed by a method selected from: at least one restriction enzyme, acoustic shearing (such as by sonication), pressure shearing (such as by French press apparatus), at least one chemical reagent or a combination thereof. Each possibility represents a separate embodiment of the present invention.

According to some embodiments, purification of the polynucleotides and/or fragments thereof from the sample refers to purifying the sample such that it contains less than 20%, preferably less than 10%, most preferably less than 5% cell constituents other than polynucleotides. Each possibility represents a separate embodiment of the present invention.

According to some non-limiting embodiments, identifying the presence of at least one single nucleotide polymorphism in the sample derived from said is facilitated using a method selected from the group consisting of: sequencing, next generation sequencing (also known as exome sequencing), Restriction Fragment Length Polymorphism (RFLP), DNA Microarray, SNP Microarray, Polymerase Chain Reaction (PCR), Reverse-Transcription Polymerase Chain Reaction (RT-PCR), denaturing HPLC and gel electrophoresis, Single Strand Conformation Polymorphism and gel electrophoresis, DNA hybridization analysis Mass Spectrometry and a combination thereof. Each possibility represents a separate embodiment of the present invention.

As used herein, the term “exome” refers to the part of the genome formed by exons. The exome differs from a transcriptome in that it consists of all DNA that is transcribed into mature RNA in cells of any type.

According to some embodiments, determining the presence of at least one single nucleotide polymorphism within a sample comprises:
(a) reacting at least one biological probe with at least one biomarker within the sample, said biological probe being configured to specifically hybridize to said at least one biomarker indicative of a subject's predisposition to ARONJ; and
(b) using at least one detection reagent to detect hybridization between said at least one biomarker and at least one biological probe, said hybridization being indicative of the presence of said at least one single nucleotide polymorphism within said sample.

According to some embodiments, determining the presence of at least one single nucleotide polymorphism within a sample according to the present invention comprises:
(a) reacting a plurality of biological probes with a plurality of biomarkers within the sample, said biological probes being configured to specifically hybridize to at least one biomarker indicative of a subject's predisposition to ARONJ; and
(b) using at least one detection reagent to detect hybridization between at least one biomarker and at least one biological probe, said hybridization being indicative of the presence of said at least one single nucleotide polymorphism within said sample.

As used herein, the terms “analyzing”, “identifying” and “determining” are interchangeable and refer to identification of at least one SNP in a sample

The term “biological probe” as used herein refers to any one or more of a cDNA probe, mRNA probe, RNA probe or an antibody, configured to identify at least one SNP in MUC4 gene, gene product or fragments thereof.

The term “biomarker” as used herein refers to a SNP for predisposition to ARONJ, including any one of the SNPs listed in Tables 2, 3 and 5, or gene, gene products or gene fragments including same, such as, the polynucleotide sequences set forth in SEQ ID NO: 1-47 and 51-55.

The at least one detection reagent may be any detection reagent known in the art, such as, but not limited to a fluorescent marker, horseradish peroxidase, a substrate cleavable by an enzyme to produce a color reaction and the like. According to some embodiments, the hybridization between matching biomarker and biological probe is strong enough to withstand at least one washing step.

According to some embodiments, the identifying the presence of at least one single nucleotide polymorphism comprises applying next generation sequencing.

Next-generation sequencing is the preferred technology for genome sequencing which is required in order to identify the SNPs of the invention in a biological sample for determining predisposition to ARONJ. Next-generation sequencing refers to non-Sanger-based high-throughput DNA sequencing technologies Millions of DNA strands can be sequenced in parallel, yielding substantially more throughput and minimizing the need for the fragment-cloning methods that are often used in Sanger sequencing of genomes. Template preparation consists of building a library of nucleic acids and amplifying that library. Sequencing libraries are constructed by fragmenting the sample and ligating adapter sequences (synthetic oligonucleotides of a known sequence) onto the ends of the DNA fragments. Once constructed, libraries are clonally amplified and sequenced. Once sequencing is complete, raw sequence data must undergo several analysis steps, including preprocessing the data to remove adapter sequences and low-quality reads, mapping of the data to a reference genome and analysis of the compiled sequence.

According to some embodiments, the fragment of MUC4 is exon 2 as set forth in SEQ ID NO: 49.

According to some embodiments, the fragment of MUC4 is a fragment of Exon 2 in MUC4, as set forth in SEQ ID NO: 50, SEQ ID NO: 56 and SEQ ID NO: 57. Each possibility represents a separate embodiment of the present invention.

As used herein, the term “exon” refers to a nucleotide sequence within a gene corresponding to a sequence which remains present within the final mature RNA product of that gene after introns have been removed by RNA splicing. According to some embodiments, the term exon refers to both the DNA sequence within a gene and to the corresponding sequence in RNA transcripts. In RNA splicing, introns are removed and exons are covalently joined to one another as part of generating the mature messenger RNA or noncoding RNA product of a gene. According to some embodiments, a polymorphism within an exon results in detectable variations in the peptide encoded by the gene comprising the exon. According to some embodiments, detectable variations in a peptide encoded by a gene comprising a certain exon are variation from the wild-type form of the peptide. According to some embodiments, detectable variations in a peptide encoded by a gene comprising a certain exon may be detected by analyzing immune-reactivity, such as, but not limited to, the ability of the peptide to bind at least one antibody having specificity towards said peptide.

According to some embodiments, said at least one single nucleotide polymorphism is selected from SNP Nos: 101-147 and 151-156. Each possibility represents a separate embodiment of the present invention.

According to some embodiments, the method further comprises obtaining a sample from said subject prior to said identifying.

According to some embodiments, the sample is a sample of bodily fluids. According to some embodiments, the sample is selected from the group consisting of: blood, serum, plasma, urine, sweat, buccal smear, saliva or a combination thereof. Each possibility represents a separate embodiment of the present invention. According to some embodiments, the sample is a saliva sample. According to other embodiments, the sample is a blood sample.

According to some embodiments, said at least one single nucleotide polymorphism comprises a plurality of single nucleotide polymorphisms.

As used herein, the term “a plurality” refers to at least 2. According to some embodiments, the term “a plurality” refers to at least 2, 3, 4, 5, 6, 7, 8, 9, 10, 15, 20, 25, 30, 35, 40 or 42. Each possibility represents a separate embodiment of the present invention. According to some embodiments, at least one single nucleotide polymorphism refers to at least 2, 3, 4, 5, 6, 7, 8, 9, 10 single nucleotide polymorphisms. Each possibility represents a separate embodiment of the present invention.

According to some embodiments, at least one single nucleotide polymorphism refers to at least 10, 20, 30, 40 single nucleotide polymorphisms. Each possibility represents a separate embodiment of the present invention.

According to some embodiments, at least one single nucleotide polymorphism refers to at least 2, 3, 4, 5, 6, 7, 8, 9, 10, 15, 20, 25, 30, 35, 40, 42 single nucleotide polymorphisms as set forth in Tables 2, 3 and 5. According to some embodiments, at least one single nucleotide polymorphism refers to at least 2, 3, 4, 5, 6, 7, 8, 9, 10, 15, 20, 25, 30, 35, 40, 42, 45, 47 single nucleotide polymorphisms as set forth in Tables, 3 and 5. Each possibility represents a separate embodiment of the present invention.

According to some embodiments, the at least one single nucleotide polymorphism refers to a plurality of single nucleotide polymorphisms within a plurality of haplotypes. According to some embodiments, the at least one single nucleotide polymorphism refers to a plurality of single nucleotide polymorphisms within a single haplotype.

According to some embodiments, said at least one single nucleotide polymorphism comprises at least 5 single nucleotide polymorphisms.

According to some embodiments, said at least one single nucleotide polymorphism comprises at least 10 single nucleotide polymorphisms.

Any combination of alleles which are usually inherited together is termed “haplotype”. According to some embodiments, a haplotype refers to any combination of SNPs which are usually inherited together. According to some embodiments, a haplotype refers to a combination of at least two alleles. According to some embodiments, a haplotype refers to a combination of at least two SNPs.

According to some embodiments, said gene product is an mRNA molecule.

According to some embodiments, the gene product is mRNA corresponding to transcript 1 of the MUC4 gene, as set forth in SEQ ID NO: 49, or part thereof.

According to some embodiments, analyzing an mRNA molecule for the presence of at least one SNP comprises hybridizing said mRNA molecule with a polynucleotide probe configured to specifically bind mRNA comprising said at least one SNP.

It is to be understood that an mRNA molecule comprising a single nucleotide polymorphism may refer to an mRNA molecule encoded by DNA comprising said single nucleotide polymorphism.

According to some embodiments, analyzing an mRNA molecule for the presence of at least one SNP comprises hybridizing a cDNA corresponding to said mRNA with a polynucleotide probe configured to specifically bind a cDNA comprising said at least one SNP.

According to some embodiments, the polynucleotide probe is a DNA probe, an RNA probe or a combination thereof. Each possibility represents a separate embodiment of the present invention.

As used herein, the terms “DNA probe” and “RNA probe” refer to a single strand of DNA or RNA polynucleotides, respectively, which are able to specifically bind a target polynucleotide or fragments thereof.

According to some embodiments, the target polynucleotide is DNA encoding the Mucin-4 gene or part thereof having at least one SNP within the region encoding Exon2 of Mucin-4 mRNA transcript 1, said exon spanning positions 195505661-195518368 of chromosome 3, as set forth in SEQ ID NO: 49. According to some embodiments, the target polynucleotide is DNA encoding the Mucin-4 gene or part thereof having at least one SNP within positions 195505883-195515290 of chromosome 3, as set forth in SEQ ID NO: 50. According to some embodiments, the target polynucleotide is DNA encoding the Mucin-4 gene or part thereof having at least one SNP within positions 195505884-195508789 of chromosome 3, as set forth in SEQ ID NO: 56. According to some embodiments, the target polynucleotide is DNA encoding the Mucin-4 gene or part thereof having at least one SNP within positions 195513831-195515290 of chromosome 3, as set forth in SEQ ID NO: 57.

According to some embodiments, the target polynucleotide is mRNA encoded by the Mucin-4 gene or part thereof having at least one SNP within the region encoding Exon2 of Mucin-4 mRNA transcript 1, said exon spanning positions 195505661-195518368 of chromosome 3, as set forth in SEQ ID NO: 49. According to some embodiments, the target polynucleotide is mRNA encoded by the Mucin-4 gene or part thereof having at least one SNP within positions 195505883-195515290 of chromosome 3, as set forth in SEQ ID NO: 50 or within positions 195505884-195508789 of chromosome 3, as set forth in SEQ ID NO: 56, or within positions 195513831-195515290 of chromosome 3, as set forth in SEQ ID NO: 57.

According to some embodiments, said polynucleotide probe is configured to specifically bind mRNA comprising at least one SNP as set forth in Tables 2, 3 and 5 (SNP NO: 101-147 and 151-156). Each possibility represents a separate embodiment of the present invention.

According to some embodiments, the polynucleotide probe is selected from SEQ ID NOs: 1-47 and 21-55. Each possibility represents a separate embodiment of the present invention.

According to some embodiments, the polynucleotide probe is configured to specifically hybridize the Mucin-4 gene or a fragment thereof comprising at least one SNP as set forth in Tables 2, 3 and 5. According to other embodiments, the polynucleotide probe is configured to specifically hybridize an mRNA encoded by the Mucin-4 gene comprising at least one SNP as set forth in Tables 2, 3 and 5. According to some embodiments, the polynucleotide probe is configured not to bind the wild-type Mucin-4 gene or an mRNA encoded by it. According to some embodiments, the polynucleotide probe is configured to bind the wild-type Mucin-4 gene or an mRNA encoded by it to a significantly lower extent than the binding of the Mucin-4 gene or mRNA comprising at least one SNP as set forth in Tables 2, 3 and 5.

According to some embodiments, the polynucleotide probe has a length of at least 18 bases, optionally at least 20 bases, typically at least 25 bases. Each possibility represents a separate embodiment of the present invention.

As used herein, a “cDNA comprising a single nucleotide polymorphism” refers to a cDNA encoded by an mRNA which in turn is encoded by a genomic sequence comprising said single nucleotide polymorphism.

According to some embodiments, said identifying, comprises using a polynucleotide probe for specific hybridization to said mRNA molecule.

According to some embodiments, said gene product is a protein. According to some embodiments, said identifying comprises applying an antibody capable of binding to said protein.

As used herein, the terms “protein” and “polypeptide” are used interchangeably and refer to a sequence comprising a plurality of amino acids. According to some embodiments, the term “peptide” refers to a sequence comprising a plurality of amino acids, possibly about 5-20 amino acids, alternatively about 10-20 amino acids. Each possibility represents a separate embodiment of the present invention.

According to some embodiments, the gene product is a protein, or a part thereof, encoded by the MUC 4 gene or encoded by transcript 1 of the MUC 4 gene, as set forth in SEQ ID NO: 49. Each possibility represents a separate embodiment of the present invention.

According to some embodiments, analyzing a protein, or a part thereof, for the presence of at least one SNP, comprises binding said protein or fragment thereof with an antibody configured to specifically bind a protein or protein fragment encoded by a gene comprising said at least one SNP.

It is to be understood that a protein or fragment thereof comprising a single nucleotide polymorphism may refer to a protein or fragment thereof encoded by DNA comprising said single nucleotide polymorphism.

According to some embodiments, said subject is a subject having a disease or disorder requiring anti-resorptive therapy.

According to some embodiments, said subject is a subject having a condition which may benefit from administration of bisphosphonate.

According to some embodiments, said disease or disorder is selected from the group consisting of: osteoporosis, osteitis deformans, osteogenesis imperfecta, bone metastasis, breast cancer, prostate cancer, lung cancer and multiple myeloma. Each possibility represents a separate embodiment of the present invention.

According to some embodiments, a condition which may benefit from administration of bisphosphonate is a condition affecting bone metabolism. According to some embodiments, a condition which may benefit from administration of bisphosphonate is selected from the group consisting of: osteoporosis, osteitis deformans (Paget's disease of the bone), osteogenesis imperfecta, bone metastasis (with or without hypercalcaemia) and multiple myeloma. Each possibility represents a separate embodiment of the present invention.

According to some embodiments, a condition which may benefit from administration of bisphosphonate is a condition which may benefit from systemic administration of bisphosphonate. According to some embodiments, systemic administration is intravenous (IV) administration. According to some embodiments, a condition which may benefit from administration of bisphosphonate is a malignancy having skeletal metastases, such as, but not limited to, breast cancer. As used herein, the phrases “a disease or disorder requiring bisphosphonate therapy” and “a condition which may benefit from administration of bisphosphonate” are used interchangeably.

According to some embodiments, the method further comprising administering to said subject a composition comprising at least one anti-resorptive agent not associated with ARONJ or having a low probability of inducing ARONJ.

As used herein, the terms “bone anti-resorptive agent” and “anti-resorptive agent” may be used interchangeably and refer to agents which decrease or prevent bone resorption processes by means such as, but not limited to, inhibiting the activities and functions of osteoclasts (bone resorbing cells) and/or perturbing the differentiation of osteoblasts (bone forming cells). According to some embodiments, bone anti-resorptive agents are bisphosphonates. According to some embodiments, bisphosphonate-induced osteonecrosis of the jaw (BONJ or BRONJ) is a type of anti-resorptive-agent-induced osteonecrosis of the jaw (ARONJ). According to some embodiments, anti-resorptive therapy refers to therapy comprising administration of at least one bone anti-resorptive agent. According to some embodiments, anti-resorptive therapy is bisphosphonate therapy. According to some embodiments, a disease or disorder requiring bisphosphonate therapy is a disease or disorder requiring anti-resorptive therapy.

According to some embodiments, said at least one anti-resorptive agent is selected from the group consisting of: bisphosphonate not associated with ARONJ, bisphosphonate having a low probability of inducing ARONJ, bisphosphonate at a low dosage, a non-bisphosphonate anti-resorptive agent having a low probability of inducing ARONJ, a non-bisphosphonate anti-resorptive agent not associated with ARONJ and a combination thereof.

According to some embodiments, said bisphosphonate having a low probability of inducing ARONJ is selected from the group consisting of: clondronate, etidronate, tiludronate, risedronate, ibandronate and a combination thereof.

As used herein, the term “bisphosphonates” relates to drugs having two phosphonate (PO₃) groups attached to a central (geminal) carbon to which two further side chains, R₁ and R₂, are attached. Bisphosphonates are commonly divided into nitrogenous bisphosphonates which contain nitrogen and non-nitrogenous bisphosphonates which do not. Nitrogenous bisphosphonates include, but are not limited to, etidronate, clodronate and tiludronate. Non-nitrogenous bisphosphonates include, but are not limited to, pamidronate, neridronate, olpadronate, alendronate, ibandronate, risedronate and zoledronic acid.

According to some embodiments, the bone anti-resorptive agent not associated with ARONJ or having a low probability of inducing ARONJ is selected from the group consisting of: bisphosphonate not associated with BONJ, bisphosphonate having a low probability of inducing BONJ, bisphosphonate at a low dosage, a non-bisphosphonate anti-resorptive agent having a low probability of inducing ARONJ, a non-bisphosphonate anti-resorptive agent not associated with ARONJ and a combination thereof. Each possibility represents a separate embodiment of the present invention.

According to some embodiments, administering a composition comprising an anti-resorptive agent comprises administering the composition at a low frequency.

According to some embodiments, there is provided a method of treating a disease or disorder requiring anti-resorptive therapy in a subject in need thereof, comprising:
identifying, in a sample derived from the subject, the presence of at least one single nucleotide polymorphism in the Mucin-4 gene, gene product or a fragment thereof; and
administering to said subject a composition comprising at least one anti-resorptive agent not associated with ARONJ or having a low probability of inducing ARONJ.

According to some embodiments, the method of treatment prevents the onset or development of, reduces the probability of, inhibits the development or progression of, suppresses and/or ameliorates manifestation of ARONJ in a subject predisposed for ARONJ following administration of an anti-resorptive agent. Each possibility represents a separate embodiment of the present invention.

According to some embodiments, preventing refers to reducing the probability of, reducing severity of or a combination thereof. Each possibility represents a separate embodiment of the present invention.

According to some embodiments, the method of treatment comprises administering to the subject a treatment for a condition which may benefit from administration of an anti-resorptive agent, such as, a bisphosphonate.

According to some embodiments, the method of treatment comprises administration of a treatment which would not induce ARONJ following administration of an anti-resorptive agent to a subject predisposed for ARONJ. According to some embodiments, the method of treatment comprises administering to of a treatment which has a low probability of inducing ARONJ following administration of an anti-resorptive agent to a subject predisposed for BONJ.

According to some embodiments, a treatment which would not induce ARONJ or has a low probability of inducing ARONJ following administration of an anti-resorptive agent to a subject predisposed for BONJ comprises administration of a composition comprising an anti-resorptive agent selected from the group consisting of: bisphosphonate not associated with bisphosphonate-induced jaw osteonecrosis (BONJ), bisphosphonate having a low probability of inducing BONJ, bisphosphonate at a low dosage, a non-bisphosphonate anti-resorptive agent having a low probability of inducing ARONJ, a non-bisphosphonate anti-resorptive agent not associated with ARONJ and a combination thereof. Each possibility represents a separate embodiment of the present invention.

According to some embodiments, a treatment which would not induce ARONJ or has a low probability of inducing ARONJ following administration of an anti-resorptive agent to a subject predisposed for ARONJ comprises use of bisphosphonate not associated with bisphosphonate-induced jaw osteonecrosis (BONJ), bisphosphonate having a low probability of inducing BONJ, bisphosphonate at a low dosage, bisphosphonate at a low administration frequency, non-bisphosphonate anti-resorptive agent having a low probability of inducing ARONJ, non-bisphosphonate anti-resorptive agent not associated with ARONJ or a combination thereof. Each possibility represents a separate embodiment of the present invention.

According to some embodiments, bisphosphonate having a low probability of inducing BONJ is selected from the group consisting of: clondronate (such as Bonefos®), etidronate (such as Didronel®), tiludronate (such as Skelid®), risedronate (such as, Actonel®), ibandronate (such as, Boniva®) and a combination thereof. Each possibility represents a separate embodiment of the present invention.

According to some embodiments, a bisphosphonate having a low probability of inducing BONJ is an orally administered bisphosphonate. According to some embodiments, orally administered bisphosphonates are selected from the group consisting of: risedronate (such as, Actonel®), ibandronate (such as, Boniva®) and a combination thereof. Each possibility represents a separate embodiment of the present invention.

According to some embodiments, administering an anti-resorptive agent at a low dosage comprises administration of an anti-resorptive agent at a dosage that is at least 10% lower than the typical dose, at least 30% lower than the typical does, or at least 50% lower than the common dose. Each possibility represents a separate embodiment of the present invention. According to some embodiments, a typical dose or common dose refers to a dose which is commonly administered, as known in the art, to a subject having a disease or disorder selected from the group consisting of: osteoporosis, osteitis deformans, osteogenesis imperfect, bone metastasis, breast cancer, prostate cancer, lung cancer and multiple myeloma. Each possibility represents a separate embodiment of the present invention.

According to some embodiments, administering an anti-resorptive agent at a low administration frequency comprises administration of an anti-resorptive agent at a frequency of less than 12 times a year, typically less than 4 times a year. Each possibility represents a separate embodiment of the present invention.

According to some embodiments, administering an anti-resorptive agent at a low administration frequency comprises administration of intra-venous anti-resorptive agent at a frequency of less than 12 times a year, typically less than 4 times a year. Each possibility represents a separate embodiment of the present invention. According to some embodiments, intra-venous administered anti-resorptive agents are bisphosphonates selected from zoledronate, pamidronate and a combination thereof. Each possibility represents a separate embodiment of the present invention.

According to some embodiments, a treatment which would not induce ARONJ following administration of an anti-resorptive agent to a subject predisposed for ARONJ, or which has a low probability of inducing ARONJ, is administration of an anti-resorptive agent for short time durations. According to some embodiments, short time duration is less than 12 months, typically less than 9 months, possibly less than 6 months. Each possibility represents a separate embodiment of the present invention. According to some embodiments, the short time durations are separated by intermissions in which bisphosphonate is not administered to the patient, or in which a different type of bisphosphonate is administered to the patient. Each possibility represents a separate embodiment of the present invention.

According to some embodiments, there is provided a kit for determining predisposition to ARONJ, the kit comprising:
at least one biological probe configured to identify one or more single nucleotide polymorphism in the Mucin-4 gene, gene product or a fragment thereof in a biological sample; and
written instructions for use of said kit for determining predisposition to ARONJ.

According to some embodiments, said biological probe comprises PCR primers configured to amplify a fragment of the MUC4 gene comprising said at one or more single nucleotide polymorphism.

According to some embodiments, said fragment is selected from SEQ ID NOs: 1-47 and 51-55.

According to some embodiments, the kit further comprises a reference probe configured to identify a fragment of the wild type MUC4 gene.

According to some embodiments, the kit further comprises at least one detection reagent.

According to some embodiments, said at least one biological probe is an antibody configured to specifically bind a MUC4 gene product comprising said at one or more single nucleotide polymorphism.

According to some embodiments, there is provided a kit for treating a disease or disorder requiring anti-resorptive therapy in a subject in need thereof, comprising:
at least one biological probe configured to identify one or more single nucleotide polymorphism in the Mucin-4 gene, gene product or a fragment thereof in a biological sample;
written instructions for use of said kit for determining predisposition to ARONJ; and
a composition comprising at least one anti-resorptive agent not associated with ARONJ or having a low probability of inducing ARONJ for treatment said disease or disorder in a subject identified as having predisposition to ARONJ.

According to some embodiments, the kit further comprises a solid support. According to some embodiments, the at least one biological probe is tethered to a solid support. According to some embodiments, the at least one biological probe is a plurality of biological probes.

According to some embodiments, a plurality of biological probes may be in the form of an array, such as, but not limited to, a polynucleotide array or a peptide array. Each possibility represents a separate embodiment of the present invention. According to some embodiments, the solid support may be made of any appropriate solid material known in the art, preferably treated such that it may promote adhesion of the biological probes. According to some embodiments, the solid support is a support used for polynucleotide or peptide microarray.

According to some embodiments, there is provided use of a composition comprising at least one anti-resorptive agent not associated with ARONJ or having a low probability of inducing ARONJ for treatment of a disease or disorder requiring anti-resorptive therapy in a subject identified as having predisposition to ARONJ.

According to some embodiments, there is provided a composition comprising at least one anti-resorptive agent not associated with ARONJ or having a low probability of inducing ARONJ for treatment of a disease or disorder requiring anti-resorptive therapy in a subject identified as having predisposition to ARONJ.

The terms “comprises”, “comprising”, “includes”, “including”, “having” and their conjugates mean “including but not limited to”. The terms “comprises” and “comprising” are limited in some embodiments to “consists” and “consisting”, respectively. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure. In the description and claims of the application, each of the words “comprise” “include” and “have”, and forms thereof, are not necessarily limited to members in a list with which the words may be associated.

As used herein, the singular form “a”, “an” and “the” include plural references unless the context clearly dictates otherwise. For example, the term “a compound” or “at least one compound” may include a plurality of compounds, including mixtures thereof.

As used herein the term “about” refers to plus/minus 10% of the value stated.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

### Example 1

### Identification of Single Nucleotide Polymorphisms in Subjects Afflicted with BOND Through Exome Sequencing—Israel Clinical Study

In order to determine whether there are SNPs within the exome of BONJ patients which differentiate them from healthy subjects, DNA was extracted from blood of 43 multiple myeloma patients and subjected to exome sequencing. The study was conducted in Israel, with a population of Caucasians 44 to 81 years old, 25 men and 18 women. All 43 patients received IV administration of bisphosphonates, primarily pamidronate, zoledronic acid (Z) or combined therapy (PZ for patients that initially received pamidronate and later zoledronic acid and ZP for the opposite sequence). Out of the 43 examined patients, 23 had BONJ and 20 did not exhibit BONJ symptoms. Table 1 summarizes the clinical data of the patients who participated in the described experiment.

TABLE 1
Clinical data of patients who participated in the Israel study

| Bisphosphonate | BRONJ | BRONJ | Blood sample | | | Subject |
|---|---|---|---|---|---|---|
| administered | starting date | symptoms | collection date | Age | Gender | Number |
| P | Aug. 20, 2006 | Yes | Feb. 21, 2012 | 66 | Male | 001 |
| P | Nov. 23, 2010 | Yes | Feb. 21, 2012 | 68 | Male | 004 |
| P | Nov. 22, 2006 | Yes | Feb. 22, 2012 | 63 | Male | 005 |
| Z | Apr. 1, 2009 | Yes | Mar. 7, 2012 | 69 | Male | 006 |
| Z | Apr. 1, 2003 | Yes | Mar. 7, 2012 | 75 | Male | 007 |
| PZ | Sep. 1, 2006 | Yes | Mar. 20, 2012 | 66 | Female | 008 |
| P | Nov. 1, 2006 | Yes | Mar. 20, 2012 | 60 | Male | 009 |
| Z | Mar. 1, 2007 | Yes | Apr. 4, 2012 | 61 | Male | 012 |
| PZ | Jun. 1, 2002 | Yes | Apr. 4, 2012 | 59 | Male | 013 |
| P | Jul. 1, 2005 | Yes | Apr. 18, 2012 | 64 | Female | 015 |
| P | Jun. 1, 2010 | Yes | Jun. 14, 2012 | 82 | Female | 018 |
| ZP | May 1, 2007 | Yes | May 29, 2012 | 75 | Male | 021 |
| Z | Jun. 1, 2006 | Yes | May 29, 2012 | 68 | Male | 022 |
| Z | May 1, 2008 | Yes | Jun. 17, 2012 | 68 | Female | 023 |
| PZ | Sep. 1, 2005 | Yes | Jun. 8, 2012 | 64 | Male | 025 |
| P | Jan. 1, 2009 | Yes | Jun. 20, 2012 | 69 | Female | 029 |
| PZ | Jan. 31, 2012 | Yes | Aug. 28, 2012 | 70 | Male | 031 |
| P | Jun. 1, 2007 | Yes | Oct. 16, 2012 | 81 | Female | 063 |
| P | UNK | Yes | Oct. 16, 2012 | 48 | Male | 064 |
| PZ | Jun. 1, 2006 | Yes | Oct. 16, 2012 | 62 | Female | 069 |
| P | Jul. 18, 2012 | Yes | Nov. 21, 2012 | 63 | Female | 072 |
| Z | UNK | Yes | Nov. 28, 2012 | 68 | Female | 081 |
| P | Dec. 1, 2011 | Yes | Dec. 25, 2012 | 81 | Female | 089 |
| P | NA | no | Apr. 17, 2012 | 66 | Female | 014 |
| P | NA | no | Apr. 24, 2012 | 62 | Female | 016 |
| P | NA | no | May 29, 2012 | 76 | Male | 017 |
| P | NA | no | May 23, 2012 | 71 | Male | 019 |
| P | NA | no | May 28, 2012 | 65 | Male | 020 |
| PZ | NA | no | May 30, 2012 | 64 | Male | 024 |
| P | NA | no | Jun. 20, 2012 | 64 | Female | 030 |
| UNK | NA | no | Jul. 20, 2012 | 60 | Female | 034 |
| P | NA | no | Jul. 9, 2012 | 66 | Male | 036 |
| P | NA | no | Dec. 12, 2012 | 49 | Male | 042 |
| P | NA | no | Aug. 26, 2012 | 71 | Female | 044 |
| P | NA | no | Aug. 28, 2012 | 69 | Female | 047 |
| P | NA | no | Sep. 11, 2012 | 62 | Female | 056 |
| ZP | NA | no | Sep. 19, 2012 | 56 | Male | 061 |
| P | NA | no | Oct. 31, 2012 | 77 | Female | 075 |
| P | NA | no | Nov. 7, 2012 | 71 | Male | 078 |
| PZ | NA | no | Nov. 13, 2012 | 54 | Male | 079 |
| UNK | NA | no | Dec. 4, 2012 | 76 | Male | 082 |
| Z | NA | no | Dec. 4, 2012 | 66 | Male | 083 |
| P | NA | no | Dec. 4, 2012 | 44 | Male | 084 |

P = Pamidronate;
Z = Zoledronic Acid;
PZ = Patient received Pamidronate and moved to Zoledronic Acid;
ZP = Patient received Zoledronic Acid and moved to Pamidronate

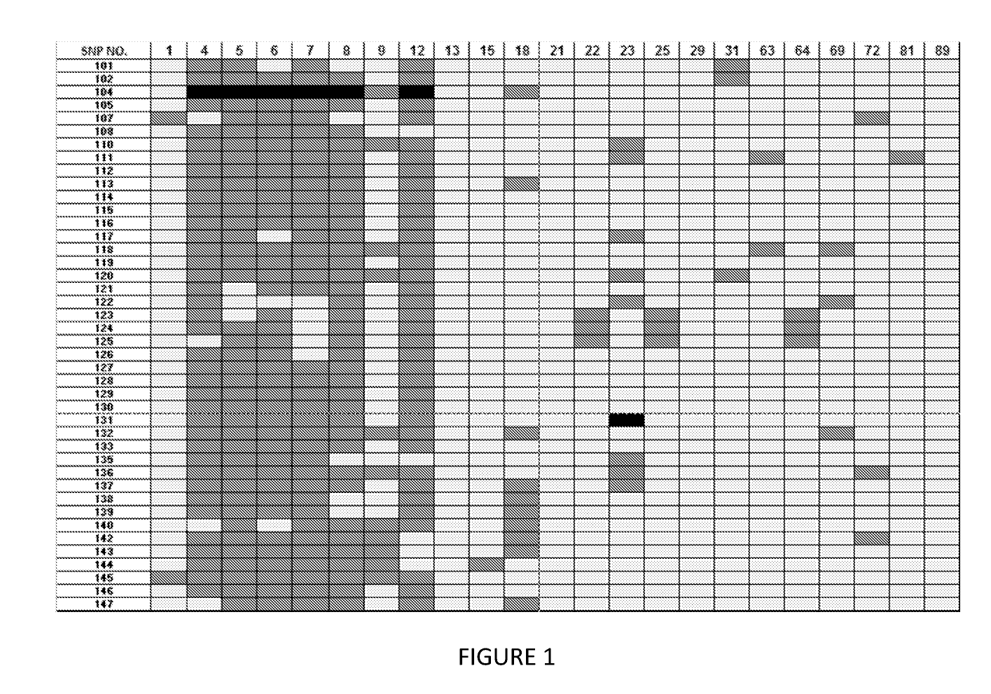

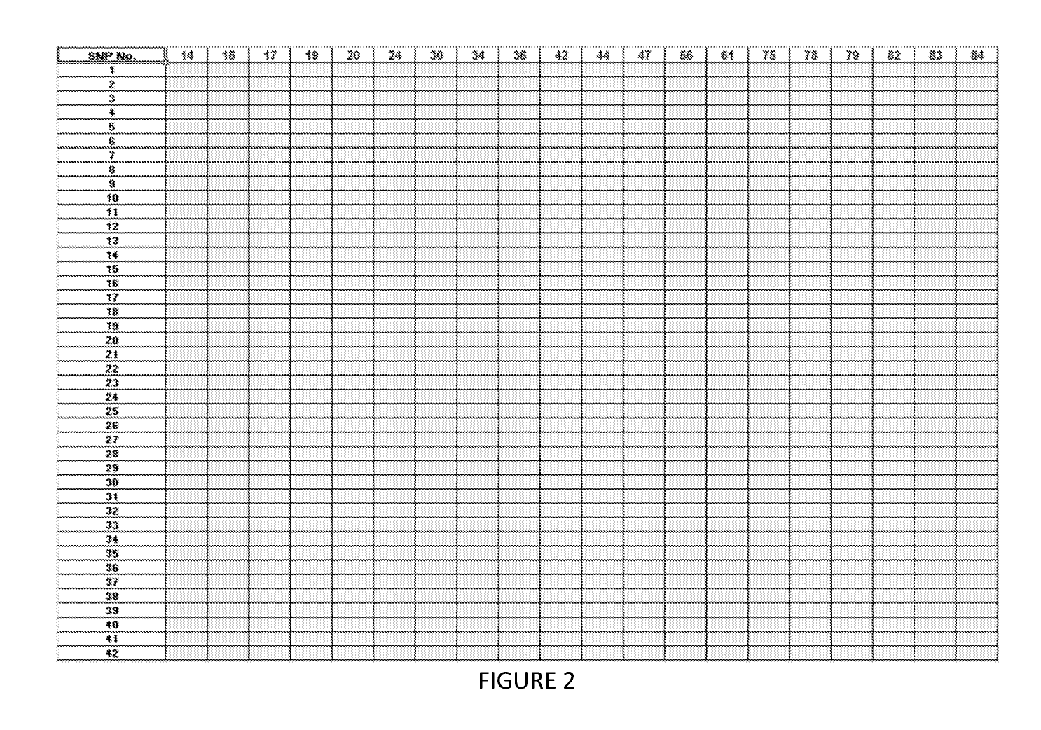

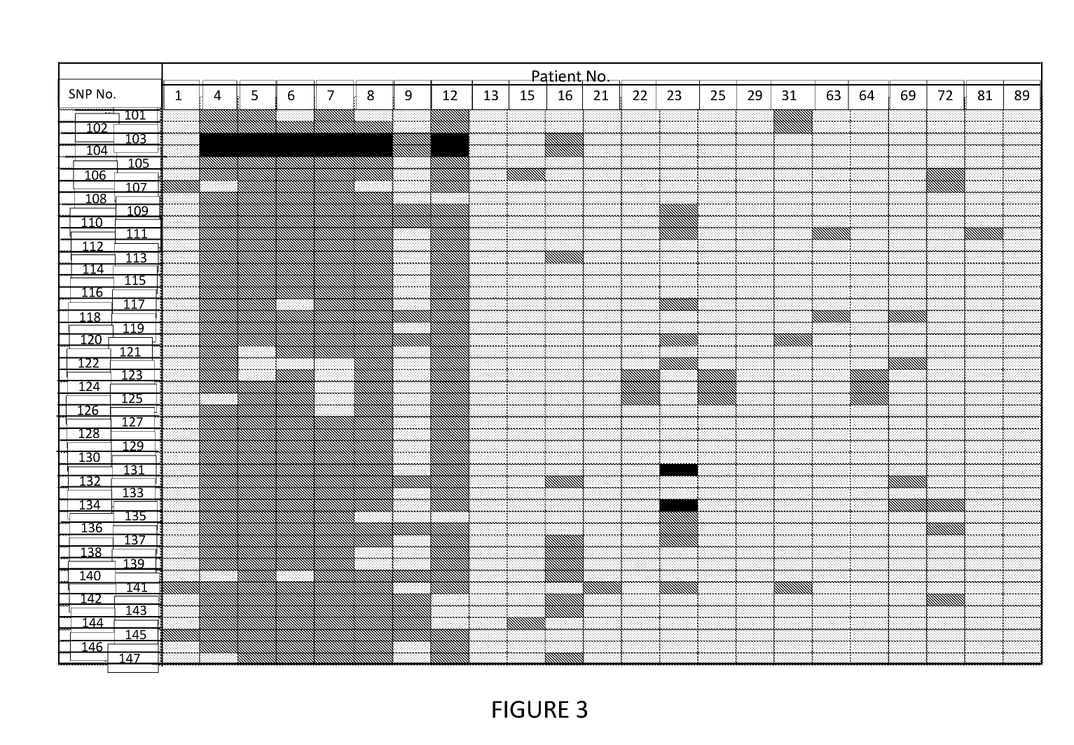

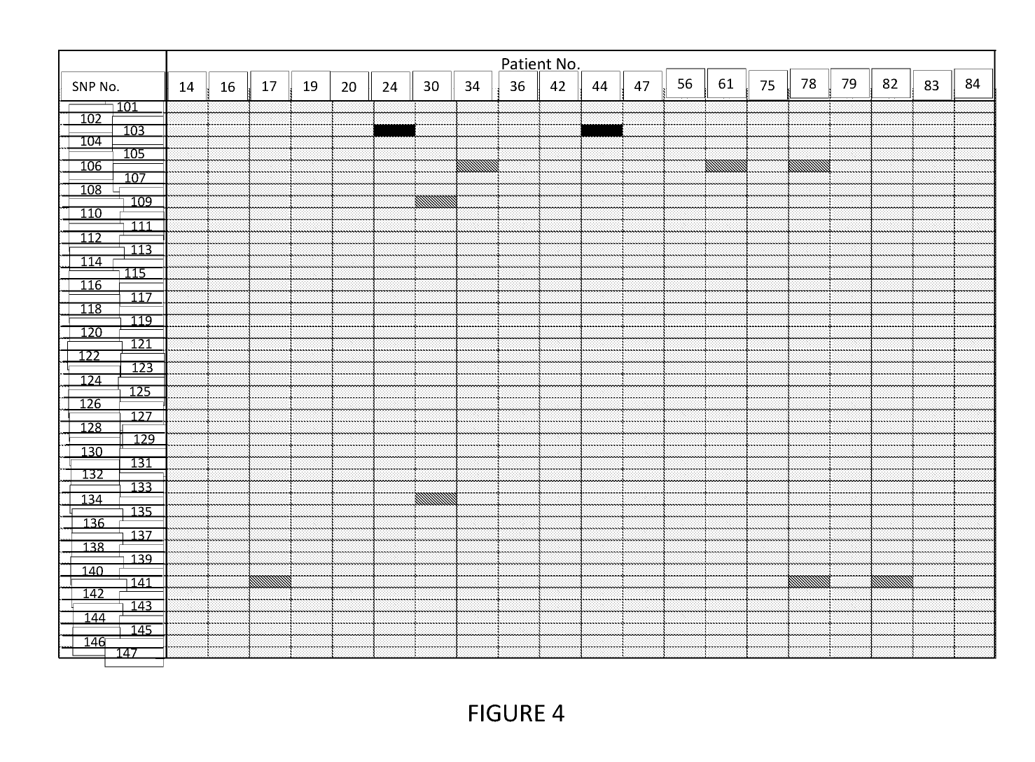

The exome sequencing analysis revealed SNPs which segregate either with BONJ patients or patients lacking BONJ symptoms. Amongst the newly discovered SNPs associated with BONJ, there are known SNPs as well as novel SNPs. Table 2 lists SNPs identified in the exome sequences which were detected in bisphosphonate-treated patients that developed BONJ but not in bisphosphonate-treated patients lacking BONJ symptoms. As indicated in the data presented in Tables 2 and 3, some of the SNPs did not result with any change in the amino acid sequences encoded by the DNA which includes the SNP (Synonymous), while other SNPs do entail change at the amino acid level (missense). Table 3 lists SNPs identified in the exome sequences which showed a significantly higher occurrence in bisphosphonate-treated patients which developed BONJ than in bisphosphonate-treated patients lacking BONJ symptoms. FIG. 1 shows the distribution of the SNPs set forth in Table 2 in bisphosphonate-treated subjects which developed BONJ, while FIG. 2 shows the distribution of the SNPs set forth in Table 2 in bisphosphonate-treated subjects lacking BONJ symptoms. FIG. 3 shows the distribution of the SNPs set forth in Tables 2 and 3 in bisphosphonate-treated subjects which developed BONJ, while FIG. 4 shows the distribution of the SNPs set forth in Tables 2 and 3 in bisphosphonate-treated subjects lacking BONJ symptoms.

TABLE 2
SNPs which were detected in bisphosphonate-treated patients that developed BONJ
but not in bisphosphonate-treated patients lacking BONJ symptoms, in the US Study

| | | | | | | | Encompassed |
|---|---|---|---|---|---|---|---|
| | Resulting | | | | | | within |
| | amino acid | Altered | Ref. | Ref SNP (RS) | Chromosomal | SNP | SEQ ID |
| P-genotype | change | allele | allele | ID | Position | No. | NO: |
| 0.057 | MISSENSE | C | T | rs200813870 | Chr3: 195505883 | 101 | 48-50, 56 |
| 0.02 | MISSENSE | G | C | n/a | Chr3: 195505886 | 102 | 48-50, 56 |
| 0.0008 | SILENT | G | C | n/a | Chr3: 195506118 | 104 | 48-50, 56 |
| 0.02 | MISSENSE | G | A | rs201273399 | Chr3: 195506137 | 105 | 48-50, 56 |
| 0.02 | MISSENSE | T | G | n/a | Chr3: 195506531 | 107 | 48-50, 56 |
| 0.057 | MISSENSE | A | G | rs200568365 | Chr3: 195506953 | 108 | 48-50, 56 |
| 0.02 | MISSENSE | T | C | rs146265282 | Chr3: 195506966 | 110 | 48-50, 56 |
| 0.005 | MISSENSE | A | G | rs142066159 | Chr3: 195506974 | 111 | 48-50, 56 |
| 0.034 | MISSENSE | A | G | rs202062831 | Chr3: 195507433 | 112 | 48-50, 56 |
| 0.02 | MISSENSE | G | T | n/a | Chr3: 195507443 | 113 | 48-50, 56 |
| 0.034 | MISSENSE | A | T | n/a | Chr3: 195507445 | 114 | 48-50, 56 |
| 0.034 | MISSENSE | T | C | n/a | Chr3: 195507446 | 115 | 48-50, 56 |
| 0.034 | MISSENSE | A | G | n/a | Chr3: 195507731 | 116 | 48-50, 56 |
| 0.034 | SILENT | T | C | rs200652820 | Chr3: 195507771 | 117 | 48-50, 56 |
| 0.008 | MISSENSE | G | C | rs201339881 | Chr3: 195507943 | 118 | 48-50, 56 |
| 0.034 | SILENT | T | A | rs200187145 | Chr3: 195507999 | 119 | 48-50, 56 |
| 0.008 | SILENT | G | A | rs79196348 | Chr3: 195508005 | 120 | 48-50, 56 |
| 0.057 | MISSENSE | T | C | rs200804425 | Chr3: 195508021 | 121 | 48-50, 56 |
| 0.057 | MISSENSE | G | C | n/a | Chr3: 195508418 | 122 | 48-50, 56 |
| 0.02 | MISSENSE | T | G | rs201933946 | Chr3: 195508451 | 123 | 48-50, 56 |
| 0.02 | MISSENSE | T | C | rs201319965 | Chr3: 195508453 | 124 | 48-50, 56 |
| 0.02 | MISSENSE | C | T | n/a | Chr3: 195508454 | 125 | 48-50, 56 |
| 0.057 | MISSENSE | A | T | rs71187746 | Chr3: 195508462 | 126 | 48-50, 56 |
| 0.057 | MISSENSE | T | C | rs80085168 | Chr3: 195508475 | 127 | 48-50, 56 |
| 0.034 | MISSENSE | C | G | rs76305071 | Chr3: 195508478 | 128 | 48-50, 56 |
| 0.034 | MISSENSE | C | T | n/a | Chr3: 195508501 | 129 | 48-50, 56 |
| 0.034 | MISSENSE | T | C | rs199896372 | Chr3: 195508502 | 130 | 48-50, 56 |
| 0.02 | MISSENSE | A | G | rs200473221 | Chr3: 195508702 | 131 | 48-50, 56 |
| 0.034 | MISSENSE | G | A | n/a | Chr3: 195508709 | 132 | 48-50, 56 |
| 0.008 | SILENT | G | A | n/a | Chr3: 195508716 | 133 | 48-50, 56 |
| 0.057 | SILENT | G | C | n/a | Chr3: 195508758 | 135 | 48-50, 56 |
| 0.02 | MISSENSE | G | A | n/a | Chr3: 195508777 | 136 | 48-50, 56 |
| 0.02 | MISSENSE | G | A | n/a | Chr3: 195508786 | 137 | 48-50, 56 |
| 0.02 | MISSENSE | T | C | n/a | Chr3: 195508789 | 138 | 48-50, 56 |
| 0.034 | MISSENSE | C | T | n/a | Chr3: 195508790 | 139 | 48-50, 56 |
| 0.034 | MISSENSE | G | T | rs75459784 | Chr3: 195511534 | 140 | 48-50 |
| 0.02 | SILENT | G | A | rs200208054 | Chr3: 195513831 | 142 | 48-50, 57 |
| 0.02 | MISSENSE | G | C | rs200981553 | Chr3: 195513846 | 143 | 48-50, 57 |
| 0.02 | MISSENSE | G | A | rs200019432 | Chr3: 195513847 | 144 | 48-50, 57 |
| 0.02 | MISSENSE | A | G | rs78359274 | Chr3: 195514450 | 145 | 48-50, 57 |
| 0.034 | MISSENSE | G | A | rs71321849 | Chr3: 195515113 | 146 | 48-50, 57 |
| 0.034 | MISSENSE | G | C | rs78535324 | Chr3: 195515290 | 147 | 48-50, 57 |

TABLE 3
SNPs which showed significantly higher occurrence in bisphosphonate-treated patients
that developed BONJ than in bisphosphonate-treated patients lacking BONJ symptoms

| | | | | | | | Encompassed |
|---|---|---|---|---|---|---|---|
| | Resulting | | | | | | within |
| | amino acid | Altered | Reference | Ref SNP (RS) | Chromosomal | SNP | SEQ ID |
| P-genotype | change | allele | allele | ID | Position | No. | NO: |
| 0.067 | SILENT | C | A | rs74941663 | Chr3: 195506091 | 103 | 48-50, 56 |
| 0.2 | MISSENSE | C | T | n/a | Chr3: 195506342 | 106 | 48-50, 56 |
| 0.05 | MISSENSE | T | C | rs199588013 | Chr3: 195506963 | 109 | 48-50, 56 |
| 0.022 | SILENT | T | G | rs201248411 | Chr3: 195508722 | 134 | 48-50, 56 |
| 0.117 | MISSENSE | A | G | rs150659095 | Chr3: 195512567 | 141 | 48-50 |

Referring to tables 2 and 3:

Chromosomal Position—chromosomal position of the SNP; Ref. (Reference allele—Nucleotide which is found at the chromosomal position in the wild-type Mucin-4 gene; Altered allele—Nucleotide which is found at the chromosomal position following the single nucleotide polymorphism; P-genotype—significance of occurrence of the SNP in bisphosphonate-treated subjects which developed BONJ as compared to bisphosphonate-treated subjects lacking BONJ symptoms according to the Fisher exact test. SNPs marked as having “n/a” RS ID are SNPs which have not been identified as of yet and thus were not assigned an RS ID.

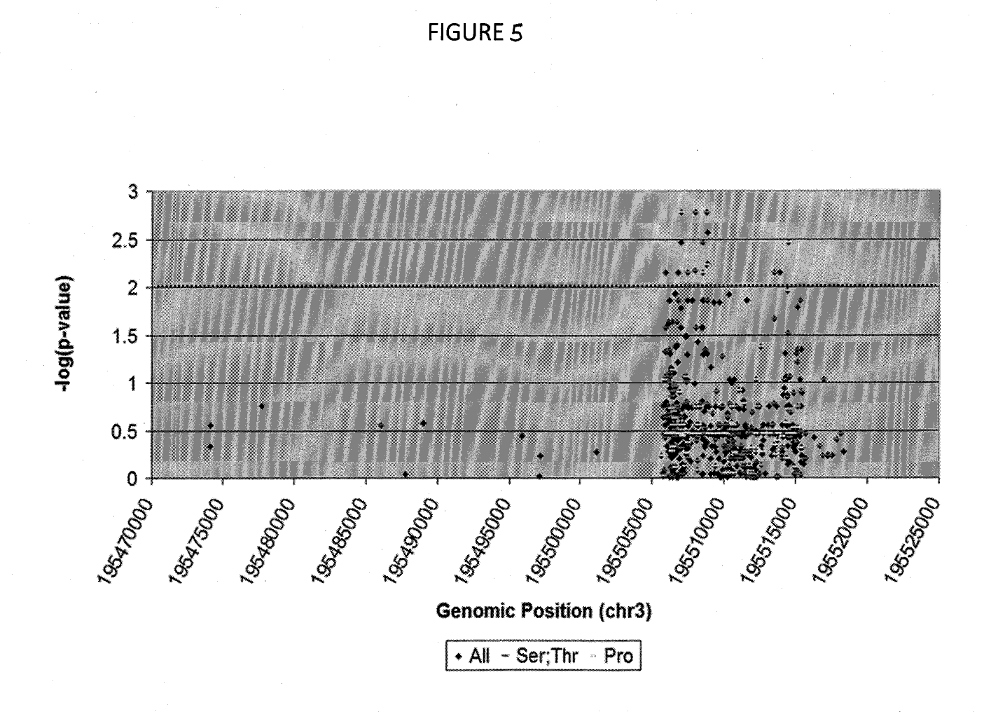

As known in the art, exon 2 of MUC4, containing a variable number of tandem repeats, is translated to the MUC4 alpha subunit that serves as a scaffold for O-glycosylation, preferably on Ser and Thr residues. As can be seen in FIG. 5, the non-synonymous SNPs along MUC4 which showed a significantly higher expression in bisphosphonate-treated subjects which developed BONJ as compared to occurrence in bisphosphonate-treated subjects lacking BONJ symptoms were concentrated within Exon 2 of MUC-4. Furthermore, a considerable amount of the SNPs associated with ONJ was found within Ser or Thr residues, which are known to serve as donor sites for O-glycosylation or within Pro residues which are preferably found in positions adjacent to the glycosylation sites. These results suggest a role for SNPs within Exon 2 of MUC4 in affecting the extent of glycosylation on MUC4.

### Example 2

### Identification of Single Nucleotide Polymorphisms in Subjects Afflicted with BONJ—U.S. Clinical Study

DNA was extracted from blood of 46 multiple myeloma patients and subjected to exome sequencing. The study was conducted in the U.S., with a mixed population of Caucasians, non-Hispanic and African Americans (AA), 33 to 81 years old, 24 men and 22 women. All 43 patients received IV administration of the following bisphosphonates: alendronate (A), pamidronate (P) and zoledronate (Z) or a combination thereof. Out of the 46 examined patients, 23 had BONJ and 23 did not exhibit BONJ symptoms. Table 4 summarizes the clinical data of the patients who participated in the described experiment.

TABLE 4
Clinical data of the patients who participated in the US study

| | | | | | | Bisphosphonate | |
|---|---|---|---|---|---|---|---|
| Sample ID | Age | Gender | Race | Ethnic | ONJ | administered | Months |
| BONJ015 | 33 | male | white | non-Hispanic | Yes | A | 87 |
| BONJ090 | 51 | female | white | non-Hispanic | Yes | A | 46 |
| BONJ010 | 52 | female | white | non-Hispanic | Yes | A | 13 |
| BONJ049 | 55 | female | white | non-Hispanic | Yes | A | 30 |
| BONJ071 | 55 | female | white | non-Hispanic | Yes | A | 55 |
| HUNL 43 | 57 | female | white | Caucasian | Yes | Z | 70 |
| BONJ006 | 57 | male | white | non-Hispanic | Yes | A | 13 |
| BONJ019 | 58 | female | white | non-Hispanic | Yes | A | 93 |
| HUNL 27 | 61 | female | white | Caucasian | Yes | Z | 36 |
| HUNL 9 | 64 | male | white | Caucasian | Yes | P, Z | 24 |
| BONJ018 | 64 | male | white | non-Hispanic | Yes | A | 11 |
| BONJ022 | 64 | male | white | non-Hispanic | Yes | A | 25 |
| BONJ020 | 66 | female | white | non-Hispanic | Yes | A | 25 |
| ONJ008 | 66 | male | white | non-Hispanic | Yes | Z | 48 |
| BONJ037 | 69 | male | AA | non-Hispanic | Yes | A | 50 |
| B0004 | 69 | male | white | non-Hispanic | Yes | Z | 11 |
| B0002 | 70 | male | white | non-Hispanic | Yes | Z/P/A | 30 |
| ONJ007 | 71 | male | white | non-Hispanic | Yes | P | 24 |
| BONJ007 | 71 | male | white | non-Hispanic | Yes | A | 24 |
| B0012 | 71 | male | white | non-Hispanic | Yes | Z | 10 |
| HUNL 7 | 79 | female | white | Caucasian | Yes | Z | 12 |
| B0011 | 79 | female | white | non-Hispanic | Yes | Z | 40 |
| HUNL 22 | 81 | female | white | Caucasian | Yes | P | 6 |
| BONJ068 | 41 | male | white | non-Hispanic | No | P | 52 |
| BONJ057 | 47 | female | white | non-Hispanic | No | Z | 25 |
| BONJ042 | 51 | female | white | non-Hispanic | No | Z | 101 |
| BONJ004 | 51 | male | white | non-Hispanic | No | Z | 27 |
| BONJ079 | 52 | female | white | non-Hispanic | No | Z | 54 |
| BONJ013 | 54 | female | white | non-Hispanic | No | Z | 27 |
| BONJ084 | 59 | female | white | non-Hispanic | No | Z | 51 |
| BONJ072 | 60 | male | white | non-Hispanic | No | Z | 20 |
| BONJ082 | 61 | male | AA | non-Hispanic | No | P | 67 |
| BONJ041 | 61 | male | white | non-Hispanic | No | P | 73 |
| BONJ027 | 62 | female | white | non-Hispanic | No | Z | 30 |
| BONJ016 | 63 | female | white | non-Hispanic | No | P | 108 |
| BONJ055 | 66 | male | white | non-Hispanic | No | Z | 50 |
| BONJ034 | 67 | male | white | non-Hispanic | No | P | 37 |
| BONJ086 | 67 | male | white | non-Hispanic | No | P | 72 |
| BONJ038 | 67 | male | white | non-Hispanic | No | Z | 25 |
| BONJ005 | 71 | female | white | non-Hispanic | No | Z | 39 |
| BONJ045 | 71 | female | white | non-Hispanic | No | Z | 75 |
| BONJ033 | 62 | male | white | non-Hispanic | No | P | 64 |
| BONJ024 | 63 | female | white | non-Hispanic | No | Z | 35 |
| BONJ060 | 72 | male | white | non-Hispanic | No | Z | 30 |
| BONJ048 | 74 | male | white | non-Hispanic | No | Z | 67 |
| BONJ017 | 74 | female | white | non-Hispanic | No | Z | 41 |

A = alendronate;
P-pamidronate
Z = zoledronate;
Months = total exposure to BPs in months;
AA African Americans.

The exome sequencing analysis revealed SNPs which segregate either with BONJ patients or patients lacking BONJ symptoms (Table 5). The newly discovered SNPs associated with BONJ, are known SNPs which were detected in bisphosphonate-treated patients that developed BONJ but were not detected in bisphosphonate-treated patients lacking BONJ symptoms. As indicated in the data presented in Table 5, one of the SNPs did not result with any change in the amino acid sequences encoded by the DNA which includes the SNP (Synonymous), while the other SNPs do entail change at the amino acid level (missense).

TABLE 5
SNPs detected in bisphosphonate-treated patients that developed BONJ but not in
bisphosphonate-treated patients lacking BONJ symptoms, in the US Study

| | | | | | | | Encompassed |
|---|---|---|---|---|---|---|---|
| | Resulting | | | | | | within |
| P- | amino acid | Altered | Ref. | Ref SNP | Chromosomal | SNP | SEQ ID |
| genotype | change | allele | allele | (RS) ID | Position | No. | NO: |
| 0.03152 | SYNONYMOUS | G | A | rs62282502 | chr3: 195515271 | 151 | 48-50, 57 |
| 0.0346 | MISSENSE | A | G | rs62284986 | chr3: 195474159 | 152 | 48 |
| 0.04252 | MISSENSE | T | C | rs71635074 | chr3: 195507925 | 153 | 48-50, 56 |
| 0.07776 | MISSENSE | A | G | rs7374593 | chr3: 195513413 | 154 | 48-50, 57 |
| 0.09353 | MISSENSE | G, A | T | rs75459784 | chr3: 195511534 | 155 | 48-50 |

All of the SNPs found in this study are in MUC4 (SEQ ID NO: 48), while most are concentrated within Exon 2 of MUC-4. These results further suggest a role for SNPs within in determining susceptibility to develop BONJ in response to treatment with bisphosphonates.

### Example 3

### Identification of Subjects Afflicted with BONJ Using DNA Hybridization

A blood, saliva, urine or other biological sample is extracted from a subject and DNA is isolated and extracted using any method known in the art. The Mucin-4 locus, or a specific region thereof (such as the region encoding exon 2 of transcript 1 of Mucin-4), is optionally amplified through a PCR reaction using specific primers, as known in the art.

The DNA (or resultant amplification product) is next reacted with a DNA probe designed to specifically hybridize with the Mucin-4 gene having at least one SNP of the SNPs listed in Table 1. The DNA probe may be designed to hybridize with any one or more of the sequences listed in Table 6. The DNA (or resultant amplification product) may alternatively be reacted with several such DNA probes, each designed to bind to the Mucin-4 gene having a different SNP or SNP combination out of the SNPs set forth in Table 1. The DNA probes are designed not to bind to the wild-type MUC4 gene, or to bind to the MUC4 gene with a significantly lower affinity.

The DNA probes are bound to a detection reagent designed to identify specific binding of the DNA probe (such as a fluorescent reagent designed to emit fluorescence upon probe binding). Different probes may comprise different fluorescent markers, such as markers of different colors, enabling detection of different SNPs in the same sample. Specific interaction between the probe and DNA obtained from the sample reveals the presence of the examined SNPs in the subject from which the sample was extracted. Such interaction is indicative of the subject's predisposition to BONJ following bisphosphonate administration.

### Example 4

### Identification of Subjects Afflicted with BOND Using ELISA or ELISA-Like Platforms

Since the SNPs of the invention are within an exon of the MUC4 gene, they may be detectable in a protein encoded by the gene. In order to identify the presence of the SNPs of the invention by analyzing a protein encoded by the MUC4 gene, a saliva or blood sample is taken from a subject and a whole protein extract is prepared from the sample using any method known in the art. The proteins derived from the sample are then reacted with an antibody which is designed to recognize a Mucin-4 protein (or a fraction of the protein) which is coded by the MUC-4 gene having at least one single nucleotide polymorphism as set forth in Table 1. Analysis is carried out through enzyme-linked immunosorbent assay (ELISA) or the like, which enable to identify the protein of interest using antibodies and a detectable change (e.g. color). The antibody is unable to recognize the wild-type Mucin-4 protein or is able to recognize the wild-type protein to a significantly lower extent than the protein encoded by the MUC-4 gene having said at least one SNP.

The antibody used may be designed to recognize the Mucin-4 protein coded by the MUC4 gene with one or more of said SNPs. Instead of a single antibody, a mixture of antibodies may be used, each designed to specifically hybridize with the Mucin-4 protein encoded by the MUC4 gene having a different combination of mutations.

The antibodies used are chemically bound to a detection reagent such as an enzyme (e.g., horseradish peroxidase) or a color reagent (e.g., fluorescent marker). The detection reagent enables detection of a specific interaction between the antibody and a protein derived from the sample. A positive interaction shows that the MUC4 gene in the subject comprises the SNPs examined by the antibody, indicating the subject's predisposition to BONJ following bisphosphonate administration. When a mixture of antibodies is used, the intensity of the reaction of the detection reagent may be correlated to the number of mutations within the MUC4 gene.

TABLE 6
sequences enclosing the SNPs as disclosed herein (Tables 2,
3 and 5) flanked by the surrounding nucleotides:

| | SEQ |
|---|---|
| | ID |
| Sequence | NO: |
| GTGTCGGTGACAGGAAGAGGGGTGGCGTCACCTGTGGATGCTGAGGAAGTG | 1 |
| TCGGTGACAGGAAGAGGGGTGGTGTGACCTGTGGATGCTGAGGAAGTGCTG | 2 |
| GAGGGGTGGCCTGACCTGTGGATGCCGAGGAAGTGTCGGTGACAGGAAGAG | 3 |
| AGGAAGTGTCGGTGACAGGAAGAGGGGTGGTGTCACCTGTGGATACTGAGG | 4 |
| AAGAGGCGTGGTGTCACCTGTGGATGCTGAGGAAAGGCTGGTGAGAGGAAG | 5 |
| TCACCTGTGGATGCTGAGGAAGTGCCGGTGACAGGAACAGGGGTGGCGTGA | 6 |
| GCGTGACCTGTGGATGCTGAGGAACTGCTGGTGACAGGAAGAGAGGTGGCG | 7 |
| AAGAGGGGTGGCGTGACCTGTGGATACTGAGGAAGCGTCGGTGACAGGAAG | 8 |
| GCGTGACCTGTGGATGCTGAGGAAGTGTCGGTGACAGGAAGAGGGGTGGTG | 9 |
| TGACCTGTGGATGCTGAGGAAGCGTTGGTGACAGGAAGAGGGGTGGTGTCA | 10 |
| GGATGCTGAGGAAGCGTCGGTGACAAGAAGAGGGGTGGTGTCACCTGTGGA | 11 |
| AAGACGGGTGGTGTCACCTGTGGATACTGAGGAAGTGTCGGTGACAGGAAG | 12 |
| GTGTCACCTGTGGATGCTGAGGAAGGGTCGGTGACAGGAAGAGGGGTGGCG | 13 |
| GTCACCTGTGGATGCTGAGGAAGTGACGGTGACAGGAAGAGGGGTGGCGTG | 14 |
| TCACCTGTGGATGCTGAGGAAGTGTTGGTGACAGGAAGAGGGGTGGCGTGA | 15 |
| GTGTCACCTGTGGATACTGAGGAAAAGCTGGTGACAGGAAGAGGGGTGGCC | 16 |
| GAGGGGTGGCCTGACCTGTGGATGCTGAGGAAGCGTCGGTGACAGGAAGAG | 17 |
| GGAAGCGCCGGTGACAGGAAGAGTGGTGGTGTCACCTGTGGATGCTGAGGA | 18 |
| TGGTGACATGAAGAGGGGTGGTGTGTCCTGTAGATGCTGAGGAAGGGCTGG | 19 |
| CATGAAGAGGGGTGGTGTGACCTGTGGATGCTGAGGAAGGGCTGGTGACAG | 20 |
| GTGACCTGTAGATGCTGAGGAAGGGTTGGTGACAGGAAGAGGGGTGGTGTC | 21 |
| ACTGAGGAAGTGTCGGTGACAGGCAGAGGGGTGGTGTCACCTGTGGATGCT | 22 |
| GTGTCACCTGTGGATGCTGAGGAAGTGCTGGTGACATGAAGAGGGGTGGCG | 23 |
| GTCACCTGTGGATGCTGAGGAAGGGTTGGTGACATGAAGAGGGGTGGCGTG | 24 |
| TCACCTGTGGATGCTGAGGAAGGGCCGGTGACATGAAGAGGGGTGGCGTGA | 25 |
| GGATGCTGAGGAAGGGCTGGTGACAAGAAGAGGGGTGGCGTGACCTGTGGA | 26 |
| GGGCTGGTGACATGAAGAGGGGTGGTGTGACCTGTGGATGCTGAGGAAGCG | 27 |
| CTGGTGACATGAAGAGGGGTGGCGTCACCTGTGGATGCTGAGGAAGCGTCG | 28 |
| GTGACCTGTGGATGCTGAGGAAGCGCCGGTGACAAGAAGAGGAGTGGCGTG | 29 |
| TGACCTGTGGATGCTGAGGAAGCGTTGGTGACAAGAAGAGGAGTGGCGTGA | 30 |
| GGATGCTGAGGAAGTGTCGGTGACAAGAAGAGAGGTGGCATGACCGGTGGA | 31 |
| GAGGAAGTGTCGGTGACAGGAAGAGGGGTGGCATGACCGGTGGATGCTGAG | 32 |
| TGTCGGTGACAGGAAGAGAGGTGGCGTGACCGGTGGATGCTGAGGAAGGGC | 33 |
| TGACAGGAAGAGAGGTGGCATGACCTGTGGATGCTGAGGAAGGGCTAGTGA | 34 |
| AGGAAGGGCTAGTGACAGGAAGAGGGGTGGTGTCACCTGTGGATACTGAGG | 35 |
| AAGAGGCGTGGTGTCACCTGTGGATGCTGAGGAAAGGCTGGTGACAGGAAG | 36 |
| GGTGTCACCTGTGGATACTGAGGAAGGGCTGGTGACAGGAAGAGGGGTGGC | 37 |
| GTCACCTGTGGATACTGAGGAAAGGTTGGTGACAGGAAGAGGGGTGGCCTG | 38 |
| TCACCTGTGGATACTGAGGAAAGGCCGGTGACAGGAAGAGGGGTGGCCTGA | 39 |
| GGATGCTGAGGAAGCGTCGGTGACAGGAAGAGGGGTGGCGTGACCTGTGGA | 40 |
| GGAAGAGAGGTGGCGTGACCTGTGGATACTGAGGAAGCGTCGGTGACAGGA | 41 |
| CACCTGTGGATGCTGAGGAAGGGCTGGTGACAGGAAGAGGCATGGTGTCAC | 42 |
| AGGAAGGGCTAGTGACAGGAAGAGGGATGGTGTCACCTGTGGATGCTGAGG | 43 |
| GGAAGGGCTAGTGACAGGAAGAGGCGTGGTGTCACCTGTGGATGCTGAGGA | 44 |
| GGTGTCACCTGTGGATGCTGAGGAAAGGCTAGTGACAGGAAGAGGCATGGT | 45 |
| AAGAGGGGTGGTGTGACCTGTGGATGCTGAGGAAGTGTCGGTGACAGGAAG | 46 |
| AGTGTCAGTGACAGGAAGAGGGGTGGTGTCACCTGTGGATGCTGAGGAAAA | 47 |
| GACCTGTGGATGCTGAGGAAGTGTCAGTGACAGGAAGAGGGGTGCTGTCAC | 51 |
| CCCCAGCAGCAAGAGGCCGCCCAGGGCCCCAAAGAAGATGCCGAAGAACGC | 52 |
| GTGACCGGTGGATGCTGAGGAAGCGCCGGTGACAGGAAGAGTGCTGGTGTC | 53 |
| GAGGAAAGGCCGGTGACAGGAAGAGGGGTGGCGTGACCGGTGGATGCTGAG | 54 |
| GGATGCTGAGGAAGCGTCGGTGACATGAAGAGGGGTGGCGTGACCTGTGGA | 55 |

### Example 5

### Identification of a Subject Afflicted with BONJ Using PCR

Since the SNPs of the invention are within an exon of the MUC4 gene, they may be detectable in mRNA of the gene. In order to identify the presence of SNP(s) in mRNA of MUC4 gene, a sample is taken from a subject and mRNA is extract from the sample using any method known in the art. The mRNA is transcribed to cDNA using any method known in the art. The cDNAs derived from the sample are then reacted with primers designed to recognize the MUC-4 transcript (or a fraction of the transcript) having at least one single nucleotide polymorphism as set forth in Tables 2, 3 and 5. The primers are unable to recognize the wild-type MUC-4 transcript or are able to recognize the wild-type MUC-4 transcript to a significantly lower extent than the MUC-4 transcript having said at least one SNP.

Identification of SNPs in MUC-4 (SEQ ID NO: 48), preferably, in exon 2 of MUC-4 (SEQ ID NO: 49) or within fragments of Exon 2, such as, SEQ ID NOs: 50, 56 and 57, in a sample obtained from a subject based on mRNA transcribed to cDNA, indicates that the subject is susceptible to BONJ.

### Example 6

### Identification of Subjects Afflicted with BONJ Using Sanger Sequencing

A biological sample (e.g. blood or saliva or urine) is collected from a subject. DNA is isolated and extracted from the sample using any method known in the art. The Mucin-4 locus, or a specific region thereof (such as the region encoding exon 2 of transcript 1 of Mucin-4), is amplified through a PCR reaction using specific primers, designed to amplify one or more regions encompassing the SNP(s) of interested, particularly, any one or the SNPs listed in Tables 2, 3 and 5.

One or more PCR fragment are sequenced by the Sanger method which enables a direct identification of the SNPs of the invention.

Identification of SNPs, using Sanger sequencing, in MUC-4 (SEQ ID NO: 48), preferably, in exon 2 of MUC-4 (SEQ ID NO: 49) or within fragments of Exon 2, such as, SEQ ID NOs: 50, 56 and 57, indicates that the subject is susceptible to BONJ.

### Example 7

### Identification of Subjects Afflicted with BONJ Using Next Generation Sequencing

A biological sample is collected from a subject and DNA is extracted using any method known in the art. The DNA is optionally fragmented, purified and end-ligated to oligonucleotide adapters. The libraries are sequenced by Next generation sequencing and the reads are reassembled using a known reference genome as a scaffold. The platform for sequencing may be either a standard kit targeting the whole exome or a costume kit that includes only the Mucin-4 locus, or a specific region thereof. Alignment of the resultant sequence to the wild type human genome enables a direct detection of the SNPs of the invention.

Identification of SNPs using Next generation sequencing in MUC-4 (SEQ ID NO: 48), preferably, in exon 2 of MUC-4 (SEQ ID NO: 49) or within fragments of Exon 2, such as, SEQ ID NOs: 50, 56 and 57, indicates that the subject is susceptible to BONJ.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

1-28. (canceled)

29. A method of identifying a subject having a predisposition to ARONJ, comprising
identifying, in a sample derived from a subject, the presence of at least one single nucleotide polymorphism in the Mucin-4 gene, gene product or a fragment thereof, thereby determining predisposition of said subject to develop ARONJ.

30. The method of claim 29, wherein identifying the presence of at least one single nucleotide polymorphism comprises applying next generation sequencing.

31. The method of claim 29, wherein said fragment thereof comprises SEQ ID NO: 49.

32. The method of claim 29, wherein said fragment thereof comprises SEQ ID NO: 50.

33. The method of claim 29, wherein said at least one single nucleotide polymorphism is selected from SNP Nos.: 101-147 and 151-156.

34. The method of claim 29, wherein said sample comprises blood, serum, plasma, urine, sweat, buccal smear, saliva or a combination thereof.

35. The method of claim 29, wherein said at least one single nucleotide polymorphism comprises a plurality of single nucleotide polymorphisms.

36. The method of claim 29, wherein said at least one single nucleotide polymorphism comprises at least 5 single nucleotide polymorphisms.

37. The method of claim 29 wherein said subject has a disease or disorder requiring anti-resorptive therapy.

38. The method of claim 37, wherein said disease or disorder is selected from the group consisting of: osteoporosis, osteitis deformans, osteogenesis imperfecta, bone metastasis, breast cancer, prostate cancer, lung cancer and multiple myeloma.

39. The method of claim 37, further comprising administering to said subject a composition comprising at least one anti-resorptive agent not associated with ARONJ or having a low probability of inducing ARONJ.

40. The method of claim 39, wherein said at least one anti-resorptive agent is selected from the group consisting of: bisphosphonate not associated with ARONJ, bisphosphonate having a low probability of inducing ARONJ, bisphosphonate at a low dosage, a non-bisphosphonate anti-resorptive agent having a low probability of inducing ARONJ, a non-bisphosphonate anti-resorptive agent not associated with ARONJ and a combination thereof.

41. The method of claim 40, wherein said bisphosphonate is selected from the group consisting of: clondronate, etidronate, tiludronate, risedronate, ibandronate and a combination thereof.

42. The method of claim 40, wherein said non-bisphosphonate is an inhibitor of cathepsin K.

43. A method of treating a disease or disorder requiring anti-resorptive therapy in a subject in need thereof, comprising:
identifying, in a sample derived from the subject, the presence of at least one single nucleotide polymorphism in the Mucin-4 gene, gene product or a fragment thereof; and
administering to said subject a composition comprising at least one anti-resorptive agent not associated with ARONJ or having a low probability of inducing ARONJ.

44. The method of claim 43, wherein treating comprises at least one of: preventing ARONJ development, inhibiting ARONJ development, suppressing ARONJ development and attenuating ARONJ manifestation.

45. The method of claim 43, wherein said fragment thereof comprises SEQ ID NO: 49.

46. The method of claim 43, wherein said fragment thereof comprises SEQ ID NO: 50.

47. The method of claim 43, wherein said at least one single nucleotide polymorphism is selected from SNP Nos.: 101-147 and 151-156.

48. The method of claim 29, wherein said at least one single nucleotide polymorphism comprises a plurality of single nucleotide polymorphisms.


SEQUENCE LISTING (57 sequences)

SEQ ID NO: 1 - DNA, 51 bp, Homo sapiens
gtgtcggtgacaggaagaggggtggcgtcacctgtggatgctgaggaagtg

SEQ ID NO: 2 - DNA, 51 bp, Homo sapiens
tcggtgacaggaagaggggtggtgtgacctgtggatgctgaggaagtgctg

SEQ ID NO: 3 - DNA, 51 bp, Homo sapiens
gaggggtggcctgacctgtggatgccgaggaagtgtcggtgacaggaagag

SEQ ID NO: 4 - DNA, 51 bp, Homo sapiens
aggaagtgtcggtgacaggaagaggggtggtgtcacctgtggatactgagg

SEQ ID NO: 5 - DNA, 51 bp, Homo sapiens
aagaggcgtggtgtcacctgtggatgctgaggaaaggctggtgagaggaag

SEQ ID NO: 6 - DNA, 51 bp, Homo sapiens
tcacctgtggatgctgaggaagtgccggtgacaggaacaggggtggcgtga

SEQ ID NO: 7 - DNA, 51 bp, Homo sapiens
gcgtgacctgtggatgctgaggaactgctggtgacaggaagagaggtggcg

SEQ ID NO: 8 - DNA, 51 bp, Homo sapiens
aagaggggtggcgtgacctgtggatactgaggaagcgtcggtgacaggaag

SEQ ID NO: 9 - DNA, 51 bp, Homo sapiens
gcgtgacctgtggatgctgaggaagtgtcggtgacaggaagaggggtggtg

SEQ ID NO: 10 - DNA, 51 bp, Homo sapiens
tgacctgtggatgctgaggaagcgttggtgacaggaagaggggtggtgtca

SEQ ID NO: 11 - DNA, 51 bp, Homo sapiens
ggatgctgaggaagcgtcggtgacaagaagaggggtggtgtcacctgtgga

SEQ ID NO: 12 - DNA, 51 bp, Homo sapiens
aagacgggtggtgtcacctgtggatactgaggaagtgtcggtgacaggaag

SEQ ID NO: 13 - DNA, 51 bp, Homo sapiens
gtgtcacctgtggatgctgaggaagggtcggtgacaggaagaggggtggcg

SEQ ID NO: 14 - DNA, 51 bp, Homo sapiens
gtcacctgtggatgctgaggaagtgacggtgacaggaagaggggtggcgtg

SEQ ID NO: 15 - DNA, 51 bp, Homo sapiens
tcacctgtggatgctgaggaagtgttggtgacaggaagaggggtggcgtga

SEQ ID NO: 16 - DNA, 51 bp, Homo sapiens
gtgtcacctgtggatactgaggaaaagctggtgacaggaagaggggtggcc

SEQ ID NO: 17 - DNA, 51 bp, Homo sapiens
gaggggtggcctgacctgtggatgctgaggaagcgtcggtgacaggaagag

SEQ ID NO: 18 - DNA, 51 bp, Homo sapiens
ggaagcgccggtgacaggaagagtggtggtgtcacctgtggatgctgagga

SEQ ID NO: 19 - DNA, 51 bp, Homo sapiens
tggtgacatgaagaggggtggtgtgtcctgtagatgctgaggaagggctgg

SEQ ID NO: 20 - DNA, 51 bp, Homo sapiens
catgaagaggggtggtgtgacctgtggatgctgaggaagggctggtgacag

SEQ ID NO: 21 - DNA, 51 bp, Homo sapiens
gtgacctgtagatgctgaggaagggttggtgacaggaagaggggtggtgtc

SEQ ID NO: 22 - DNA, 51 bp, Homo sapiens
actgaggaagtgtcggtgacaggcagaggggtggtgtcacctgtggatgct

SEQ ID NO: 23 - DNA, 51 bp, Homo sapiens
gtgtcacctgtggatgctgaggaagtgctggtgacatgaagaggggtggcg

SEQ ID NO: 24 - DNA, 51 bp, Homo sapiens
gtcacctgtggatgctgaggaagggttggtgacatgaagaggggtggcgtg

SEQ ID NO: 25 - DNA, 51 bp, Homo sapiens
tcacctgtggatgctgaggaagggccggtgacatgaagaggggtggcgtga

SEQ ID NO: 26 - DNA, 51 bp, Homo sapiens
ggatgctgaggaagggctggtgacaagaagaggggtggcgtgacctgtgga

SEQ ID NO: 27 - DNA, 51 bp, Homo sapiens
gggctggtgacatgaagaggggtggtgtgacctgtggatgctgaggaagcg

SEQ ID NO: 28 - DNA, 51 bp, Homo sapiens
ctggtgacatgaagaggggtggcgtcacctgtggatgctgaggaagcgtcg

SEQ ID NO: 29 - DNA, 51 bp, Homo sapiens
gtgacctgtggatgctgaggaagcgccggtgacaagaagaggagtggcgtg

SEQ ID NO: 30 - DNA, 51 bp, Homo sapiens
tgacctgtggatgctgaggaagcgttggtgacaagaagaggagtggcgtga

SEQ ID NO: 31 - DNA, 51 bp, Homo sapiens
ggatgctgaggaagtgtcggtgacaagaagagaggtggcatgaccggtgga

SEQ ID NO: 32 - DNA, 51 bp, Homo sapiens
gaggaagtgtcggtgacaggaagaggggtggcatgaccggtggatgctgag

SEQ ID NO: 33 - DNA, 51 bp, Homo sapiens
tgtcggtgacaggaagagaggtggcgtgaccggtggatgctgaggaagggc

SEQ ID NO: 34 - DNA, 51 bp, Homo sapiens
tgacaggaagagaggtggcatgacctgtggatgctgaggaagggctagtga

SEQ ID NO: 35 - DNA, 51 bp, Homo sapiens
aggaagggctagtgacaggaagaggggtggtgtcacctgtggatactgagg

SEQ ID NO: 36 - DNA, 51 bp, Homo sapiens
aagaggcgtggtgtcacctgtggatgctgaggaaaggctggtgacaggaag

SEQ ID NO: 37 - DNA, 51 bp, Homo sapiens
ggtgtcacctgtggatactgaggaagggctggtgacaggaagaggggtggc

SEQ ID NO: 38 - DNA, 51 bp, Homo sapiens
gtcacctgtggatactgaggaaaggttggtgacaggaagaggggtggcctg

SEQ ID NO: 39 - DNA, 51 bp, Homo sapiens
tcacctgtggatactgaggaaaggccggtgacaggaagaggggtggcctga

SEQ ID NO: 40 - DNA, 51 bp, Homo sapiens
ggatgctgaggaagcgtcggtgacaggaagaggggtggcgtgacctgtgga

SEQ ID NO: 41 - DNA, 51 bp, Homo sapiens
ggaagagaggtggcgtgacctgtggatactgaggaagcgtcggtgacagga

SEQ ID NO: 42 - DNA, 51 bp, Homo sapiens
cacctgtggatgctgaggaagggctggtgacaggaagaggcatggtgtcac

SEQ ID NO: 43 - DNA, 51 bp, Homo sapiens
aggaagggctagtgacaggaagagggatggtgtcacctgtggatgctgagg

SEQ ID NO: 44 - DNA, 51 bp, Homo sapiens
ggaagggctagtgacaggaagaggcgtggtgtcacctgtggatgctgagga

SEQ ID NO: 45 - DNA, 51 bp, Homo sapiens
ggtgtcacctgtggatgctgaggaaaggctagtgacaggaagaggcatggt

SEQ ID NO: 46 - DNA, 51 bp, Homo sapiens
aagaggggtggtgtgacctgtggatgctgaggaagtgtcggtgacaggaag

SEQ ID NO: 47 - DNA, 51 bp, Homo sapiens
agtgtcagtgacaggaagaggggtggtgtcacctgtggatgctgaggaaaa

SEQ ID NO: 48 - DNA, 65513 bp, Homo sapiens
tacagccccaaggtcgctccctctggggccctttcttccccattcttcccagcagcccaaagctctggtgggacaggggcagcccctggggagggaggagaggacccaggaacccggctaggagggtggcccacccatttccagtgtgacctgttcccattcccccatgtctcctcccatccctcccgccactcagctcaggctgatgagaagcagagcaacgggtgtatcggtgttttctttcctggtggggtagtggggtggggctgaggagagaaaagggtgattagcgtggggccccgccctcttttgtcctcttcccaggttccctggccccttcggagaaacgcacttggttcgggccagccgcctgaggggacgggctcacgtctgctcctcacactgcagctgctgggccgtggagcttccccagggagccagggggacttttgccgcagccatgaagggggcacgctggaggagggtcccctgggtgtccctgagctgcctgtgtctctgcctccttccgcatgtggtcccaggtaagtgatggagacagcagatgaggctggctgcggggagcacttgggggaggtgggagctgtcagagaaagaggtccggggagacagagagagagagagagagaataggggaaagggagacagcgaagaggaagagaagggagagaaaaagagggagagggaaaggagaaagagatgaatgggacaacatggggggaaggtggagagagacccagagagggaaagaagaggaagagaagagggagagagaaagaagagtggaggccgtgcgcggtggctcatgcctgtaatcccagcactttcggaggccaaggcaggagatcacctgaggtcaggagttcgagaccagcctggccgacatggtgaaaccccgtctctactaaatatacaaaaattagccggtcgtggtgggccccacctgtaattccagctactcaggagtctgaggcaggagaatcacttgaacctgggaggtggaggttgcagtgagccaagatcgcgccactgcactccagcctgggagagagagcgagactctgtctcaaaataaataaataaataaataaataaataaataaataaataaatataaaataaaataaaaaataagaagaggagaaaagtggggaagaggaggcatgaactggcagatacgggacaagatctgaggggaaagacagagggagaatgctcgaaagagagagaaaagagaacagagggccagagagcagcccggcgatgtctggaaggatccatggtgagagcccaggcttactcgcagagagaaagacaggcagagccagagcaagaggaacagagtcaaggagaaagatgtacacccttgtgtacagagctgggggtagaggggatgccaggaaagctgggtgatggagacggaagaaaactcatgtaaagctgcagggtgagaggacgagacaggtgagacgcagacaaactgaggaccctgggaatggagagaggagaagatcgggagacagcagcaagcaagggaagcgacaaggaggagaggggcaggccggccgggagggtggtgcggaggaggcggccagggcgcagagggccgggaggtgctggccgtgggcttcttacctctgagctcgggtttaaaagcctccatttgggtcacggccttgcctggggctcgtagccccggcattggccttgggctcctccgtgtacagagctgggaggggagggatgccaggcctgtgggagatgttccctcgggggcccccgtcctcttccccacactttccaggctgtccctctggcttcaggaccaagttttattctgtgtttctgggtgtctgagtctttgggggagagtctggggtccagagttcaagctggggttagagtctcagctcctgccctgcctctcagcaggctaagaacagtcgccgagggaaaatatttcttgggcgcatatttgaggagcttcctgggagtgagtcagaaggcgagtgccgtttaaaggctgcaagagaagccatgctggtgaagcggacccttccacctcgggatgtttcaggactaggctgagggcaaaggaaactgccaccacctccctacacctccccaccctccagcacccccaccccaccctggccacacaaccccgctccagtgctcatcccaccgtgaggacgtggaggccggaaggagccgccacacggccctgccctgcagatgtggttgaaggagtctccacgggaatcatgactcccagagcgaggctggggcttggggcgccggggaggcagcttggatttaggagccccagggccaagtctttgccgtgaactgttctggcccctgtgaccaggccctgccccgtgtctccccagggccccggtcccctgtgtaaaaagcagtggtgaacggttggacctcctgacgcccaagttcttgagtttccaaatctgtgatttaaagctgagcccaaatgtgctgggtaccagctggacactcagctccatgtggagccaggaagtggggtctgtggagaggagcgcagaggggcaagacctggggtgggcgtggaaaagcacgggggcgtgacccggagaaggagtgaaggactgttggtgtgcaagggcgtctccatgacgacccgaagaagctaggcatgtcgtggagcgctgagtcctttgcgtcgctaaggggaccaagtggagctgggccaggagaggagatggtcgtggctgggagatggcacccacacatctgaccgggcatgaccagggccttggcaggaaaagcagtcaccaagggcgggtgggcagcccccacccccacagggcagctgctggaggactggcagccagccagccccgttccttttggctccctgaaggggtttacagatgacctgcctatacttgagtctagggtctgtttgcacacttgccggcaggaccctcacccaggctgggtcacactgaagcccaggccagaggaaaaacacagggtttccacaaaggagctgccgcaatgagggtttccttaaggaacagccctggctctcaagggttaaaggataaggcacagcagacagaggtgggctagacaaggacagatggaaatttggtgtctactggtcgccccaggcaggaatgactcagaaggaagcctggccgtcctggttccatgccacagggaaaggcaactgggtcgaaataggccttggtctccagcactatcagtgaccccagggaggtgacaggctggagcaagtgcagggcaggcaggggaggggacgccggccacagcgcactccacggggaagggtctttatgggcccctcctcggagaacccccggtctatctgtcagtctgggacaggccacctcaacttgccaccgaggacaccaaaactctccacagacccctctgcccctctgggaaaccccactgtgctccaggacactcaaaaggaaaggatccctggacaagaggtcctgccaggaacatcagccaaattttggccaacgaccagcaaggtgcacagggaagagcaggggctgaaactcagaggtccagcatcagcgacgcccttggcagcccagggaacacaggcaacgccttttggctctggagtcttaggctcttcatcggcaaactgagcccagggggaaggggctactacgtagggttgtcatgaggatgaaacgagacagcatctggtgtaaagtagaaaaggcataaagggccgggcgcggtggctcacgctgtaatcccagcacttttggaggcccaggcgggtggatcacctgaggtcaggagttcaagaccagcttggccaaccctgtctccactaaaaataaaaaattttgccgggcgtggtggcgagcgcctgtaattccagctactcgggaggctgaggtaggagaatggcttgaacctgggaggcagaggttgcagggagccgaaatggcagcactctagcttgggtgacagagcaagactctgtctaaaaaaaaaagaaaagccataaagacgtgtttgagaaagaggcctgggaagacgggggaaggagggtgattgaacccggaatggcacttgtgtcggcccagggtcatatcccttcatctaaggatcctcgtgcctctaaaaagccaccccgtgcttcctgtgggtttgcaagggctggcttggtgtattcagaatgtggcttgctgcatgaacggaccccgagggccatggccctagagcaggggctcgctccagcggacagctctgcctcaccgctccctgcctgtgagtcccgccacgcccttggtttctgggctcagccgtggaggcagaggctggcctggcagaggctggcctggcagtgcttgacacgcaagtgatttgtgtcttcattgctaaggacaagaggcaatgagaggacaagaagtggttggccttttgtacgctcaacgggtggttttgctactctgtgtcttttctctgatttcacggtgctgttaagtgcttaaaatatgcacatcgtgtagctcacagagccacttctctgaaggccaggacagagaccttataggctctctctccccctagtttcagccttttaccttaaatatacgtctttcttactgctaggctgagttcccgccccagcatgttctgagaaattgagtcaaaataactgagtctgttggcacctcatcgacgatttcttcatagacggtttttttattgttgctgttgttgttggttttttgggtttgtttgtttgttttttgagacagagtttctctctgtcccccaggctgcagtgcagtggcgtggtctcagctcagtgcagcctctgcctcccgggttcaagagattctcctgcctcagcctcccgagtagctgggattatagacgcccaacaccacagcggctaatgtttgtatttttagtagagatggggtttcaccatgttggccaggctggtctcgaactcctgacctcaggtgatccgctcgcctcggctcccaaagtgctgggattataggcgtgagctactgtgcctggccctacttcatagaggtttaaatgccttttcacccttttcctggagactctgaagaagtctcaggaactgggcatttgtgttgcacgtgaggccttgcaatggcggccctgcttggaggaagggcactggcctgggttgcccgcagctccactccccgtgtatgtgtttagggaccacagaggacagacatcgactctctgtagagatgccgccccgcccaggttgcagtttaggttccaaaagtccagtggccagtggattttgggggaatttggaataagaaacagcctagactttggagttgttcattcacttgcagaatttctactcatgccagctgctctggacaggaagatgaatgcgtcacagttcctgcttttcaaagctctctaagttaagtgacttgtttaagatcatagaacccataagtgaggcagctgggactagaacccaggtctcctgactcactgcagcacacagcctttcggcaatctccaaaccagcccagcccaccgacggagggaagaacagaagcattcacacaccctgctgagacagccattcattcattcatttgttaattaaaccaccatttaggaaacgcctgccttaagttcctgacattgttctaggacacagcactggatgcacacagtgaagagtgaaacagacgtggcccagtctcttggcactaaaatcttggtgcagacagacatcaaataattacggaaatgttctcaactgcacatgtggtaaatgcagtgtggaaaagtacagggtgtgctgagagctgcatttcgaatggccagagagtaggggaggtgcatctgactgacaagtcaggaagggccctgtgaggaaccgttctgcggggagctgaggcctgaggctgaggacagccaggtggagaaggtgccaggcctgagcaggcagaggcggagctcatggagaggcaggaaagagcttggccccttggaggacttgaaagagaaggcaggtgttagatcgcgcagggctttgcaagctgtggaatagattatgaattttatgttattttatttatttatatttttgagacagagtctcactgtgtcacccaggctgaagtgctgtggcgttatctcggctcactgcaacctctgcctccagggttcaagggattctcgtgcctcagccccccgagtagctgggatcacaggcacatgccaccatgctctgctgatttttgtatttttagtagaaatggggttttgccatgttggtcaggctagtctcgaactcctgacctcaagtgatctgcttaccttggcctcccaaagtgctgggattaccagtgtgagccactgcgcccggcccagattatggattttgagtaattgaaatgacagacaaggccaggcacagtgactcatggctgtcatcccagcactctgggaagctgaagcgggcagatcacttgaggtcaggagtttgagaccagactggccaacatggcaaaaccccgtctctactaaaaatacaaaaattagcaggagatggtggtgggcacctgtaatcccagctactcaggaggctgaggcaggagaagcacttgattccaactgggatcacaggcacacaccaccacaccaggacagcccgaggctaaccactgtggagaggcagccaggctcctgaggacagcgctaggctaggaggatgtgcctgccccagtgggagccaggagagatgggtggcaacaccaagatggtggagactggagatggcacctccgagaaggaggcgttggggaaaagagagttgagctggccggcgtgctgtgctcacagccggattttggtgcttcttggtttggtgctggagcttctgtggcgatcaccagagtggctgggtgctgccacaccagatgctgacagaaatgaggtcaaatgaccggtggtgggatccatgggttggggtgtgacttcggcggggatcgttggaaagaacgggtaaagttctcgccttggagtccactgtgaagggacttgaacctgggaggtggaggttacagtgagctgagatcgcaccactgcactcctgcctgggcgacagagcgagactccatctcaaaaaaaaaaaagataaagataaagaaacaggcaacgtcaccctctctccagcctcggctgaaggcaacaaaggccacagggcctgggatgtattccacagatgtgtacgaagtcactgctgtatgccaagtctcgtttcaggcgctggagatcacaagaggtctgttattccaagggtatcttgagttagtgtctttcctcatcttgaattctagggaagtacccagtggacacaaccctggtcacagttgattctgggctgatacacaaatagcattagctccctgccaaaggcggtggggccagcggttcagcgtctgcgcgccttccgggccgcctgctgcctgtgtggggctgaatgttctcctcaaggccatccttaccgttgggcacttcccttgggcttacactgccctgcccgaggcagcctggcaaggggccccacccgtcaggaagtaattcccctccaggaaagtgaaacactgtggcgatacggcttacgggttcctcacctccagttccaggaaaaacccaagacaaacagaccagatgctaggaataagcctcaacccacagcacttagaaagccgcaggctttgtgtgtcttcagggagtggcttcacccttcttggaatctgtcttctgccagcgtcactcatactgctgccactccttctcccgagccaacagcacacctgtcacctcggcacacctgtggagtcaccagaggggccatctggggaaaacgatgcctactgccactcggccttcacatcctggagccctaacagacgtgaggcagctgtgggccgtctcccacctgacgtggttcaaggagagaaaagaactaccttgatgggagatctctggctgacgtgatggcagaggcttgacagagccttcaaggactcagatgtggggaacgtctctacctcagtgccaacgccagtgccctcctcgtccctggatggagactcttcctctgagagaatgtcttaaaaactatccgttttactcaatgacacacgtgtgtcttctcacagtgatcttgtcaccttaaggtgtcaacagccttcacactcactctaacatcatcccagaaaagagacaccaggaacattgcaaaaacaccccagtgtcacagcgtccaccccgagaatgtgcctggctcggcctcaggctcagagccctctgcatgtggcagtccaagaacttggggcaggtcagtgaccatttctccatctttgcctcagctctccatccactcggtggcagtcccagagctgagcagctcgcagcttccgagcaatggctcttaccccgagcccccggccctgctacttccaaggcatataatatcactcggtctcttcaaatctcaatttccctgcctgtgaaatggggccgctgaatctcgatttccccgcctgtgaaatggggccgctattaacaagataagctataaagcaccacatgaacatgagctgtgaacatttgtattattcatagagtcataacgtgcctacgagagaagtaaaaagaagccccacctccctggtgtgttcatcacaagtcagctgaccctcagtcactcgacccagcgcagatgcggaggctctcgctgcttgcagagagtttatattatattaaacggatagtttaattcataaggtctggtaggaagaacgtgaatttattaaccaaaactagtaaaggggaagcagccagatttctacttaaagtagctgctttaattttcagaggggaaagccttgataaggaaggcacgcaagaatcgggctgagtcctgtgtccgcatgtcttgttctggtggctcttctgggtcccagtccgtccggacctctggctgacattattttaacaagagctgtcttgaagtaatttctgggattttgcagctggattttcagatgtggtttgtctctctcaaggttttagcccctgcaacttttaggaagacatataattcgatcctcgcacgcagagaaatggaagggagtatatacagacagtaagaaaagggagggagtgtatacagatgacaagaaaagggagggagtgtgtacagacggtgagaagagggagggagtgtgtacagatcgtgagaaaagggaggggcgtgaagtctatttttaggctgaggaaaacggcctttgcagctgcctcaaggttgtatttggaaacccaagagaaaggggaaaacatgtttaaaatgcattttgaatttaagctgccggctacaaaactgaacaatgtctcccgcagaaacgagaaatcccttggcgtggccatttcttattacagaagccaccctgccacgcacacagacgcgctggaagtatctttcctcagtcacaaccagacgaaggtcattcttgcccagtggcctccctctgaggattctggtttctgacatccgtgtcacaatggcaatgccagtcaccagtctctgaccactagacccacgcccccgggacctacgcagacacccaggaccaatgccagtcaccagtctgtgaccccaggacctaccagacaccttccagggcaggcagggcaggccattttccaaagcaattcgagctaaggagacagcagcagcaaccctccctcctggccctcagagctgaggatcaacccacaggggtgcggtggaggctgagagcaggggtgcggaacccgagtcagcaggggcagtgaggaggctgccgcagtagtcaaagcgacggggaggatgtggcaaagcaaacaagaggacgcgatggggtgaacacttgggaggaagatcaacgagtactgactaggacacaggggacaaggaagagaaagaaatccagaggacccacagctttctgggctggatgtctgggggactgttgttagcatgaacagaaggaaggaagaaagcttgcaaggaaagattgacctggacctgttgcattgcatgagctaggcaggaaatccctgggagggtgaggccagcatatgggtgggaagtcagggagaagtacagatttgtaaggcgtgagagtagacgcagggtggccagagagggaggggcagcaggagagggaaagggctggagacatgtcatctgggcgtggagcgggggagagaggtggcaaggggagtggagtggagggcccgggagcgtggacggtggagcaggacccgtcagcttcaatgagggtgaggctgggtggtggcacggggccgccggccagggagagcacgtggaggggggggcgcaatacacatctgtcagattcatgaatgaatgaatggatggggccgaactgatgagacgcgtgaggactggagaaaatcgtttttgctgttgggaaggagattctggtagctctgaggaaaacggcttgagcgaagtgagtgaggtgggggcagagcgagcagtgcgtggtaaagaagaggcggcaggggaagcccggctttcatgctgctggagggacgaggaggacacagacggatgacagacttgttttaggccaacagacacctgaacatgctcacaggcaggagagaaggagaaaggcagaaagaacgtcggagacacagaggcagagcagtgagccatccgggcaggagggagccccggggccccggggagggtgcggagaaggctctgggcattgaaggagatgaagggcacggctttggttctttcagtgaagtagaaaatggagggtcacggggagagaagagagaggagagacctgtttctctccaatatcaactctttaggcgcaaatctcccagctctctgctccccaagtttatgcgtttggccagagtgagaacaagtttcagttgtctgacaatctgacggaacctgaagggcctgacttgcatcctggtgagagccaaggtcagttcattctggccaacacttgagcagcaagaacaaggctatgagtattttttagaataaaacttgatcaaggcttcaaatttctctgcctgacaacttcagcgagatggttttggcttacaaacattgttaagagtggatactatcattttattttaagggaagtgtttctgaacgtggggtccacggaccacctgcatcagaatcactctggacatgtgttaaaatgctgatgcaccctctgccttttgagaaccactttttttttttttttttttttcagagaaggggtcttgagctattgcccaggctggtctcaaactcctggcctcaaggaatcctcccgcctctgcctgtccgtgctgggattttaggcatgagctaccatgcccagctgagaaccattttcttaccaaaaatcattctccatagtctctccatccactaggatttctttctttttcttctttgtaaagataggtcatacttgaacacaggatttcttacctacaaataacctactcaagcttagtagattgagttatctgcaaattgttttcctaaacagctgaataatcaatctattgcaggagtggcttcagaggtattgtttaaatgatggtgaaagacaaatgcaataactgtagttttgtcgtaacgaatgctaatctcttcctcctcacaagcccaacagattgagagattttgacagcgccttctcccctagacaataaaactatgtcctccacatgcagtgactcacttgccctcctatttgcggcagcaggaaactctaatcaacttggctcaagatgagcaagttctctaaataaagatttaaaaggaggactaccaggacacagccctgtttagcaagaattttttttttttttgagatggagtcccgctctgtcgcccaggctggagtgcagtggcgcgatctcggctcactgcaacctccacctcccaggtttgagagattctcctgcctcagcctcctgagtagctgggattacaggtgcacgccaccatgcctgggtaatttttgtatttttagtagagatggggtttcatcatgttggccaggctggtcttgaactcctgacctcaaatgatccacccgcttcgacctcccaaagtgctgggattacaggtgtgaggcaccgcacctggccgattttcagtcgatgctgtgaagcaaagagcagcaggtgagggatggggcctgtcctggatcatgcccctttgttcacaggctagggatatggggaaacaggcacactcttgcacactgccaacaggtctgtaaattggcataagctttctgcaaagaaatttggcagtctgtattaataacatgaaaaattttataacggcataaaccagcaattccactgatagaaatttatgttaaggagatgagctgaaataatgtaaacaaaacattagacacgaagatagtcatcagggctttatttaaaataagcaatacattggaaatatcctaattgtccaacagtaagttaacaatatgagctttctctatgatggcttattatttagtgtttaggccgggcgcggtggctcacgcctgtaatcccagcactttgggaggctgaggtgagtggatcacctgaggtcaagagtttgagaccagcctggccaacatggtgaaacccagtctccactaaaaatacaaaaaattagcctggcatggtggcacatgcctgtaatcccagctacttgggaggctgaagcaggagaatcacggaggtggaggttgcagtgggccgaaatcgtgccgttgcactccagcctgggcaacaagagtgaaactccatctggaaaaaaaaatgtttaaaggctatgtccaatgttccataagggcagggacacacacacacagtgtcacacacacacacacacacacacacacacacagtgtcacacatacatactagcaacacacagaacgggcataaagtaagatgtattaaggagaaattgcgagcagggagcagtggagtccacatgacccccgagctctcattgcacagagaggtgggtatggtggacacacagcaggatgtggtacaggagagagacctgctcctgtttaacagaaaatgggaagggtaacaacgcttccagttgggggagtggctttgttaggaaggaggccaccagagctcagcagggggagtttttatacagcaccctagattttactatggagctgagctctcccagtccccctgagccagctcttgtaggactccaaaagggggaagcaaggaactgccatccaagacataaaagatgtgcgtcttctcaggctatgtttacgaagctagtatcttgaagaaatgctcatatcaggtatggctaaaaaggcaggatacaaaattatgtatactgaataagttaaactatacatatatattttttaatttttaattttttttttgagacggagtctcactctgtcacccaggcaggagtgcagtggtgcaatctcggctcactgcaagctccgcctcccgggttcacgccattctcctgcctcagcctcccgagtagctgggactacaggcgcccgccaccacgcccggctaatttttttgtatttttagtagagacgggggttcaccctgttagccaggatggtctcgatctcctgacctcatgatccgcccacctcagcctcacaaagcgctgggattacaggtgtgagccaccacgcctggcctaaactatatattttttaaagcataaaaacatcctggaaggaagcattgtctggtagcaagattatgggttttgatctttcaactataaatgttcccaaatgcacgtaaccagtatgtgtgttttctttggcatatttcaatgcagttgtacaattttgtccataaagattaagcacatcttttgttagatttatttttatgtactttttttggtcaccatcataaacactatagtcttaaaaatattttctctttcactgatatataggaatgcagttttattttgtttttaaatctctttttgagacagagtctccctctgtcatgcaggctggagtacagtggggtgatcatagctcactgcagcctcgaactcttgggctcaagtgatcctcccccctcaacttctgagtagctggaactatatgtgtgtgctactatgcccagctaattttttatttttttattttttatttctgtagagacaggatcttgctatgttacccaggctggtcttgaactcctggcctcaagtgttcctcctgcctcagcctctcaaagcactgggattcaggcatgaatcaccacacccagctggaatgcaattatttttaaaattttgattatggatatagaactttgctaataatgtatatgtttgggaagtttctatgtaaacaattatgttatctgcaaataaccataggcttgtttcatcccttccatacccaaaatcacatttctgtactctcatactttttgtttaattttctcgtcttatttcaccagctaaaaccttcaacctaatgttgattagaagcagtgattgttggcatcttgtttttattcctgattttacagaaacgtttctattgttttaagtataatgtttacacggggttataattgacattgtttatcaggttaaaagagttaacgtctctttttagtttgctaggagttttaacaggagtaagtgttgacttttatcaaataactttcctgtatctgctgtggtgattgtgtctcccctccgttctgtttatatggtgatttacattaagaaagcattgtctttatcttgcatttctgatttaaactcagcttgatcatgatgtacttttaaaatatttatttatttatttatttatttaagatagagtcctgctctgtcaccaggctggagtgcagtggcacaatctcggctcactgcaatctctgcctcctgggttcaagcaattctcgtgcctcagcctcctgagtagctgggattacaggtgtgtgccaccaagcccagcacacacctgtaatccaaaaaaatacaaattttttgtatttttaccagagacggggtttctctaggttggccaggctggtctggaactcccggcctcaagtgatccgcctgcctcggcctgccaaagtgttggggttacaggcgtgagccgccgcgcccggccatgatgcactttggcttgctaatattttatttacgatgcttccatgtattattactgggacttattaatctatagtttgtctttcttatcctatctttggtttccaaccttataaaattattagtagatcatttcctcctgttccaaactctgaatatgtttgtatagaattgaaattacctgttccttgaatgtctgatttgcctgtaaactcatcttagcctgcatttttttgtggggtggatagattatatatacatatttttttttgagacggagtctcaccttgtcacccaggctggagtgcaatggcatgatctccgctcactgcaacctctgcctcccaggttcacgccgttctcctgcctcagcctccagagtagctgggactacaggcgcctgccaccatgcccagctaatttttttttcaccgtgttagccaggatggtctcaatctcctgacctcatgatccgcctgcctcaccctcccaacgtgttaggattacaggcgtgagccaccgcacccggcctagattatattttttaaactgctgattcaatttaaaatagtttatagtggccgggcgaggtggctcatgcctgtaatcccagcactttgggatgccgaggtgggtagatcacctgaggtcaggagtttgagaccagccaggacgacatggtgaaacccagtctctactaaaaatacaaaaattagccgggcgtggtggcatgcgcctgtaatcccagctacttgggaggctgaagcaggagaatcttgaacctgggaggcagaggttgcagtgagctgagatcatgccattgcactccagcctgggcaacaaagcaagactctgtttcaaaaaaataaataaataaaattaaataaataaaataaaatagtttttagccaggtttttttcttaattcatttttgataaattatatttttaaggacagtatatccaatttatctaagttttcatactttttggtataaattcatcagagcactgtctcttaaattttttttctgaagttatgtacccttttttgtttctaataatcttgatatctgtgtcttttctgtctctctctctccctcctcccacccccctcccccactttctttctcttgccaggtatttgttttattttcaaagaactaatctttggcttcatttattccatttttaattgtttctatagtgagttctggtctgttactatatccttcattctgcttcctttttcttttttcttcctggtctttctagtctaccttttttgctggcggttccagctttttgcagggaaactcagttcagtgtcgtgaccgagggtcttgcttcctttcctttttcagcctttcagtcagcacatttccaagggcattgactcaccatcttagttgttaacttcttcactcctgagtgtttccctttctttcttctcatctcacctctgcattagaaaacatttattacatttataacctgtatttatctggcattggctggttttgctattagaggcttatctcttcttccatattgctgaaatggagctgtgccacttttataaagaactacaaaaatctttactttaaaaaaaaggacggtgctgtcatgaaacttctgctgctgacactgattttagcagaggtgatgcaggctctgaaaagatgttaattgccaagaatttacacattcagaaagtttggcccctagatagctcctgtctactcaaagcctgatgatagacaaatgctcttaattatgttatcagggaaatgaaataaggttaaaaataactgcatagagaatgtttttaaatttaaaccttatttggagaaaccaagttttgcaaattttctctaggagaaagcaggcatcgagtttagagtcctatcaacaaatctcaacctaattccaactaaaggaagtttccgtttaaagttgaagtggagtcattttggattaagccaggggcccagggacctcccagagggctctgcggttcagcccgtgcctttgtccacgtcggtttacaagtgtgtcaccagtaagcagttactattaatgaagtgcagaagcttgcacgtgagtcacttttttaacaattagagctggttcataaccattatcactcacttgagagtctaagttactgtctgaagaagagaaggtggcgaattacagtcaattccagaaagtggacttgaagctgaattcttccctgagactttgtctctctgagtatcatcttgccaactatctaggagcactgaggttgaaaaaaagggtgggagcggtggctcacacctttaatcccagtactttgagagtctgaagcaggaggatcgcttgaggccgggagttcgacaccagcctaggcaacatagcaagattccacctctacagaaataaaaaaattacccaggcatggttgtgtgtgcctgtagtcccagctactcagaaggccaattacaccgctgaacttcagcctgggtgacagaagaccctgtctctctttctctctctctctttctttctctctctctctctttcttctctctctctctctctctatatatatatgtttaaaaagaaaaaagaaagaagtttgagaacagctagggaactaatgaaaacccaaaagagtcagtttctaagagaaaaaaaaaggcaagttttcttggtttcttcttggttcagagactcagaggctaaggtaatattcacgaagatggtatgtggctagtgcaggtgctgtgaaatattttaaacaaaaaaagccaatatttaaacattctgcctgggaaatatttttctttatttatttaatatctatttttattattattattattatttttattttttaagacacagggtctcgctatcttgcccaggctggtctcaaactcctggcctcgagtgatcctcccaccttggcctcccaaaatgctgggattacagacatgagccaccatgcctggcctctgcctgggaaataatttttttttaaaaagtggttatggtcaataatattagcttcttggtagtctgtgaccagggcaagagtatatgcatttaccccttgggttttttaccagattggcacagatgttaacaacgaaaaagaaaagtagaaatttaatttatttttaagaaagataatgctataattcaagttgagagagttaacccggcttatagcaggggtactgtttgcctcttcccatggagtttaacacagttaattcaccattctagtctaacctcaccttcctcctcacaaggaagacaggcgggagctggagaagaccattggctatattcctgcaactgtgctttttctaggcgatttgaagcagtgcttatcaggggccttcctctgtgggaatcctgcacccccatctctctctccttttttattttatttatttattttttttttgagacagaatcttgctctgtcgcccaggctgcagtgaagtggcatgatgtctgctcactgcaacctccacctcctgggttcaagcaattctcctgcctcagccttttgagtaactgggattacaggcacccaccaccacgcccagctatttctgtatttttttttctagtagagacggggttttgccatattgcccaggctggtctcgcactcctgacctcaagtgatccacccatctcagcctcccaaagtgctgggattgcaggtggggctaccatgcccggccccatctcttattatttttttatcccccaaatatgtacagttgtgatatattaatttttcattttttctttttatttcttttcatttttactttaagtttcaggatacatgtgcagaacgtgcaggtttgttacacaggtatatatgtgccatggtggtttactgcacctatcaacccatcacctgcatgtattagctatttatcctgacgctcttcctccccttgtcccgtccctgacaggcgccagtgtgtgttgttcccctctctgtgtccatgtgttctcattgttcaactccaacttatgagtgaaaacatgcagtgtttggttttctgttcccctgttagtttgttgaggatgatggctttcagcttcatccatgtacctgcaaaggacgtgatcccattcctttttcatggctgcatagtattccgtgctgcatatgtaccacattttctttatccagtctatcactgatggacatttgggttggtttcatgtctttactattgtaaatagtgctgcagtaaacatacgtgtgcatgtatctttataacagaatgatttatattcctttgggcatatacccagtaatgggattgctgggtcaaatggtatttctggttctagatccttgaggaatcgccacactgtcttccacaatggttgaactgatttacactcccaccaacagtgtaaaagcgttcctatttcaccacagccttgccagcatctgctgtttcttgactttttaataattgccattctgagtggtgtgagatgatatccctttgtagttttgatttgcatttctctaatgatccatgatgatgagccttttttcatatgtttgttggtggcataaatgtcttcttttgataagtgtctgttcatatcctttgcctgctttttgatggggttgtttatttttttcttgtaaatttaagttccttgtaaattctggatattagacctctgtcagatggttagattgcaaaaattttctcattttgtaggttgcctgtttgctctgatgatagtttcttttgctgtgcagagctctttagtttaattagatcccatttgtcaattttagcttttgttgcaagtgcttttggagatttcatcataaaatctttgcctatgtctatgtcctgaatggtattgcctaggttttcttctagggttttcatggtttggggttttacatgtaagtctttaacctgccttgagttaatttttgtataaggtgtaaggaaggggtccagtttcagttttctgcatatggctaaccagttttcccagcaccatttattgaatagagaatcctttccccattgcctgcttttgtcaggtttgtcaaagatcagatggttgtagatgtgtggtcttatttccaaggtatctattctgttccattggtttatatgtcagttttggtaccagtaccatgctgttttggttactgtagccttgtagtatagtttgaagtcaggtagcctcccactttgttctttttgcttaggattgtcttggctatttgggttcttttttgattccatatgaattttaaagtagtttgttctcattctgtgaagaatggtagttttcacatcctttgttagctgtattcctaggtattttattctatttgtagcaattgtgaataggagttcattcatgatttagctctctgcttgcctattttggttcactccaggaaccacagaggacacattaataactggaagtaaaactgctgccccagtcacctcaacaggctcaacaacagcgacactagagggacaatcaactgcagcttcttcaaggacctctaatcaggacatatcagcttcatctcagaaccaccagactaagagcacggagaccaccagcaaagctcaaaccgacaccctcacgcagatgatgacatcaactcttttttcttccccaagtgtacacaatgtgatggagacagctcctccagatgaaatgaccacatcatttccctccagtgtcaccaacacactcatgatgacatcaaagactataacaatgacaacctccacagactccactcttggaaacacagaagagacatcaacagcaggaactgaaagttctaccccagtgacctcagcagtctcaataacagctggacaggaaggacaatcacgaacaacttcctggaggacctctatccaagacacatcagcttcttctcagaaccactggactcggagcacgcagaccaccagggaatctcaaaccagcaccctaacacacagaaccacttcaactccttctttctctccaagtgtacacaatgtgacagggactgtttctcagaagacatctccttcaggtgaaacagctacctcatccctctgtagtgtcacaaacacatccatgatgacatcagagaagataacagtgacaacctccacaggctccactcttggaaacccaggggagacatcatcagtacctgttactggaagtcttatgccagtcacctcagcagccttagtaacatttgatccagaaggacaatcaccagcaactttctcaaggacttctactcaggacacaacagctttttctaagaaccaccagactcagagcgtggagaccaccagagtatctcaaatcaacaccctcaacaccctcacaccggttacaacatcaactgttttatcctcaccaagtggattcaacccaagtggaacagtttctcaggagacattcccttctggtgaaacaaccacctcatccccttccagtgtcagcaatacattcctggtaacatcaaaggtgttcagaatgccaacctccagagactctactcttggaaacacagaggagacatcactatctgtaagtggaaccatttctgcaatcacttccaaagtttcaaccatatggtggtcagacactctgtcaacagcactctcccccagttctctacctccaaaaatatccacagctttccacacccagcagagtgaaggtgcagagaccacaggacggcctcatgagaggagctcattctctccaggtgtgtctcaagaaatatttactctacatgaaacaacaacatggccttcctcattctccagcaaaggccacacaacttggtcacaaacagaactgccctcaacatcaacaggtgctgccactaggcttgtcacaggaaatccatctacagggacagctggcactattccaagggtcccctctaaggtctcagcaataggggaaccaggagagcccaccacatactcctcccacagcacaactctcccaaaaacaacaggggcaggcgcccagacacaatggacacaagaaacggggaccactggagaggctcttctcagcagcccaagctacagtgtgactcagatgataaaaacggccacatccccatcttcttcacctatgctggatagacacacatcccaacaaattacaacggcaccatcaacaaatcattcaacaatacattccacaagcacctctcctcaggaatcaccagctgtttcccaaaggggtcacactcaagccccgcagaccacacaagaatcacaaaccacgaggtccgtctcccccatgactgacaccaagacagtcaccaccccaggttcttccttcacagccagtgggcactcgccctcagaaattgttcctcaggacgcacccaccataagtgcagcaacaacctttgccccagctcccaccggggatggtcacacaacccaggccccgaccacagcactgcaggcagcacccagcagccatgatgccaccctggggccctcaggaggcacgtcactttccaaaacaggtgcccttactctggccaactctgtagtgtcaacaccagggggcccagaaggacaatggacatcagcctctgccagcacctcacctgacacagcagcagccatgacccatacccaccaggctgagagcacagaggcctctggacaaacacagaccagcgaaccggcctcctcagggtcacgaaccacctcagcgggcacagctaccccttcctcatccggggcgagtggcacaacaccttcaggaagcgaaggaatatccacctcaggagagacgacaaggttttcatcaaacccctccagggacagtcacacaacccagtcaacaaccgaattgctgtccgcctcagccagtcatggtgccatcccagtaagcacaggaatggcgtcttcgatcgtccccggcacctttcatcccaccctctctgaggcctccactgcagggagaccgacaggacagtcaagcccaacttctcccagtgcctctcctcaggagacagccgccatttcccggatggcccagactcagaggacaagaaccagcagagggtctgacactatcagcctggcgtcccaggcaaccgacaccttctcaacagtcccacccacacctccatcgatcacatccactgggcttacatctccacaaaccgagacccacactctgtcaccttcagggtctggtaaaaccttcaccacggccctcatcagcaacgccacccctcttcctgtcacctacgcttcctcggcatccacaggtcacaccacccctcttcatgtcaccgatgcttcctcagtatccacaggtcacgccacccctcttcctgtcaccagcccttcctcagtatccacaggtcacaccacccctcttcctgtcaccgacacttcctcagaatccacaggtcacgtcacccctcttcctgtcaccagcttttcctcagcatccacaggtgacagcacccctcttcctgtcactgacacttcctcagcatccacaggtcacgtcacccctcttcctgtcaccagcctttcctcagcatccacaggtgacaccacccctcttcctgtcactgacacttcctcagcatccacaggtcacgccacctctcttcctgtcaccgacacttcctcagtatccacaggtcacaccacccctcttcctgtcaccgacacttcctcagcatccacaggtcacgccacctctcttcctgtcaccgacacttcctcagtatccacaggtcacaccacccctcttcatgtcactgatgcttcctcagcatccacaggtcaggccacccctcttcctgtcaccagcctttcctcagtatccacaggtgacaccacgcctcttcctgtcactagcccttcctcagcatccacaggtcacgccacccctcttcttgtcaccgacacttcctcagcatccacaggacacgccacccctcttcctgtcaccgacgcttcctcagtgtccacagatcacgccacctctcttcctgtaaccatcccttccgcagcatccacaggtcacaccacccctcttcctgtcaccgacacttcctcagcatccacaggtcaggccacctctcttcttgtcaccgacacttcctcagtatccacaggtgacaccacgcctcttcctgtcactagcacttcctcagcatccacaggtcacgtcactcctcttcatgtcaccagcccttcctcagcatccacaggtcacgccacccctcttcctgtcaccagcctttcctcagcatccacaggtgacaccatgcctcttcctgtcactagcccttcctcagcatccacaggtgacaccacccctcttcctgtcaccgacgcttcctcagtatccacaggtcacaccacccctcttcatgtcactgatgcttcctcagcatccacaggtcaggccacccctcttcctgtcaccagcctttcctcagtatccacaggtgacaccacgcctcttcctgtcactagcccttcctcagcatccacaggtcacgccacccctcttcttgtcaccgacacttcctcagcatccacaggacacgccacccctcttcctgtcaccgacgcttcctcagtgtccacagatcacgccacctctcttcctgtaaccatcccttccgcagcatccacaggtcacaccacccctcttcctgtcaccgacacttcctcagcatccacaggtcaggccacctctcttcttgtcaccgacacttcctcagtatccacaggtgacaccacgcctcttcctgtcactagcacttcctcagcatccacaggtcacgtcactcctcttcatgtcaccagcccttcctcagcatccacaggtcacgccacccctcttcctgtcaccagcctttcctcagcatccacaggtgacaccatgcctcttcctgtcactagcccttcctcagcatccacaggtgacaccacccctcttcctgtcaccgacgcttcctcagtatccacaggtcacaccacccctcttcctgtcaccagcccttcctcagcatctacaggtcacaccacccctcttcctgtcaccgacacttcctcagcatccaaaggtgacaccacccctcttcctgtcaccagcccttcctcagcatctacaggtcacaccacccctcttcctgtcaccgacacttcctcagcatccacaggtgacaccacccctcttcctgtcaccaatgcttcctcattatccacaggtcacgccacccctcttcatgtcaccagcccttcctcagcatccacaggtcacgccacccctcttcctgtcaccagcacttcctcagcatccaccggtcacgccacccctcttcctgtcaccggcctttcctcagctaccacagatgacaccacccgtcttcctgtcaccgacgtttcctcggcatccacaggtcaggccacccctcttcctgtcaccagcctttcctcagtatccacaggtgacaccacgcctcttcctgtcactagcccttcctcagcatccacaggtcacgccagccctcttcttgtcactgacgcttcctcagcatccacaggtcaggccacccctcttcctgtcaccgacacttcctcagtatccacagctcacgccaccccacttcctgtcaccggcctttcttcagcttccacagatgacaccacccgtcttcctgtcaccgacgtttcctcggcatccacaggtcaggccatccctcttcctgtcaccagcccttcctcagcatccacaggtgacaccacccctcttcctgtcaccgacgcttcctcagcatccacaggtgacaccacctctcttcctgtcaccatcccttcctcagcatcttcaggtcacaccacctctcttcctgtcaccgacgcttcctcagtgtccacaggtcacgccacctctcttcttgtcaccgacgcttcctcagtatccacaggtgacaccacccctcttcctgtcaccgacactaactcagcatccacaggtgacaccacccctcttcatgtcaccgacgcttcctcagtatccacaggtcacgccacctctcttcctgtcaccagcctttcctcagcatccacaggtgacaccacgcctcttcctgtcactagcccttcctcagcatcctcaggtcacaccacccctcttcctgtcaccgacgcttcctcagtacccacaggtcacgccacctctcttcctgtcaccgacgcttcctcagtgtccacaggtcacgccacccctcttcctgtcaccgacgcttcctcagtgtccacaggtcatgccacccctcttccggtcaccgacacttcctcagtatctacaggacaggccacccctcttcctgtcaccagcctttcctcagcatccactggtgacaccacgccgcttcctgtcaccgatacttcctcagcatccacaggtcaggacacccctcttcctgtcaccagcctttcctcagtatccacaggtgacaccacgcctcttcctgtcactaacccttcctcagcatccacaggtcacgccacccctcttcttgtcaccgacgcttcctcaatatccacaggtcacgccacctctcttcttgtcaccgacgcttcctcagtatccacaggtcacgccaccgctcttcatgacaccgatgcttcctcattatccacaggggacaccacccctcttcctgtcaccagcccttcctcaacatccacaggtgacaccacccctcttcctgtcaccgaaacttcctcagtatccacaggtcacgccacctctcttcctgtcaccgacacttcctcagcatccacaggtcacgccacctctcttcctgtcaccgacacttcctcagcatccacaggtcacgccacccctcttcctgtcaccgacacttcctcagcatccacaggtcaggccacccctcttcctgtcaccagcccttcctcagcatccacaggtcacgccatccctcttcttgtcaccgacacttcctcagcatccacaggacaggccacccctcttcctgtcaccagcctttcctcagcatccacaggtgacaccacccctcttcctgtcaccgacgcttcctcagtgtccacaggtcacgccacctctcttcctgtcaccagcctttcctcagtatccacaggtgacaccactcctcttcctgtcactagcccttcctcagcatccacaggtcacgccacccctcttcatgtcaccgacgcttcctcagcatccacaggtcacgccacccctcttcctgtcaccagcctttcctcagcatccacaggtgacaccacgcctcttcctgtcactagcccttcctcagcatccacaggtcacgccacccctcttcatgtcaccgacgcttcctcagtatccacaggtgacaccacccctcttcctgtcaccagctcttcctcagcatcctcaggtcacaccacccctcttcctgtcaccgacgcttcctcagcatccacaggtgacaccacccctcttcctgtcaccgacacttcctcagcatccacaggtcacgccacccatcttcctgtcaccggcctttcctcagcttccacaggtgacaccacccgtcttcctgtcaccaacgtttcctcggcatccacaggtcatgccacccctcttcctgtcaccagcacttcctcagcatccacaggtgacaccacccctcttcctggcaccgacacttcctcagtatccacaggtcacaccacccctcttcttgtcaccgacgcttcgtcagtatccacaggtgacaccacccgtcttcctgtcaccagcccttcctcagcatctacaggtcacaccacccctctacctgtcaccgacactccctcagcatccacaggtgacaccacccctcttcctgtcaccaatgcttcctcattatccacacgtcacgccacctctcttcatgtcaccagcccttcctcagcatccacaggtcacgccacctctcttcctgtcaccgacacttccgcagcatccacaggtcacgccacccctcttcctgtcaccagcacttcctcagcatccacaggtgacaccacccctcttcctgtcaccgacacttactcagcatccacaggtcaggccacccctcttcctgtcaccagcctttcctcagtatccacaggtgacaccacgcctcttcctgtcactagcccttcctcagcatccacaggtcacgccactcctcttcttgtcaccgacgcttcctcagcatccacaggtcaggccacccctcttcctgtcaccagcctttcctcagtatccacaggtgacaccacgcctcttcctgtcactagcccttcctcagcatccaccggtcatgccacctctcttcctgtcaccgacacttcctcagcatccacaggtgacaccacctctcttcctgtcaccgacacttcctcagcatacacaggtgacaccacctctcttcctgtcaccgacacttcctcatcatccacaggtgacaccacccctcttcttgtcaccgagacttcctcagtatccacaggtgacaccacccctcttcctgtcaccgacacttcctcagcatccacaggtcacgccacccctcttcctgtcaccaacacttcctcagtatccacaggtcacgccacccctcttcatgtcaccagcccttcctcagcatccacaggtcacaccacccctcttcctgtcaccgacgcttcgtcagtgtccacaggtcacgccacctctcttcctgtcaccgacgcttcctcagtgttcacaggtcatgccacctctcttcctgtcaccatcccttcctcagcatcctcaggtcacaccacccctcttcctgtcaccgacgcttcctcagtgtccacaggtcacgccacctctcttcctgtcaccgacgcttcctcagtgtccacaggtcatgccacccctcttcctgtcaccgacgcttcctcagtgtccacaggtcacgctacccctcttcctctcaccagcctttcctcagtatccacaggtgacaccacgcctcttcctgtcaccgacacttcctcagcatccacaggtcaggccacccctcttcctgtcaccagcctttcctcagtatccacaggtgacaccacccctcttcctgtcaccgacacttcctcagcatccacaggtcacgccacctctcttcctgtcaccgacacttcctcagcatccacaggtcacgccacccctcttcctgacaccgacacttcctcagcatccacaggtcacgccacccttcttcctgtcaccgacacttcctcagcatccataggtcacgccacctctcttcctgtcaccgacacttcctcaatatccacaggtcacgccacccctcttcatgtcaccagcccttcctcagcatccaccggtcacgccaccccgcttcctgtcaccgacacttcctcagcatccacaggtcacgccaaccctcttcatgtcaccagcccttcctcagcatccaccggtcacgccaccccgcttcctgtcaccgacacttcctcagcatccacaggtcacgccacccctcttcctgtcaccagcctttcctcagtatccacaggtgacaccacgcctcttcctgtcactagcccttcctcagcatccacaggtcacaccacccctcttcctgtcaccgacacttcctcagcatccacaggtcaggccaccgctcttcctgtcaccagcacttcctcagcatccacaggtgacaccacccctcttcctgtcaccgacacttcctcagcatccacaggtcaggccacccctcttcctgtcaccagcctttcctcagtatccacaggtgacaccacgcctcttcctgtcactagcccttcctcagcatccacaggtcacgccactcctcttcttgtcaccgacgcttcctcagcatccacaggtcaggccacccctcttcctgtcaccagcctttcctcagtatccacaggtgacaccacgcctcttcctgtcactagcccttcctcagcatccaccggtcatgccacctctcttcctgtcaccgacacttcctcagcatccacaggtgacaccacctctcttcctgtcaccgacacttcctcagcatacacaggtgacaccacctctcttcctgtcaccgacacttcctcatcatccacaggtgacaccacccctcttcttgtcaccgagacttcctcagtatccacaggtcacgccactcctcttcttgtcaccgacgcttcctcagcatccacaggtcacgccacccctcttcatgtcaccagcccttcctcagcatccacaggtgacaccacccctgtgcctgtcaccgacacttcctcagtatccacaggtcacgccacccctcttcctgtcaccggcctttcctcagcttccacaggtgacaccacccgtcttcctgtcaccgacatttcctcggcatccacaggtcaggccacccctcttcctgtcaccaacacttcctcagtatccacaggtgacaccatgcctcttcctgtcactagcccttcctcagcatccacaggtcacgccacccctcttcctgtcaccagcacttcctcagcatccaccggtcacgccacccctgttcctgtcaccagcacttcctcagcatctacaggtcacaccacccctcttcctgtcaccgacacttcctcagcatccacaggtgacaccacccctcttcctgtcaccagcccttcctcagcatctacaggtcacaccacccctcttcatgtcaccatcccttcctcagcatccacaggtgacaccagcactcttcctgtcaccggcgcttcctcagcatccaccggtcacgccacccctcttcctgtcaccgacacttcctcagtatccaccggtcacgccacgcctcttcctgtcaccagcctttcctcagtatccacaggtgacaccacccctcttcctgtcaccgacgcttcctcggcatccacaggtcaggccacccctcttcctgtcaccagcctttcctcagtatccacaggtgacaccacccctcttcttgtcaccgacgcttcctcagtatccacaggtcacgccacccctcttcctgtcaccgacacttcctcagcatccacaggtgacaccacccgtcttcctgtcacggacacttcctcagcatccacaggtcaggccacccctcttcctgtcaccagcctttcctcagtatccacaggtgacaccacccctcttcttgtcaccgacgcttcctcagtatccacaggtcacgccacccctcttcctgtcaccgacacttcctcagcatccacaggtgacaccacccgtcttcctgtcacggacacttcctcagcatccacaggtcaggccacccctcttcctgtcaccatcccttcctcatcatcctcaggtcacaccacccctcttcctgtcaccagcacttcctcagtatctacaggtcacgtcacccctcttcatgtcaccagcccttcctcagcatccacaggtcacgtcacccctcttcctgtcaccagcacttcctcagcatccacaggtcacgccacccctcttcttgtcaccgacgcttcctcagtgtccacaggtcacgccacgcctcttcctgtcaccgacgcttcctcagcatccacaggtgacaccacccctcttcctgtcaccgacacttcctcagcatccacaggtcaggccacccctcttcctgtcaccagcctttcctcagtatccacaggtgacaccacccctcttcctgtcaccgacgcttcctcagcatccacaggtcacgccacccctcttcctgtcaccatcccttcctcagtatccacaggtgacaccatgcctcttcctgtcactagcccttcctcagcatccacaggtcacgccacccctcttcctgttaccggcctttcctcagcttccacaggtgacaccacccctcttcctgtcaccgacacttcctcagcatccacacgtcacgccacccctcttcctgtcaccgacacttcctcagcttccacagatgacaccacccgtcttcctgtcaccgacgtttcctcggcatccacaggacatgccacccctcttcctgtcaccagcacttcctcagcatccacaggtgacaccacccctcttcctgtcaccgacacttcctcagtatccacaggtcacgccacctctcttcctgtcaccagccgttcctcagcatccacaggtcacgccaccccccttcctgtcaccgacacttcctcagtatccacaggtcacgccacccctcttcctgtcaccagcacttcctcagtatctacaggtcacgccacccctcttcctgtcaccagcccttcctcagcatccacaggtcacgccacccctgttcctgtcaccagcacttcctcagcatccacaggtgacaccacccctcttcctgtcaccaatgcttcctcattatccacaggtcacgccacccctcttcatgtcaccagcccttcctcagcatccagaggtgacaccagcactcttcctgtcaccgatgcttcctcagcatccaccggtcacgccacccctcttcctctcaccagcctttcctcagtatccacaggtgacaccacgcctcttcctgtcaccgacacttcctctgcatccacaggtcaggccacccctcttcctgtcaccagcctttcctcagtatccacaggtgacaccacgcctcttcctgtcaccatcccttcctcagcatcctcaggtcacaccacctctcttcctgtcaccgacgcttcctcagtgtccacaggtcacggcacccctcttcctgtcaccagcacttcctcagcatccacaggtgacaccacccctcttcctgtcaccgacacttcctcagcatccacaggtcacgccacccctcttcctgtcaccgacacttcctcagcatccacaggtcacgccacccctcttcctgtcaccagcctttcctcagtatccacaggtcacgccacccctcttgctgtcagcagtgctacctcagcttccacagtatcctcggactcccctctgaagatggaaacaccaggtagctgccaactgcctcgcctttatgtctcccagtgggccccttggcggaattcagcctaaggagtacctgagaacactggtgcattcgcattacctggtggggccgtgtcaggtcccacaggggaggaggtgatgggtgtggtgggtgacaggctcaccctcctttgtgccgcaatcgaaaagcactgatgtcgagagtagtttggatatgagcaggggagagacaaggagtttccagctccctcttccagctcctgatttctttgaatctctttgactctcctgttttgttactgtaagaaacaccccgccttgtcttttcacgtgtccaggaatgacaacaccgtcactgaagacagacggtgggagacgcacagccacatcaccaccccccacaacctcccagaccatcatttccaccattcccagcactgccatgcacacccgctccacagctgcccccatccccatcctgcctgagagaggtgaggccatacaggtgaggcctgtgccttttgaggggtgatgtaactgaaggctccctctcagcctacttcccacagtctccgctctctcgggtggggagggccttaccgaggacagggacacagcatcggagtcgctcctgagggctggctttgtgcatggcactgggccaggagctggagacagagaaatgaccccagtgccattcagcaagggatagatggacggtccggtagcggcggttagaggactcatcccagggtctaagtgcacacaatggaaggccctaaggaatgcagagccggggatggaggagcaccccaggcagggaggagggcgggaacagctggaacaaaggtgtggaaggtatgggtgtggaaggtatgggtgtggaaggtatggctgtggaaggtatgggtgtggaaggtatgggtgtggaaggtatgggtgtggaaggtatggatgtggaaggtatgggtgtggaaggtatgggtgtggaaggtatgggtgtggaaggtatgggtgtggaaggtatgggtgtggaaggtaagggtgtggaaggtatgggtgtggaaggtatgggtgtggaaggtatgggtgtggaaggtatggatgtggaaggtatgggtgtggaaggtatgggtgtggaaggtatgactgtggaaggtatgggtgtggaaggtatgggtgtggaaggtatgggtgtggaaggtatgggtgtggaaggtatgggtgtggaaggtatgggtgtggaaggtatgactgtggaaggtatgggtgtggaaggtatgactgtggaaggtatgggtgtggaaggtaagggtgtggaaggtatgggtgtggaaggtatgggtgtggaaggtatgggtgtggaaggtatgggtgtggaaggtaagggtgtggaaggtatgggtgtggaaggtatgggtgtggaaggtatgggtgtggaaggtaagggtgtggaaggtatgggtgtggaaggtatgggtgtggaaggtatgggtgtggaaggtatggatgtggaaggtatgggtgtggaaggtatgggtgtggaaggtatgactgtggaaggtatgggtgtggaaggtatgactgtggaaggtatgggtgtggaaggtatgggtgtggaaggtatgggtgtggaaggtatggatgtggaaggtatgggtgtggaaggtatgggtgtggaaggtatgactgtggaaggtatgggtgtggaaggtatgactgtggaaggtatgggtgtggaaggtatgggtgtggaaggtatgggtgtggaaggtatgggtgtggaaggtatggatgtggaaggtatgggtgtggaaggtatgggtgtggaaggtatgactgtggaaggtatgggtgtggaaggtatgactgtggaaggtatgggtgtggaaggtaagggtgtggaaggtatgggtgtggaaggtatgggtgtggaaggtatgggtgtggaaggtatgactgtggaaggtatggccgtggaaggtatgggtgtggaaggtatgggtgtggaaggtatgggtgtggaaggtatgggtgtggaaggtatgggtgtggaaggtatgggtgtggaaggtatgactgtggaaggtatgggtgtggaaggtaagggtgtggaaggtatgggtgtggaaggtatgggtgtggaaggtatgactgtggaaggtatgggtgtggaaggtatgggtgtggaaggtatgggtgtggaaggtatgggtgtggaaggtatgggtgcggaaggtatgggtgtggaaggtatgggtgcggaaggtatgggtgtggaaggtatgggtgtggaaggtatggatgcggaaggtatgggtgtggaaggtatgggtgcggaaggtatgggtgcggaaggtatgggtgtggaaggtatgggtgtggaaggtatgggtgtggaaggtatgggtgtggaaggtatggctgtggaaggtatgggtgtggaaggtatgggtgtggaaggtatgactgtggaaggtatgggtgtggaaggtatgggtgtggaaggtatgggtgtggaaggtatgggtgtggaaggtatgggtgtggaaggtatgggtgtggaaggtatggatgtggaaggtatgggtgtggaaggtatgggtgtggaaggtatgactgtggaaggtatgggtgtggaaggtatggctgtggaaggtatgggtgtggaaggtaagggtgtggaaggtatgggtgtggaaggtatgggtgtggaaggtaagggtgtggaaggtatgggtgtggaagttatggccgtggaaggtatggatgtggaaggtatgggtgtggaaggtatgactgtggaaggtatggatgtggaaggtatgggtgtggaaggtatgggtgtggaaggtatgactgtggaaggtatgggtgtggaaggtatgggtgtggaaggtatgggtgtggaaggtatgggtgtggaaggtatggccgtggaaggtatgggtgtggaaggtatgaccgtggaaggtatggccgtggaaggtatgggtgtggaaggtaagggtgtggaaggtatgggtgtggaaggtatgggtgtggaaggtaagggtgtggaaggtatggatgtggaaggtatgggtgtggaaggtatgggtgtggaaggtatcggtgtggaaggtatgggtgtggaaggtatgactgtggaaggtatgggtgtggaaggtatgactgtggaaggtatgggtgtggaaggtatgggtgtggaaggtatgggtgtggaaggtatgggtgtggaaggtatgactgtggaaggtatgggtgtggaaggtatgggtgtggaaggtatggctgtggaaggtatgggtgtggaaggtatgactgtggaaggtatgggtgtggaaggtatgggtgtggaaggtatgggtgtggaaggtatgggtgtggaaggtatgggtgtggaaggtacgagtgtggtaggtatggctgcagaaagtcgtcccggtgctgcatgggggtggatccccgaagcatttggggtggctgaaaatgagaagaagggtagcaaaaagtgcggccggcatgcggggaatcctgtaggcaacgggagccagggaggactcagttttgcattgtacaaatggcatttaacaagtggtgcctggagcggtccgatttgcaggcagtgaggaggccaggagagcctgcgggtttccaagcaggaccaggggagggcgccaaggagtcggcagctgcgagaaatatttgggacaaggtttctcaaactggagcccgagggcctctagggagtcctaggttaaattggaggagtcttcaagtttatctggagaaaggccgtcttaggaaacaagtctcattccctgaaaagggctttgcagttactcatcttcattgcgctggacttttgtctatttttttttttttttttgagatggagttttcgctcttgttgcccaggctggagtgcagtggcgcgatctcagctcactggaacctcagcctcccgggttcaagcaattctcctgcctcagcctcccgagtggctgggattacaggcatgcgccaccacacccggctaatttttgtattttaagtagagacagggcttctccatgttggtcaggctggtctcgaactcctgacctcgtgatcctcccgcctcagcctcccaaagtgctgggattacaggcgtgagccaccgcacccagcatagacttttgtcttttaaccctgaaaaggattgagactaagagattgagaatcgtttgctggtattctgacagcaggacctgtttttctccaagctggggaaggatgagaggcgcagtttagggagtaaaatgaccacatgcatttaatgtgggtggagagggagcgagaagcaccctagatggctgcctctggggccctcggggaacacaggacaggtgtgggcagcctgcagggagcgctctgggatccctttcagccctaaagaaggcccaggcccacttggacttcctgctcttctctgtcctggcccaggagtttccctcttcccctatggggcaggcgccggggacctggagttcgtcaggaggaccgtggacttcacctccccactcttcaagccggcgactggcttcccccttggctcctctctccgtgattccctctacgtgagtccgggctgcggcccgcgcagcctgaactcccagggcccacttctctctcctgcttcgagacggaacccagaggaagcgggaatggaagcagccttggctgggcccctcgtccatccccacagcctccttaatgtcaggcctctgcctgaggaacacagggtgccaggcgagggctgcccacctgctgggcccaccgctgcttctgcggggccttctcaggagtaaaaagctacacttgggaaactggactgttcctgccgtttccaccttctgggatttgtctctggccccctggtccctgcctcctggagcagagttggagggacagtcctggctcctgtggccctgagggaggaggctgagtccgaacacagcatgagagggcgactgagcgatggagagggtgtccacacctgctgagcgatagagagagggtgtccacacctgctgagcgatggcgagagggtgtccacacctgctgagcgatggcgagagggtgtccacacctgctgagcgatggcgagagggtgtccacacctgctgagcgatagagagagggtgtccacacctgctgagcgatggcgagagggtgtccacacctgctgagcgatagagagagggtgtccacacctgctgagcgatggagagagggtgtccacacctgctgagtgatagagagagggtgtccacacctgctgagcgatggagagagggtgtccacacctgctgagtgagagagaggtttccaccccctaagtgatggagatggggtgtctgcacccctgagtgatggagagagggtgtctacacccctgagcgatggaggggggtgcctacacctgctgagcgatagagagaggtttccaccccctaagtgatggagatggggtgtctgcacccctgagcgatggagagagggtatctacacctgctgagtgatagagagaggtttccaccccctaagtgatggagatggggtgtctgcacccctgagcgatggagagagggtgtctacacccctgagcgatggagagggggtgtctacacccctgagcgatggagagaggatgtctacaccccgaacgatagagagggtgtccacacccctgagcgatgaagggagggtgtctacacccctgagcgatggagagagggtgtccacacccctgagcgatggagagagggtgtctacacccctgagcgatggagagagggtatctacacccctgagcgatggagagagggtgtctacacccctgagcgatgaagggagggtgtctatagccctgagcacccctgagcgatgaagggagggtgtctatacccctgagcgatggagagagggtgtctacacccctgagcgatggagagggggtgtctacacccctgagcgatggagagggggtgtctacacccctgagcgatggagagaggatgtctacacccctgagcgatgaagggagggtgtctacacccctgagggatggagagagggtgtccacacctgctgagtgatagagagaggtttccaccccctaagtgatggagatggggtgtctgcacccctgagcgatggagagagggtgtctacacccctgagcggtggagagagggtgtctacacccctgagcgatgaagggagggtgtctacacccctgagcgatgaagggagggtgtctacacccctgagcgatggagagagggtgtctacacccctgagcgatggagggagggtgtctacacccctgagcgatgaagggagggtgtctacacccctgagcgatgaagggagggtgtctacacccctgagcgatggagagagggtgtctacacctgctgagcaatagatagagggttccaccccctaagtgatggagatggggtgtctgcacccctgagcgatggagagagggtgtctacacccctgagcgacggagggagggctccacccactgagcgatggagggagggctccaccccctgagcgatggagagagggttccactccctgagccccacctaatgtgtcgcaaacttcaggatggggcccaggggactggccaagaggggtgtgcctgtgaggggctggtccacagccagggatctgcagtgaaacaggaccaagcctaccacccgaggacgcaagggatggcctgagggcggagcattctgtgagaatatatggaaagggctctccccaaaacaggacacaggaggtgacttccaggggttgttagcatctctaaagctctacccttgggggtcacgcttttgaggagctggaccaaggctgggactttcctccccacttccctccctgaatgctgaccacaaaacccatgtgctcagttcacagacaatggccagatcatcttcccagagtcagactaccagattttctcctaccccaacccactcccaacaggcttcacaggccgggaccctgtggccctggtggctccgttctgggacgatgctgacttctccactggtcgggggaccacattttatcaggtgagcctttcaaagcctggcagtcaggatcccccagcagctggcaggggagacaaagagctgtgtggaaggctttgccagagttgctgctgtgacagcccctgcagcaggggactgaggcttaaatatgggtgtgggaggaagcagtcaagggacattaagctgactcaggagtaccccaacccaaccacgagactgaccaggaataccccaacccaaccacgagactgaccaggaataccccaacccggccgcgagactgaccaggaataccccaacccaaccacgagactgaccaggaataccccaacccaaccacgagactgaccaggaataccccaacccaaccacgagactgaccaggaataccccaacccaaccacgagactgaccaggaataccccaacccaaccacgagactgaccaggaatacaccaacccaaccacgagactgaccaggaataccccaacccaaccacgagactgaccaggaataccccaacccggccgcgagactgaccaggaataccccaacccggccacgagactgaccaggaataccccaacccggccacgagactgaccaggaataccccaacccaaccacgagactgaccaggaataccccaacccaaccacgagactgaccaggaataccccaacccggccgcgagactgaccaggaataccccaacccggccacgagaagacctacaggtgacgggggatgggggctgacaattcagcctttgattgtaaaatcaggctgtagggggtctatcctaacaagagtgagtcaagagggcccgtcctgtgctcctggactgggtgaataactccaatcagaggaaatgtgatgaaagctactgcaggaagtgtggaccgtgcccctgccacggacagggcagtgtggaggctccaagaggaagagactcacgcctgttctcaagaagctgcagattaggcatggggaggtaaaagtgggcccgagtggctctgctctctgggccagtttgagctaagatgcttgagttcaagtccagaggtcttggacgtctgggccacgaccctgcattctgcacactgcaggccttcctgcagcccaagtttgaattgaacttcacctcagaaaagctcacggcaaaaatgcaggcctgggacaggggagggcaggccattgggtgggctgaagtgggcagggtcctcaggcagaaagtggggggcccatgtgtaggagtgggaccctcgttggcttgttttcatctgccctcctttcctaggaatacgagacgttctatggtgaacacagcctgctagtccagcaggccgagtcttggattagaaagatgacaaacaacgggggctacaaggccaggtgggccctaaaggtcacgtgggtcaatgcccacgcctatcctgcccagtggaccctcggggtgagtagacccctgggcagctcccaggagctatctgggagtcagacatcctagaatcctaggcaggggccactcttccaaaatcctctctggctctaggaaagattgctaattatagcaagcccccccaccgccacccccccactgccaccactgttggctgagcactcaccgtgggcccggcctgtctcggccctttgcctacttgacctcacagcagcccagcaaaccgggtattattaggctgaaacatagcaagtggcatttttgtaagtcaaaaatgtctggctattcacaatttcacgtggctcagtctggtattatgctccccattttacagatgaggaagacaatggttagagaggttaagtaactcgttcagagtcacgccactggcaaatggcaaagccaggcctgagtggagcttcttctgactccagcctctggctctgaagcacaggctcatccaacgggcacagggtgcgccacaccatctcctctgtcctccctccgaggaccccgctcttgtagtcaccaagttcttccttatgcctggtctaaagtcactcatttcttcattcactcgacaaaagttcactggtcacctactgtggacaacgtgtgcgccttcagtgaaccccagtgtcatccatgctgtttagatggaggagacaatttgggcaatgtccttaaatatccgcgcgggagctaatgaagcctcctctctagctctctggtatcttggctgatctcaggttctggtagctttctctaacaaacactcatctaactgaattgtaacccagaaccctttatctcccctcctggaagaaagtcaccaaaaggcaaatccaccgtggtaagtgcgttcgcacccagggtgaaagctgccctcactgacccaaaacttgccagcattttgggcccatgtctttgctgtgggcagaccaggaacaggtgctgtgcgtgtcagagcagggctgtgggacgggcggggggacataagagaggagaccccggatctctcacgggcatccctgtgttttcttccacccacccccagagcaacacctaccaagccatcctctccacggacgggagcaggtcctatgccctgtttctctaccagagcggtgggatgcagtgggacgtggcccagcgctcaggcaacccggtgctcatgggcttctctaggtaggatgggaggggctgtcagcactgagcagttggcagggaggggtgtagagtcggctttcgctgcacacacactcccatcctggggcaaggcgggaaccctcctggctggtgcttctgactcacgctgactccagctcccgctggggccaggcatctggctgcttcccacgacaagatcacccatttgtccgccatgccctttcccatcccagtccccgtgagttgtaatcattcccagagctttggcttccctgatttccactgacattagctggcctctcttaccttctcaaatatcgtgataataacagccaagactgatgacactaacacttcgtgtacattactgcatgtcatgatcacagccaccccacgaggcagatactatttatcatccccattctacagatcaggaaactgagatacaaatagactagcaatttgcctaaagtcccagagctgggaagtggcaacatggagatttgaacccagacatctgaggctagaatctgccttcctgacttcctgagaattacccacttatccagggggcacccaaaagcttatatggagccggtggtgaagggagagaggaggcacggggctgtgggcggttgaaatgtggattcccgtgtgtgggaggctgtctccgatgtagcagtgcgccatctctgttcccatatgctggggtgtggcaatcagtacgaaggccacatgtcttttgttcttgataattgctgcaaatgtcttctcttacattgagtgtgttcaggttacccctttgattacactgctttcactagtttcttggtgtgctatgaaggagaaaggaggggaattacccaactctaggaatttcacacggtccaggtacacccaaggaaggtgtcacgcaggggtgcatcatcctgcttgtgtcagggaggaggaaaagcccaagagtccccactctgcagcaggcacccagggtggggctgcaggcggctgctccaggctttcctccttcccgggccatgctcggaccccacacctggggacggtgatggcgatagtggtggtggtggtggtggtggtggtggtagtggcggtggaggtggcagtggcgatggtgatggtggtgatggtggtggtgatggtggtgatggtggtggtgttgggggtgatggtggcgatggtggtggtggtgatggtggtggtggtggtgatggtggtgatgatggtggtggcaatgatggtggtgatggtggtggtggtggtgatggtggtggtggcgatggtgatggtggtggtgatggtggtggtggccgatggtggtattttttggggggtggccagtgatggtggtgatggtgatggtggtggtggtggtggtggcgatggtggtggtgatggtggtgatggtggtggcaatggtgatgatggtggtggtggtgatggtggtggtgatggtggtggcaatggtaatggtgatggtgatgatggtggtggcgatggtggtggtgatggtgatggtggtggtgatggtgatggtggtggtgatggtgatggtggtggtagtgttggtggtggtggtggtgatggtgatggtgatggtagtgttggtggtgacgatgatggtggtggtggtggtagtgctggcgatggtggtggtggtggtagcagcagtggcagcatctgttgtatcaaggcctcactcagttctgagggatttgcagatgttactgcatttaatcctcacaatacagtgaggtagatgccactgtaatcccttttatacagatgtgaaaactaagaatcagagagggtcagtaacttgtctgtggtcaccaagctagtaagtggggggatcagcagttgaatccatcactgcctaactccagaaactgggtcttaaccatcacaatacactgtccctagagtctgtcccgctccaaaccctctgcttgttgaagggaaacaaacagaaatgaggcctgaactctgttgttttcacgcccctgtctcatgaccaagcggacctgccagtttcttgcagctagcgatcagatgtggaatcggttagagcctcctacctctgcccagccatcttgtcccctctcctgctcagtgctggggcttgacgatgggggctggctgacccactgacccactgcccgcctagctggttgaccagctgctgaccggctagcgggatgaatggacgccttgcaaccgcgaggcaagagcctgtgcacggcagaggcctgagagtctctcctttcctgcagtggagatggctatttcgaaaacagcccactgatgtcccagccagtgtgggagaggtatcgccctgatagattcctgaattccaactcaggtaaaagtgccaccttatcacacctgagctggtctcaagccctcgcgtgtcctccagcccatacaccatcgcagtcctagagggcacctccctaacatcacggccatcctgaagggcctcccccacacagttcaacctcctacaggtctaggtgggatgtggcaccacccagggagcagctggcacctcctgcacccctggacacagcacagtctcagagggggctacatctcctccctcagatgaaccagcccaagagggctctcggggggcccagatcctggaagtgaggacagggtcttgtgggaggtgggatttgaatgggcagttcagacaatactccaagtgtcctgtccagagcaggaatgagaacccaggtgagatcctttaagaaaatctaccggctgggcgccatggctcacacctgtaatcccagcattttgggaggctgaggtgggcggatcacttgagatcaggagttcgagaccagcctggccaacatggtgaaattctgtctctactaaaaatacaaaaattagccagacgttgtggtgcacacctgtaattccagctactcaataggttgaggcaggagaattgtttgaacctgggaggtggaggttgcagcgagccgagatcgcgccactgcactccagcctgggtgacagagtgagactccatctcaaaaagagagaatccaccaagaagaggcccgggggtctcacgttcattgtcatcatttactgggcacctgccgtgtgctggacaccgtgctaacagccagaaacaaaggaaaaagaccaactgtgttttctctcaaagagctttcatgcgggtagaaagacaagcgtcaagaggcaaacaatggcgccttctccatgactcagtccagaggccccaatgggaaagaaataatagtttccaaaatgtgataggttgaaggacattttagccttagaaaccagccacgtgaactgtgaagtgtaaatccagttctatttgttctgtggcaaccacgtttggtcgtcagaagcagacgctaagtcaagcgaggggctttgggggaggaattgttgcccatctgacttgggtccaaagaggtgagaagtggcaggagggggtgcatttgtgagttttggttgaccctaaagctgagacagggcctgcagccccagccctggataagccctttcccccccatccccgacctcaggcctccaagggctgcagttctacaggctacaccgggaagaaaggcccaactaccgtctcgagtgcctgcagtggctgaagagccagcctcggtggcccagctggggctggaaccaggtctcctgcccttgttcctggcagcagggacgacgggacttacgattccaacccgtcagcataggtgacacctccttcccgccccccacaagcccacccaccaccctctctgctcacgccctcagcctctccccagaaacagcccctgcttgttcccaccccgcccctggcagccccagcctgggcctgagtgggactggacttgtttcaggtcgctggggcctcggcagtaggcagctgtgcagcttcacctcttggcgaggaggcgtgtgctgcagctacgggccctggggagagtttcgtgaaggctggcacgtgcagcgtccttggcagttgggtgcgtgagtccgtgatctcaaccccaccttcccggccaagtaggggaccctcagcatgaagcctctcgtcctcattccttcccagacccttcccctctctgggcctccacttcctgatcgggtaacgttaggaagcttccaacatccccaccagcggacattcggggatgtatgagctgagagcctctttcctcatctctatacctggctccacatccccaggccagagtggggccatttctccaggcaagaagagagcgcctaggctgaccccgtccctgtagacccagatgagcaggatgtttgggggaagactgagagtcagctcccacagagacgctccaagggagctggagagcgcaggggtgtggggtcacgtggtctgtgtcctcccgttctctcccccgtcccatggcagtttccacagttcccagaccgtcagccgtgccctccccactcccagcacgttgagagcagcagtacttggctctccaccctcaagacaggaaatttgcttagctcctaccatgcgccctgctgcacagtggccccaatgcctgcaaaagagacagcaccctattcactgcccaaggtcatgagaagggccatgctggattccaaagccatgctcttgccaccaacaccgccctgccctaccaagctctccaccaacaccgccctgccctgccaagctctccaccaacaccgccctgccctgccaagctctccaccaacaccgccctgccctgccaagctctcaaccaacaccaccctgccctgccaagcttctccaggtcctcaacctccccgactcactgctgttctccgcagcccaggaactggagccacagagctggtgctgccgctggaatgacaagccctacctctgtgccctgtaccagcagaggcggccccacgtgggctgtgctacatacaggcccccacagcccggtgagcgacagggcccaggcccaggaagagcctctggggagggggagcttctgggcttccgggaggtggcatctggataaggagtaggggcagagctgtggccacaagggaagatggagatgacgccagtgatgggtggatggtcagtggaggggctttgtacctgagttggggaaggagtgagaatagcggggcacgaaaatgcaaggaggcttgaggggaggattctgtgctctgtgaacatctccccagctgagtccgcccggacaagtgccccagtcgactgcagtgaggaacctcaccctgagtcctcctggacaagtgccgcacctgactgcagtgaggaacctcagccctacacatgatttgtcatgctgaggcagcatctcttttttccttccagcctggatgttcggggacccccacatcaccaccttggatggtgtcagttacaccttcaatgggctgggggacttcctgctggtcggggcccaagacgggaactcctccttcctgcttcagggccgcaccgcccagactggctcagcccaggccaccaacttcatcgcctttgcggctcagtaccgctccagcagcctgggccccgtcacggtgagtgaggggtgccgggaacctccctgcattccacccacagggaccttcagccacatactgaggccgaggagaaggaagagagcgaaggaggggaagccgggcaggagggagggaggacacagcccatccacgcagctgtcccggagtaaatcctggggatggtggattagggctctggccccacggtttcccagctgcttggcttcgaagaaactacttaatttggttttctcctagtaagaatggggatgacattacctgcctcacgggctgttgtaagagctaaagggcataatccgaaaaccatgcctggccctgccgagcacacggtagacggcggctggcatcacctcaggccgcggcctccagcgccttcctcccggcccagggcgcagcttccagccccaggggctctcccagctgctttcctgggcgtcggctccacctgcgggtcgggctcaggccccctcccatctccttccaggtccaatggctccttgagcctcacgacgcaatccgtgtcctgctggataaccagactgtgacatttcagcctgaccatgaagacggcggaggtaggttggggagcgccggccgccccctccccgcaccgggagcagcgaggtgggcgggaagccgcgttgcggtgcagggccgggcgcgtggcggtgcagggccgggtgcgttgtggtgcagggccgggcgcgtggcggtgccgcgccgagtgctttgcggtgcagggccgggtgcgttgtggtgcagggcccggtgcgtggcggtgcagggcccggtgcgtggcggtgcagggccgggtgcgtggcggtgcagggccgggtgcgtggcggtgcagggcccggtgcgttgcggtgcagggccgggtgcgttgcggtgcagggcccggtgcgtggcggtgcagggccgagtgcgttgtggtgccgggcccggtgcgtggcggtgcagggcccggtgcgtggcggtgcagggccaagagagcgcagcctctacccccgagcggggcgtgcagctcgccggcctcttctccgcctccagtgcagacccctcccggcttcagccccaggggcggggtgggggtggtgcgggcccggcagggcgcgggtttggtgcgggccgtggtgccgacctggcttcctctccgctgcctcccgatgctccaggccaggagacgttcaacgccaccggagtcctcctgagccgcaacggctctgaggtctcggccagcttcgacggctgggccaccgtctcggtgatcgcgctctccaacatcctccacgcctccgccagcctcccgcccgagtaccagaaccgcacggaggggctcctgggtgagggcggctcggacctgcctctgaggctccgcggagccagccggagctcggacccccacgccggcggcccgggcagccctgctcggcctccctttctccgcctccttggagcagaacccttggggcacagagcgggccgggagccgaggggcttctccagcctcccccgaggctcccttcctgtcctccgcccgctctaagggagcatcagggggggctgcggggaggcggggggcacagccatcctttctccctttcctcttgcctcccacatcctcccgccctcctccaccgctgcccgcgtttcctcccgcccctcccgaaggcagacgggcagggtgtgagggcccgtcctcccggctcccctggaggcctgacagcaggtgcaagagcagaggctgccaggccctggcctctccccactgcgtccgccggatgctccccaggaaggggacagccgcgtgcccagggtggccaacctcccactctgtccctcaggggtctggaataacaatccagaggacgacttcaggatgcccaatggctccaccattcccccagggagccctgaggagatgcttttccactttggaatgacctgtgagtctgggcagggtcctggggcagaggggcaggtgaggggagccggtatgtttatgtcgtccccctcggccctgtaggaagcagacctccatcctccctaaggtctgaggaatctgtgcccccccagggctgtccccaccaccaccagcccacctgcctctctctacctggaggagagaatgggggacgtggagtgtcctctctcactgcagcaggtgtttctagactcagaagctggaaaccgctctggccctgcactcccaccgccccgctccaagccatctaaagtgaggggtagaggtggacacagatggaataaggctgagtgccatgcctggtaccaccgcgctctgtgtgttacagggcagatcaacgggacaggcctccttggcaagaggaatgaccagctgccttccaacttcacccctgttttctactcacaactgcaaaaaaacagctcctgggctgaacatttgatctccaactgtgacggagatagctcatgcatctatgacaccctggccctgcgcaacgcaagcatcggacttcacacgagggaagtcagtaaaaactacgagcaggcgaacgccaccctcagtaagtggcccgaggcctggggaggccttttcagagtcgggagcagatgaggagctgcccttgcctgaccctgcttttccctgtgcatctgcattcactgagcagattcttccactcctggcattcctctgctcaaacccttcagagacttccctggctctctccatccttgcagtggccttcggcccagttcagcttctcagagctcttctcctagtgctggacgccttcccagccccctccgtccatcctagcgctggatgccttcccagcccccttcactccatactagcgctggacgccttcccagccccctccactccatcctagcgctggaccccttcccagccccctccatccatcctagcgctggatgccttcccagccccctccactccatcctagcgctggaccccttcccagctccctgcactccagccccgcaggctcctctgtgttcttcaaacacgctaggtgcgctcagctcccaggcttcacacgtgctgttctcttgcctggaatacccttccttccctggacagccacacgcttgcccctcaccttctttacgtcttcattccaatgtcccctccttggtgaggcctctcttggccgccctgtctaaaatgtcacattcacccacacttcatgtttgccttccctgctttatttttttctccttagcatttataattactcaacatattttataattttcacaggtatctttttaattattcattcatgactgtatcctcaccacccagaacagtgccagccacttagctcaataaatgtttgttaaatgactgactgaatgaatgtgtgaagcaactatgaaatggaaatggcagggctccgagaaacagacccgtgaagaggtttcactccctctctatttctggacaaatgcaatgtccctttagatgtgaccctccaggttttgtgcgtgtgtgtgtgtgtgttgacggagtttcgctcttgttgcccaggctggagtgcagtgacgtgatctcggctcgtcgcaacctctgcttcccgggttcaagcgattcttctgtttcagcctcctgaatagctgagattacaggcacccgccaccatgcctgactaattttgtatttttagtagagacagggtttcaccatgttggtcaggctggtcgcgaactcctgacctcaggttatccaccggcctcggcctcccaaagtgctgggattacagacatgagacacccagcacccttcaggttttcagccctttgccaaaggtacaaccctttggtctaagggtacaaccctttgtaatggtccaaacaaatctgctctctaattcactttttgtcatacccagcatagcactgtgttcaggaaagaatctgaaaggagctctttttgaggaggtggggggagagaaagaggctaaagatttggctaggtgggcagtacagtcccagcctgctgtgcgtcagtcgagagcaggtacacagaataattccttcttccagaggtgggtcacagtcctgggaatgcctcacacatattagagttgagaaagagaggaggctgataaagcaggagactgtcctgcctcggaacccccattccctctttgtgtttcagatcagtacccgccctccatcaatggtggtcgtgtgattgaagcctacaaggggcagaccacgctgattcagtacaccagcaatgctgaggatgccaacttcacgctcagagacagctgcaccgacttggagctctttggtaggactatttggctggctggggagagtggggaggtgggtaggggatgaggtcagagtcaaatttgggaaattctgcattgctacacccagcaacacttgctgtcactctttcatttgaatatccaggaccggcatttccgaggagcgatatctataactcaaataggtgccccccagtaagtagtagtgacgggcttacggcggtttggccagatagctagagtgactctgggcacatagaactgattcaaggctgggcgcggtggctcatgcctgtcatcccagcgctttgggaggccgaggtgggcggatcatctgaggtcaggagttcgagaccagtctggccaacatggtgaaaccccgtctgtactaaaaatacaaaaaaaaaaaaaaaagccaggcatggtggtgcatgcctgtaatcccagctactcaggagactgaggcaggagaatcgcttgaacccaggaggcagatgttgcagtgagctgagatcgcgtcactgcactccagcctggtgacagagcgagactctgtctcaaaaaaaagatttgaagatcatttaatctcaatctgatcatatatagtcatctttagtcattatgcattcaccaaattattagaataatcaagtgcaacatttaaaatgaccaatttacaacatttttatatctattaaaggaaggctctaatgtaggacaagatttaaagaaatatgaaaaatactttcgtttggtacctttcttgctctttttgtctggcaaccatgtttacctagccgtgtctctccttgtctggtttcctgggctaggacccagtagagtgccatcgctccctctccgtaaaggtgttagagaccccgagccctacatccatgatcagggccaaaggctccaagcacaagggacgctgctttcgtctgttgccagcactaattcaaagggtccaagggcatcaaagggatgccactttctgtctgttgccagcactaattctgagggtgactgttcttggctaaaccaaagccccggggggcatattttgcgttctctttgcctctctgcctgatgttctttctctctgtgtctcagaagggactgctttgcctgcatcccttttgccccatgtgcgtcctcccaggtgcgagacaatggcactgagaggttttcttctccacgggccccacccctgaaggacgatggtctggaaattacgttcgtctattctctgtcacttcctttcaatctgctgttttaaggagactttcctattttcttgaaaaatatcagtttgatctgaagatatgtagtagtgtttcactacagtctggtgtctgaagggggtgtctgggaaaggtccaggaagtggggggtgggtgagaaatcagggcaaaggatggggctgccgtgagagtgaagccagtttggccggcagcgcagcctggggaggtgtctggaggatcctggccttgatcctccatcccccaggatgtcccatcctggtgtgagcccagccagggctgccctttggggtttcttcaggaggaaactgctttcccctctgtggcgttccccgctacaatcagtatgttggactggtgccacgtccttccagctgggagcgtgaccaggatcacccccgataccagccttgctcaaaggaaatgaccaatgagatttgtctggggagggggagggtggcctgagaggggtgggggaagccccgtatcagcaatcagaccacagagccgaggagtctcccagctctcaacattctcatcttccccggggcagagaatgggacgttgctgtggacacccaagtcgctggagccattcactctggagattctagcaagaagtgccaagattggcttggcatctgcactccagcccaggactgtggtctgccattgcaatgcagagagccagtgtttgtacaatcagaccagcagggtgggcaactcctccctggaggtgagtgttgggaggtgggggaggagtttctgtgccgaggggagaggaaatgggagtggaatggatgctgtgattctgcctgtcctgggtgtgtctgtgttgggagatgggtggagcagtggtaggtgagtagagcagattccagtctcaggccacaaactcacatgggaagaaagagaactttctggccgggtgcagtggctcacgcctttaatctcagcacttttggaggctgaggcaagcggatcacaaggtcaggagatcgagaccatcctgaccaacatggtgaaaccccatctctactaaaaatacaaaaattagctgggcgtggtggcaggcgcctgtagtcccagctactcgggaggctgcggcaggaaaatcgcttgcacccgggaggtggaggttacagtgagccaagattgcgccattgtactccagcctgggcaacagagtgagactctatgtcaaaaagaaagaaaagaaaagaaaagaaaagaaaagaaagaaagaaggagggaaagaaaggaaaggaaagaaaggaaaggaaagaaagggaaaggagaaaaagaaagaaagaaagaaactatattggagaaaaagaaggacgaaagaaagaaaaaggaaggaaggaaggaaagaggaaagaaccttactttattgcttatagttcaactggatttttatctcccacctccccttctgccaggttggcaggaaacttccacctccgtctttctcacagcgccccacagctctgcggcgaagcccagcagagggccctgcggtgtggggtggaaggtggttgtgggtccctgggcgtgagtccacacaggtttccatcacagctccgccccacttccccttcagacccagggagaggctctgctgcttctgtgccttgctcagacacaggacaccttttctgaggccgcttacagtcctgtctgcctagaagcaccccacagtcatttctccctaggatgtcatcatcacggtgctgggaagagagctgcgggtccctctatcttgacacctccaagcccctttgctttctcttttactatctccttccagctaaaagaaacatcttttccagtttggagaaactacttctccatgtttctagccaagatacttggcctaattccacgactcttgtcctatctgcttttttatttttatttttggttgtaaaggaagagccagaaaggaaagtgtttatctggcaacacaaaactacctccttcaccacttacacacacacgcatgcacacgcacacacagacacacacacacatccttctgaagcatgagcagagaatgggtactcgaaagggatagaggtagggacggttggtggggtagggggtagaaaagcataaaatacacaatgggaaaaagagattgaaatcaatatactattccacagatccccaaatatccactcttgggagtagcaagtgttagaggattccattttagggagattccccgagtcgcttcagtaagggagttagaaaggggagcgcagggagcttggtgaggtctcggcgccgcagcctttgctgagctgctgtactggctgctgtgttttctcacagtgctcagcagggaggctctgtcttcttggtttgggagtcagcaagggaggtcaatttcagcttatatgaaatccagtttgcaggccacataggagcctgaggcaggcggattgcttgagctcaggactttgagaccagcctgggcaatatggcaagaccctgtctctactaaaaatcaaaaaattagctgggcgtggtggcatatgcctgtaatctcaagtatttgggaggctgaggcacaagaattgctttaacttgggagggggacattgcagtgagccgagatcgagacactgcactgcagcgtgggaaacagaacgagactgtctcattaaaaaaaaaagcagaagaaaaatacaaaaattagtgggcgtggtggtgcatgcctgtagccccagctcctcgggaggctgaggtgggaggatggcttgaacctgggaggtggagtttgcagtgagccaagatcaagacattgcactccagcctgggcaaccgagtgggaccctgtctcaaaataaataaggaatccagttttcaagcaataggacatacatggatacatacacacatggatacatacacacatggatacacacatatggatacatacatggatacatgcatacatgcatgacacacacatacatacatggatacatacatggatacatacattgatacatatctacatggatacatacatgatacatacacacatggatacacacatggatacatacatgcatacatgcatggatacacacatacatacatggatacatacatacatggatacatacatctatacatggatacattcattcattcattcatgcttcaatcattcatgcatcccccatcttactctagaaaggatttcaggcaaagagctagagtgtgagagggaagaggctgagatcctggtgcagggccaggcagggggtcagggtaaacaatgacccagggaggccagctgggcaggactaccgtgtggctttaggcagggccttgccgccccacggcctggtcaagaaggtgctcagcaggtgctggtggggctgagacatgactcagggtccacgggttctcaggccaaaggggtcagtcagaaacacccagaagcccttcccagtttggtctcctggccgcccgtgatcggcaaccctcctccaggtggctggctgcaagtgtgacgggggcaccttcggccgctactgcgagggctccgaggatgcctgtgaggagccgtgcttcccgagtgtccactgcgttcctgggaagggctgcgaggcctgccctccaaacctgactggggatgggcggcactgtgcgggtgagccgggaacagggcctggagcaggcgcttctgggagcagctgatagctcaagggtgtagacagccaaaggcaaaccatttctctcctttttccagcagatctttagaatgctcaatctaggcaggtgtgggaaatctaggcaggtgtgggaaatcatctaggcaggtatgggaaatctaggcaggcctggaaagggggtggtggatggtgtggggctgaaggagaagaggttgtacaggcataggggaggggacggggctgggcatccctgaggcattgatggggggagccccgggcgaaagactgaagatggtttggggaggagacttcagcagccgccagagaaccgggcaagctgggtcctcgggatccctggggatcttcagagacacgaggctcaggatctctgcatctcacgagtcaggacgtttggaggggctggcgtggggatccggcagcaaaggtgcctgattttccctttgagtcctcccagccatctgttcctcccgctctgagtcaccagagtctgatgagggagatcacagcgaggcctttaccaagccccctaaggcacccaaataaaatctacagatcactgtgccttcagagccgaggccagagtgggtagaaccgcagcttttataaaggcagaaaaggagatggacgtgagaggggagggcgtgaggaaagccagctgggggcccctcctctgccagccttcggggtcctttctgaagcagagggtctgagaaacactccagtccctccacaaaagtggaagaatacctgtctggccggggaaaggggttgccctgcagagaaacgcctggaaaaggggggaaggatggtttggggttccgagtggcccgaagtggaacactgggggaaagcaagcctgtcccggagcgggtttccacgggctgggccgtccctctgctgccctgcgcgctgctgctgacctccctactcactctgcagctctggggagctctttcctgtgtcagaaccagtcctgccctgtgaattactgctacaatcaaggccactgctacatctcccagactctgggctgtcagcccatgtgcacctgccccccagccttcactgacagccgctgcttcctggctgggaacaacttcagtccaactgtcaacctaggtaccgccagagaccccgccctctcacccccgcactcttcctgggccccaccctctcacccccgcactccgcccacctttggggaagatgaggaagctctggggtcacaggacagagctccagatttctctgggatggtgtaaggtgcgggctatgggagctggcgaggcaagctgtcacggcaaggaccacgggcctgtgtggcctatggaggaaggacggaggcagagacctcacagctggctcagtgagatgagcgctggggaggcccggagcatagtggagtgagccctagcgtgagggccacttctcccggtttcttcagcaaccttctgtcactgtggaatgtagggtgagggccacttctcccggtttgctcagcgaccttctgtcactgtggaatgtagggtgagggccacttctcccggtttcttcagcgaccttctgtcactgtggaatgtagggtgagggccacttctcccggtttcttcagtgaccttctgtcactggaatggagcagtcaacttgggctggcccgacagactttttgggtaagtctgggtaaaccgtggggtgatgatacatttgcttctcccatctccagaacttcccttaagagtcatccagctcttgctcagtgaagaggaaaatgcctccatggcagaagtcaacgcctcggtcagtgctgcaggccgcgctctgggtgggagggggcgcttggcgggttcaggccagggcggaaccatcgctgtgcggccttcatcttgtcatccatctggattcaactgccagaggaggccggagcctcttgccccatgggaggtgcagggcattaggaagtgaggaaggcccaagacagaaacctcgactcatcataagcagaggccagggtgccaagtcaccccagccgagacctctaagcatcttggttatgatctgaaagaaactaaaggacatttcacctcccgggagtcttccctgacttcccagagggaacgggcggctccctcttcttggctgccatgccatacctcagtcacaggcaaagtggcacaactgctgagtggttggggctgtagaatcctgcacacccagaatcagaatccctgctctgcccctcactagagcaggtgtgctaactagacacatcatataacaggtcatctgtaaacacagggacaatcatagcacctgagtcaacaggccgtatcaccgtttaaatgataacgcgtgtcaagcatttagtccagtgcctgacacacaagtattcaaacatgatgactgctattatcactactgcaagcctagcactccccactccacactccacacggtaaaatctgttgacatttgtcacctgcgccagacaatgcactctttgagtttagactccacctaagtcaccttgacacccccagccccgaccatctcctaggtctaaacaacccatgttcaatatactggggaggaggtcatgagtcatgtgtcagaggcaaggtaggggccattcatgtcagattctctgctcgtcatatgaggctgagggggggacagaatggaaaacccttcactctcaacaaacattattaagcaaggacccatgacactcactgagctaggctagagtgcaagggtgcagagacaggtgaggaaggtgctggggtcctggggctcccgttccaggaggaaacagggatgaatacacccatcaaggtgggagggcgtctcccccccggatggggcctcacccccacccccatctgccatcctctaacctaggtggcatacagactggggaccctggacatgcgggcctttctccgcaacagccaagtggaacgaatgtaagtgggactgtgtccccctaagcccccagatctcttcctcatcccccacccccagccccccaccccccctcaccgttgccctcccacacagcgattctgcagcaccggcctcgggaagccccatccaacactggatggtcatctcggagttccagtaccgccctcggggcccggtcattgacttcctgaacaaccagctgctggccgcggtggtggaggcgttcttataccacgttccacggaggagtgaggagcccaggaacgacgtggtcttccagcccatctccggggaagacgtgcgcgatgtgacagcccgtgagtccgtccattccggggacactatgggggtcactgcggggggcgggcaaacagaggtgcttcagcccacacaaacaagctaattgagtttttctgtttgtttggttttgagcgaaaacgtgaacattttgtccagctgctttttagattcgagagcaggagcagccagcactgctgaggcagtcacacgtatacagcttcacggagcaagcacccagccagggccttgctgactgtggctgctaataaaacagcagcaatttagtttcataaaattgctaatagttttcttaaaatgccattgcacttaagcatacacagggacacccccatcgtctctgctctccagcgggaggatatgaagctggagactggagactggccagggacagaggcaactactccctgaaatggtgtcaccctgtgcaagcaccttccccaggccaggccaggcctcaacaccccccagcaccttccccaggccaggccaggcctcaacaccccccagcaccttccccaggccaggcctcaacaccccccagcacctccccgaggccaggcctcaacactccccaagcaccttccccaggccaggcctcagcacccccatctgaaaatgagatgggatttctgagccccctttcagtgctgacagcccccggttttcctggaacaggaataagttggcaagcttgtcagagaaacaaaggaataacacgtcctattttctgtatagggcagagaacagggcaggggccgccccaaacacaacaggaactgctgttttacttgggtctgggaccaagcaagagcttctcccagaatccaggctaagcccgctctctcccggagcagggggtgaaatccctcagaactactgtatgttggggagttggggtaggaggcgcagggaggcaagccctgactatgcaattaaggaaaacctataatttttattatacaagcatcgtatgtttattgcagaaactttaggaaacacaaacttaacaaaaagaaaagaggaaaaaaaccctgtaatcccaatccccagagagacaacagtatttcctacatatccttctgaactttcttttatgcctaggaaccttcagacatccattttttactgaaggatcaaatcattcctaatgttggaaaacctcctcattaaacagttgtgaacaggttgtctcatatattctgtccacattcccatttgacatattatgatggtatgtttgaatatagtgtcatattttaaaatacattataataatcattaccattgtttgagtgcttactgtgtgtccggcactgtactgagtactgtggacacattatctcatttaaatctcacagcagcataagtgctattatgatcatttccttttaaagataaggaaactgaagctgaaagagtgaaggaatgttccctgaattacacatctagtcactagcagagtttcagtttccacccaagtctggccaccttcaaagcatgtgctcttcaggaaagcattttccataggaacctttttccacaggaacctttttccctaggaacctttttccctaggaaactttttccataggaagcaccatcccttggtaaggatgtgcagtagtttatttaaccagaccctacttctgaaacgttactattatccaccatgctgatctgaacgtccaggggcatatgtatctgtgcacttctccaattatttctcaggataaacacccgaaaaggaattcgtggctgaaaaggtactgatttagtgttcatcggttgctttctgtgttaatctgtgtccttcccgacagtgaacgtgagcacgctgaaggcttacttcagatgcgatggctacaagggctacgacctggtctacagcccccagagcggcttcacctgcgtgtccccgtgcagtaggggctactgtgaccatggaggccagtgccagcacctgcccagtgggccccgctgcaggtgcatagggctgtggccaggaggtggaggacagtgctggggaacccaagctgggcaagacactgcaaggggtccaggaattaggatggctcgagagatcagagaccagggaagagaggacagtggaaaaggagagttgtgagtgcctgctctgtgtggagaataaaggcgctatattacaaactctgaaacccaaaagagccataggggggcttagaactatttcgaagatgagaaaactgcagcttagagatgacaaggaacctgccccaggacgcagtaggttaagcagcagggcatggtttccaaagccaaacattttttatatttttatgttttgagatggagtgtcatttgtcgcccaggctggagtgcagtggtgcgttctcggctcactgcaacgtccttctcccgcgttcaagcgattctcctgcctcagcctcccgagtagttgggactacaggcatgcaccaccacgcccagctgatttttgcatttttagtagagacggggtttcgccatgttggtcaggctggtctcgaactcctgacctcaggtgatccgtccaccttggcctcccaaagtgctgagattacaggcgtgagccaccgcgccagggcccaaagtcaaaccctcccacctgatctggctgctgcttccctgctgtctccttgtgggaaaagaacctgcagtcctgtgtctaggccagtcctgcccctcagtcaagcgaggcgcctttgcccctgccctcatcagcagtccccggggctccgctggttaacagcgcaggaagccgcggccccacgcagacctgggctccgggccctccgccagctgcagttccagatcccgccgaaggagggggcgggcggagcgcgggtggggcggggcccggctctccgggtgggcggggcggggcggggccgggctggggcgggggtgtgactgcgcatgcccacctgtggccggcatccctgccgcccaggtgcagctgactgcacgtgcagctgaattcacaccaggtttttgtttttgttttttgagacggagtcttgctctgtcccccaggctggagtgcagtgttgcaatctcggctcactgcaacctccacctcccaggttcaagcgattctgcctcagcctcctgagtagctgggattacaggtgcgcaccaccacgcctggctaattttgtatttttagtagagatggggtttcaccgtgttggccagtctggtctcgaacccctgacctcaagtgatgcgcccgcctcagcctctcaatgtgctgggattacaggcgtgagcccccgcgccggcccaggcccatgtttttaaagcccacacctgcctcctttgcccagtggtctcacttcagcacggcctcagggctgactcagtctctccggagagtggggcgagcccagcctctcctacagaacctcttcttccccagcagaagaggaggggctgggaggctgagctcccgcctctgaccgcctgtctgtctctcttggtcaccagctgtgtgtccttctccatctacacggcctggggcgagcactgtgagcacctgagcatgaaactcgacgcgttcttcggcatcttctttggggccctgggcggcctcttgctgctgggggtcgggacgttcgtggtcctgcgcttctggggttgctccggggccaggttctcctatttcctgaactcagctgaggccttgccttgaaggggcagctgtggcctaggctacctcaagactcacctcatccttaccgcacatttaaggcgccattgcttttgggagactggaaaagggaaggtgactgaaggctgtcaggattcttcaaggagaatgaatactgggaatcaagacaagactataccttatccataggcgcaggtgcacagggggaggccataaagatcaaacatgcatggatgggtcctcacgcagacacacccacagaaggacactagcctgtgcacgcgcgcgtgcacacacacacacacacacacgagttcataatgtggtgatggccctaagttaagcaaaatgcttctgcacacaaaactctctggtttacttcaaattaactctatttaaataaagtctctctgactttttgtgtctccaaaa

SEQ ID NO: 49 - DNA, 12708 bp, Homo sapiens
ctggtgtttccatcttcagaggggagtccgaggatactgtggaagctgaggtagcactgctgacagcaagaggggtggcgtgacctgtggatactgaggaaaggctggtgacaggaagaggggtggcgtgacctgtggatgctgaggaagtgtcggtgacaggaagaggggtggcgtgacctgtggatgctgaggaagtgtcggtgacaggaagaggggtggtgtcacctgtggatgctgaggaagtgctggtgacaggaagaggggtgccgtgacctgtggacactgaggaagcgtcggtgacaggaagagaggtggtgtgacctgaggatgctgaggaagggatggtgacaggaagaggcgtggtgtcacctgtggatactgaggaaaggctggtgacaggaagaggggtggcctgacctgtggatgcagaggaagtgtcggtgacaggaagaggcgtggtgtcacctgtggatactgaggaaaggctggtgagaggaagaggggtggcgtgaccggtggatgctgaggaagcatcggtgacaggaagagtgctggtgtcacctctggatgctgaggaagggctggtgacatgaagaggggtggcgtgacctgtggataatgaggaagcattggtgacaggaagaggggtggtgtcacctgtggatgctgaggaagtgctggtgacaggaacaggggtggcgtgacctgtggatgctgaggaagggctggtgacaggaagaggggtggcgtgacctgtagatactgaggaagtgctggtgacaggaagaggggtggcgtgacctgtggatactgaggaagtgtcggtgacaggaaggggggtggcgtgacctgtggatgctgaggaacggctggtgacaggaagagaggtggcgtgacctgtggatactgaggaagtgtcggtgacaggaagaggggtggtgtcacctgtggatgctgaggaagtgctggtgacaggaagaggggtggcatgtcctgtggatgccgaggaaacgtcggtgacaggaagacgggtggtgtcatctgtggaagctgaggaagtgtcggtgacaggaagaggggtggcgtgacgtgtggatgctgaggaagtgtcggtgacaggaagaggggtggtgtcacctgtggaagctgaggaaaggccggtaacaggaagaggggtggcgtgacctgtggatgctgaggaagggctagtgacaggaagaggcatggtgtcacctgtggatactgaggaagggatggtgacaggaagaggggtggcgtgacctgtggatgctgaggaagcgtcggtgacaggaagaggggtggtgtcacctgtggatactgaggaaaggctggtgacaggaagaggggtggcctgacctgtggatgctgaggaagtgtcggtgacaggaagaggggtggtgtcacctgtggatgctgaggaagcgtcggtgacaggaagaggcgtggcgtgacctgtggacactgaggaagcgtcggtgacaagaagaggggtggcgtgacctgtggatgctgaggaagtgctggtgacaggaagaggggtgacgtgacctgtggatgctgaggaagggctggtgacatgaagaggggtgacgtgacctgtagatactgaggaagtgctggtgacaggaagaggggtggtgtgacctgaggatgatgaggaagggatggtgacaggaagaggggtggcctgacctgtggatgctgaggaagtgtccgtgacaggaagacgggtggtgtcacctgtggatgctgaggaagtgtcggtgacaggaagaggggtggcgtgacctgtggatactgaggaagcgtcggtgacaagaagaggggtggtgtcacctgtggatactgaggaaaggctggtgacaggaagaggggtggcctgacctgtggatgctgaggaagtgtccgtgacaggaagacgggtggtgtcacctgtggatgctgaggaagtgtcggtgacaggaagaggggtggcgtgacctgtggatactgaggaagcgtcggtgacaagaagaggggtggtgtcacctgtggatactgaggaaaggctggtgacaggaagaggggtggcctgacctgtggatgccgaggaagcgtcggtgacaggaagaggggtggtgtcacctgtggatactgaggaaaggctggtgacaggaagaggcgtggcgtgaccggtggatactgaggaagtgtcggtgacaggaagaggggtggcgtgaccggtggatgctgaggaagcgccggtgacaggaagagtgctggtgtcacctgtggatgctgaggaagggatggtgacatgaagaggggtggtgtgacctgtagatgctgaggaagggctggtgacaggaagaggggtggtgtcacctgtggatgctgaggaagtgtcggtgacaggaagaggggtggtgtgacctgtagatgctgaggaagtgctggtgacaggaacaggggtggcgtgaccggtggatgctgaggaagtgctggtgacaggaagaggggtggcgtgacctgtggatgctgaggaagggctagtgacaggaagaggcatggtgtcacctgtggatactgaggaagtgttggtgacaggaagaggggtggcctgacctgtggatgccgaggaaatgtcggtgacaggaagacgggtggtgtcacctgtggaagctgaggaaaggccggtgacaggaagaggggtggcgtgacctgtggatactgaggaagtgtcggtgacaggcacaggggtggtgtcacctgtggatgctgaggaagggctggtgacatgaagaggggtggcgtgacctgtggatgctgaggaagcgtcggtgacaagaagaggagtggcgtgacctgtggatactgaggaagtctcggtgacaagaagaggggtggtgtcacctgtggatgatgaggaagtgtcggtgacaggaagagaggtggtgtcacctgtgtatgctgaggaagtgtcggtgacaggaagagaggtggtgtcacctgtggatgctgaggaagtgtcggtgacaggaagagaggtggcatgaccggtggatgctgaggaagggctagtgacaggaagaggcgtggtgtcacctgtggatactgaggaaaggctggtgacaggaagaggggtggcctgacctgtggatgctgaggaagcgtcggtgacaagaagaggagtggcgtgacctgtggatgctgaggaagggctagtgacaggaagaggcgtggtgtcacctgtggatactgaggaaaggctggtgacaggaagaggggtggcctgacctgtggatgctgaggaagtgtcggtgacaggaagaggggtggtgtcacctgtggatgctgaggaagtgctggtgacaggaagagcggtggcctgacctgtggatgctgaggaagtgtcggtgacaggaagaggggtggtgtgacctgtggatgctgaggaagggctagtgacaggaagaggcgtggtgtcacctgtggatactgaggaaaggctggtgacaggaagaggggtggcgtgacctgtggatgctgaggaagtgtcggtgacaggaagcggggtggcgtgaccggtggatgctgaggaagggctggtgacatgaagagggttggcgtgacctgtggatgctgaggaagtgtcggtgacaggaagcggggtggcgtgaccggtggatgctgaggaagggctggtgacatgaagaggggtggcgtgacctgtggatattgaggaagtgtcggtgacaggaagagaggtggcgtgacctatggatgctgaggaagtgtcggtgacaggaagaagggtggcgtgacctgtggatgctgaggaagtgtcggtgtcaggaagaggggtggcgtgacctgtggatgctgaggaagtgtcggtgacaggaagagaggtggcgtgacctgtggatgctgaggaagtgtcggtgacaggaagaggggtggtgtcacctgtggatactgaggaaaggctggtgacaggaagaggggtggcctgacctgtggatgctgaggaagtgtcggtgacaggaagaggcgtggtgtcacctgtggatactgaggaaaggctggtgagaggaagaggggtagcgtgacctgtggacactgaggaagcgtcggtgacaggaagaggggtggcatgacctgtggacactgaggaagcgtcggtgacaggaagagaggtggcgtgacctgtggacactgaggaagcgtcggtgacaggaagaggggtggtgtgacctgaggatgctgaggaagggatggtgacaggaagagaggtggcatgacctgtgaacactgaggaagcgtcggtgacaggaagagaggtggcgtgacctgtggacactgacgaagcgtcggtgacaggaagaggggtggtgtgacctgtggatgctgaggaagggctggtgacatgaagaggggtggcgtgacctgtggatactgaggaagtgttggtgacaggaagaggggtggcgtgacctgtggatgctgaggaagtgtcggtgacaggaagaggggtggtgtcacctgtggatactgaggaagtctcggtgacaagaagaggggtggtgtcacctgtggatgatgaggaagtgtcggtgacaggaagagaggtggtgtcacctgtgtatgctgaggaagtgtcggtgacaggaagagaggtggtgtcacctgtggatgctgaggaagtgtcggtgacaggaagagaggtggcatgaccggtggatgctgaggaagggctagtgacaggaagaggcgtggtgtcacctgtggatactgaggaaaggctggtgacaggaagaggggtggcctgacctgtggatgctgaggaagcgtcggtgacaagaagaggagtggcgtgacctgtggatgctgaggaagggctagtgacaggaagaggcgtggtgtcacctgtggatactgaggaaaggctggtgacaggaagaggggtggcctgacctgtggatgctgagtaagtgtcggtgacaggaagaggggtggtgtcacctgtggatgctgaggaagtgctggtgacaggaagaggggtggcgtgacctgtggatgctgcggaagtgtcggtgacaggaagagaggtggcgtgacctgtggatgctgaggaagggctggtgacatgaagagaggtggcgtgacgtgtggataatgaggaagcattggtgacaggaagaggggtggtgtcacctgtggatgctgagggagtgtcggtgacaggtagaggggtggtgtgacctgtagatgctgaggaagggctggtgacaggaagacgggtggtgtcacctgtggatactgacgaagcgtcggtgacaagaagaggggtggtgtgacctgtggatactgaggaagtgtcggtgccaggaagaggggtggtgtcacctgtggatgctgaggaagtgctggtgacaggaagaggggtggcatgacctgtggatgccgaggaaacgttggtgacaggaagacgggtggtgtcacctgtggaagctgaggaaaggccggtgacaggaagatgggtggcgtgacctgtggatgctgaggaagtgtcggtgacaggaagaggggtggtgtcacctgtggatgctgaggaagcgtcggtgacaggaagaggggtggtgtgacctgaggatgctgaggaagagctggtgacaggaagaggggtggtgtcacctgtggatactgaggaagcgtcggtgacatgaagaggggtggcgtgacctgtggatgctgaggaagggctagtgacaggaagaggcgtggtgtcacctgtggatgctgaggaaaggctggtgacaggaagaggggtggcgtgacctgtggatgctgaggaagcgtcggtgacatgaagaggggtggcgtgacctgtggatgctgaggaagggctagtgacaggaagaggagtggtgtcacctgtggatactgaggaaaggctggtgacaggaagagaggtggcgtgacctgtggacactgaggaagcgtcggtgacaggaagaggggtggtgtcacctgtggatgctgaggaaaggctggtgacaggaagaggggtggcctgtcctgtggatgctgaggaagtgtcggtgacaagaagagggatggcgtgacctgtggatgctgaggaagggctggtgacaggaagaggggtggcctgacctgtggatgctgaggaagtgtcggtgacaggaagaggggtggcgtgacctgtggatgctgaggaagtgtcggtgacaggaagagaggtggcgtgacctgtggatgctgaggaagtgtcggtgacaggaagagaggtggcgtgacctgtggatactgaggaagtttcggtgacaggaagaggggtggtgtcacctgtggatgttgaggaagggctggtgacaggaagaggggtggtgtcccctgtggataatgaggaagcatcggtgtcatgaagagcggtggcgtgacctgtggatactgaggaagcgtcggtgacaagaagagaggtggcgtgacctgtggatattgaggaagcgtcggtgacaagaagaggggtggcgtgacctgtggatgctgaggaagggttagtgacaggaagaggcgtggtgtcacctgtggatactgaggaaaggctggtgacaggaagaggggtgtcctgacctgtggatgctgaggaagtatcggtgacaggaagcggcgtggtgtcaccagtggatgctgaggaaaggctggtgacaggaagaggggtggcctgtcctgtagatactgaggaagtgtcggtgaccggaagaggggtggcatgacctgtggacactgaggaagcgtcggtgacaggaagaggggtggcgtgacctgtggacactgaggaagcgtcggtgacaggaagagaggtggcgtgacctgtgggtactgaggaagcgtcggtgacaggaagaggggtggtgtgacctgaggatgctgaggaagggctagtgacaggaagaggcgtggtgtcacctgtggatgctgaggaaaggctggtgacaggaagagaggtggcgtgacctgtggatactgaggaagcgtcggtgacatgaagaggggtggtgtcacctgtggatgctgagttagtgtcggtgacaggaagaggggtggtgtcacctgtggatactgaggaagcgtcggtgacaagaagagaggtggcgtgacctgtggacactgaggaagcgtcggtgacaggaagagaggtggtgtgacctgaagatgctgaggaagggatggtgacaggaagagaggtggtgtcacctgtggatgctgaggaagcgtcggtgacaggaagaggggtggtgtcacctgtggatgctgaggaagggctggtgacaggaagagggatggcctgacctgtggatgccgaggaaacgtcggtgacaggaagacgggtggtgtcatctgtggaagctgaagaaaggccggtgacaggaagtggggtggcgtgagctgtggatactgaggaagtgtcggtgacaggaagaggggtggcctgacctgtggatgctgaggaagcgtcagtgacaagaagagggctggcgtgacctgtggatgctgaggaagggctagtgacaggaagaggcgtggtgtcacctgtggatactgaggaaaggctggtgacaggaagaggggtggcctgacctgtggatgccgaggaaacgtcggtgacaggaagacgggtggtgtcatctgtggtagctgaggaaaggccggtgacaggaagaggggtggcgtgaccggtggatgctgaggaagtgctggtgacaggaagaggggtggcgtgacctgtggatgctgaggaagggctggtgacatgaagaggggtggcgtgacctgtggataatgaggaagcattggtgacaggaagaggggtggtgtcacctgtggatgctgaggaagtgtcggtgacaggaagaggggtggtgtgacctgtagatgctgaggaagggctggtgacaggaagaggggtggtgtcacctttggatgctgaggaagtgtcggtgacaggaagaggggtggtgtgacctgtagatgctgaggaagggctggtgacaggaagaggggtggtgtgacctgtggatactgaggaagcgtcggtgacaggaagaggggtggtgtcacctgtggatgctgaggaagggctagtgacaggaagaggcatggtgtcacctgtggatgctgaggaaaggctggtgacaggaagaggggtggcgtgacctgtggatgctgaggaagggctggtgacatgaagaggagtgacgtgacctgtggatgctgaggaagtgctagtgacaggaagaggcgtggtgtcacctgtggatactgaggaagtgtcggtgacaagaagagaggtggcctgacctgtggatgctgaggaagtgtcggtgacaggaagaggggtggtgtgacctgtggatgctgcggaagggatggttacaggaagagaggtggcgtgatctgtggacactgaggaagcgtcggtgacaggaagaggggtggcgtgtcctgtggatgctgaggaagtgtcggtgacaagaagaggggtggcgtgacctgtggatgctgaggaagggctagtgacaggaagaggcgtggtgtcacctgtggatactgaggaaaggctggtgacaggaagaggggtggcctgacctgtggatgctgaggaagcatcagtgacatgaagaggggtggtgtgacctgtggatactgaggaagcgtcggtgacaggaagaggggtggtgtcacctgtggatgctgaggaagggctagtgacaggaagaggcatggtgtcacctgtggatgctgaggaaaggctggtgacaggaagaggggtggcgtgacctgtggatgctgaggaagggctggtgacatgaagaggagtgacgtgacctgtggatgctgaggaagtgctagtgacaggaagaggcgtggtgtcacctgtggatactgaggaagtgtcggtgacaagaagagaggtggcctgacctgtggatgctgaggaagtgtcggtgacaggaagaggggtggtgtgacctgtggatgctgcggaagggatggttacaggaagagaggtggcgtgatctgtggacactgaggaagcgtcggtgacaggaagaggggtggcgtgtcctgtggatgctgaggaagtgtcggtgacaagaagaggggtggcgtgacctgtggatgctgaggaagggctagtgacaggaagaggcgtggtgtcacctgtggatactgaggaaaggctggtgacaggaagaggggtggcctgacctgtggatgctgaggaagcatcagtgacatgaagaggggtggtgtgacctgtggatactgaggaagtgtcggtgacaggaagagaggtggcgtgacctgtggatgctgaggaagtgtcggtgacaggaagaggggtggtgtgacctgtggatactgaggaagtgtcggtgacaggaagagaggtggcgtgacctgtggatgctgaggaagtgtcagtgacaggaagaggggtggtgtcacctgtggatgctgaggaaaggctggtgacaggaagaggggtgacgtgacctgtggatgctgaggaagtgtcagtgacaggaagaggggtgctgtcacctgtggatgctgaggaaaagctggtgacaggaagaggggtgacgtgacctgtggattctgaggaagtgtcggtgacaggaagaggggtggtgtgacctgtggatactgaggaagggctggtgacaggaagaggggtggcgtgacctgtggatactgaggaagcatcggtgacatgaagaggggtggtgtgacctgtggatgccgaggaagcgtaggtgacaggaagaggggtggcgttgctgatgagggccgtggtgaaggttttaccagaccctgaaggtgacagagtgtgggtctcggtttgtggagatgtaagcccagtggatgtgatcgatggaggtgtgggtgggactgttgagaaggtgtcggttgcctgggacgccaggctgatagtgtcagaccctctgctggttcttgtcctctgagtctgggccatccgggaaatggcggctgtctcctgaggagaggcactgggagaagttgggcttgactgtcctgtcggtctccctgcagtggaggcctcagagagggtgggatgaaaggtgccggggacgatcgaagacgccattcctgtgcttactgggatggcaccatgactggctgaggcggacagcaattcggttgttgactgggttgtgtgactgtccctggaggggtttgatgaaaaccttgtcgtctctcctgaggtggatattccttcgcttcctgaaggtgttgtgccactcgccccggatgaggaaggggtagctgtgcccgctgaggtggttcgtgaccctgaggaggccggttcgctggtctgtgtttgtccagaggcctctgtgctctcagcctggtgggtatgggtcatggctgctgctgtgtcaggtgaggtgctggcagaggctgatgtccattgtccttctgggccccctggtgttgacactacagagttggccagagtaagggcacctgttttggaaagtgacgtgcctcctgagggccccagggtggcatcatggctgctgggtgctgcctgcagtgctgtggtcggggcctgggttgtgtgaccatccccggtgggagctggggcaaaggttgttgctgcacttatggtgggtgcgtcctgaggaacaatttctgagggcgagtgcccactggctgtgaaggaagaacctggggtggtgactgtcttggtgtcagtcatgggggagacggacctcgtggtttgtgattcttgtgtggtctgcggggcttgagtgtgacccctttgggaaacagctggtgattcctgaggagaggtgcttgtggaatgtattgttgaatgatttgttgatggtgccgttgtaatttgttgggatgtgtgtctatccagcataggtgaagaagatggggatgtggccgtttttatcatctgagtcacactgtagcttgggctgctgagaagagcctctccagtggtccccgtttcttgtgtccattgtgtctgggcgcctgcccctgttgtttttgggagagttgtgctgtgggaggagtatgtggtgggctctcctggttcccctattgctgagaccttagaggggacccttggaatagtgccagctgtccctgtagatggatttcctgtgacaagcctagtggcagcacctgttgatgttgagggcagttctgtttgtgaccaagttgtgtggcctttgctggagaatgaggaaggccatgttgttgtttcatgtagagtaaatatttcttgagacacacctggagagaatgagctcctctcatgaggccgtcctgtggtctctgcaccttcactctgctgggtgtggaaagctgtggatatttttggaggtagagaactgggggagagtgctgttgacagagtgtctgaccaccatatggttgaaactttggaagtgattgcagaaatggttccacttacagatagtgatgtctcctctgtgtttccaagagtagagtctctggaggttggcattctgaacacctttgatgttaccaggaatgtattgctgacactggaaggggatgaggtggttgtttcaccagaagggaatgtctcctgagaaactgttccacttgggttgaatccacttggtgaggataaaacagttgatgttgtaaccggtgtgagggtgttgagggtgttgatttgagatactctggtggtctccacgctctgagtctggtggttcttagaaaaagctgttgtgtcctgagtagaagtccttgagaaagttgctggtgattgtccttctggatcaaatgttactaaggctgctgaggtgactggcataagacttccagtaacaggtactgatgatgtctcccctgggtttccaagagtggagcctgtggaggttgtcactgttatcttctctgatgtcatcatggatgtgtttgtgacactacagagggatgaggtagctgtttcacctgaaggagatgtcttctgagaaacagtccctgtcacattgtgtacacttggagagaaagaaggagttgaagtggttctgtgtgttagggtgctggtttgagattccctggtggtctgcgtgctccgagtccagtggttctgagaagaagctgatgtgtcttggatagaggtcctccaggaagttgttcgtgattgtccttcctgtccagctgttattgagactgctgaggtcactggggtagaactttcagttcctgctgttgatgtctcttctgtgtttccaagagtggagtctgtggaggttgtcattgttatagtctttgatgtcatcatgagtgtgttggtgacactggagggaaatgatgtggtcatttcatctggaggagctgtctccatcacattgtgtacacttggggaagaaaaaagagttgatgtcatcatctgcgtgagggtgtcggtttgagctttgctggtggtctccgtgctcttagtctggtggttctgagatgaagctgatatgtcctgattagaggtccttgaagaagctgcagttgattgtccctctagtgtcgctgttgttgagcctgttgaggtgactggggcagcagttttacttccagttattaatgtgtcctctgtggttc

SEQ ID NO: 50 - DNA, 9408 bp, Homo sapiens
tgtcacctgtggatgctgaggaagtgctggtgacaggaagaggggtgccgtgacctgtggacactgaggaagcgtcggtgacaggaagagaggtggtgtgacctgaggatgctgaggaagggatggtgacaggaagaggcgtggtgtcacctgtggatactgaggaaaggctggtgacaggaagaggggtggcctgacctgtggatgcagaggaagtgtcggtgacaggaagaggcgtggtgtcacctgtggatactgaggaaaggctggtgagaggaagaggggtggcgtgaccggtggatgctgaggaagcatcggtgacaggaagagtgctggtgtcacctctggatgctgaggaagggctggtgacatgaagaggggtggcgtgacctgtggataatgaggaagcattggtgacaggaagaggggtggtgtcacctgtggatgctgaggaagtgctggtgacaggaacaggggtggcgtgacctgtggatgctgaggaagggctggtgacaggaagaggggtggcgtgacctgtagatactgaggaagtgctggtgacaggaagaggggtggcgtgacctgtggatactgaggaagtgtcggtgacaggaaggggggtggcgtgacctgtggatgctgaggaacggctggtgacaggaagagaggtggcgtgacctgtggatactgaggaagtgtcggtgacaggaagaggggtggtgtcacctgtggatgctgaggaagtgctggtgacaggaagaggggtggcatgtcctgtggatgccgaggaaacgtcggtgacaggaagacgggtggtgtcatctgtggaagctgaggaagtgtcggtgacaggaagaggggtggcgtgacgtgtggatgctgaggaagtgtcggtgacaggaagaggggtggtgtcacctgtggaagctgaggaaaggccggtaacaggaagaggggtggcgtgacctgtggatgctgaggaagggctagtgacaggaagaggcatggtgtcacctgtggatactgaggaagggatggtgacaggaagaggggtggcgtgacctgtggatgctgaggaagcgtcggtgacaggaagaggggtggtgtcacctgtggatactgaggaaaggctggtgacaggaagaggggtggcctgacctgtggatgctgaggaagtgtcggtgacaggaagaggggtggtgtcacctgtggatgctgaggaagcgtcggtgacaggaagaggcgtggcgtgacctgtggacactgaggaagcgtcggtgacaagaagaggggtggcgtgacctgtggatgctgaggaagtgctggtgacaggaagaggggtgacgtgacctgtggatgctgaggaagggctggtgacatgaagaggggtgacgtgacctgtagatactgaggaagtgctggtgacaggaagaggggtggtgtgacctgaggatgatgaggaagggatggtgacaggaagaggggtggcctgacctgtggatgctgaggaagtgtccgtgacaggaagacgggtggtgtcacctgtggatgctgaggaagtgtcggtgacaggaagaggggtggcgtgacctgtggatactgaggaagcgtcggtgacaagaagaggggtggtgtcacctgtggatactgaggaaaggctggtgacaggaagaggggtggcctgacctgtggatgctgaggaagtgtccgtgacaggaagacgggtggtgtcacctgtggatgctgaggaagtgtcggtgacaggaagaggggtggcgtgacctgtggatactgaggaagcgtcggtgacaagaagaggggtggtgtcacctgtggatactgaggaaaggctggtgacaggaagaggggtggcctgacctgtggatgccgaggaagcgtcggtgacaggaagaggggtggtgtcacctgtggatactgaggaaaggctggtgacaggaagaggcgtggcgtgaccggtggatactgaggaagtgtcggtgacaggaagaggggtggcgtgaccggtggatgctgaggaagcgccggtgacaggaagagtgctggtgtcacctgtggatgctgaggaagggatggtgacatgaagaggggtggtgtgacctgtagatgctgaggaagggctggtgacaggaagaggggtggtgtcacctgtggatgctgaggaagtgtcggtgacaggaagaggggtggtgtgacctgtagatgctgaggaagtgctggtgacaggaacaggggtggcgtgaccggtggatgctgaggaagtgctggtgacaggaagaggggtggcgtgacctgtggatgctgaggaagggctagtgacaggaagaggcatggtgtcacctgtggatactgaggaagtgttggtgacaggaagaggggtggcctgacctgtggatgccgaggaaatgtcggtgacaggaagacgggtggtgtcacctgtggaagctgaggaaaggccggtgacaggaagaggggtggcgtgacctgtggatactgaggaagtgtcggtgacaggcacaggggtggtgtcacctgtggatgctgaggaagggctggtgacatgaagaggggtggcgtgacctgtggatgctgaggaagcgtcggtgacaagaagaggagtggcgtgacctgtggatactgaggaagtctcggtgacaagaagaggggtggtgtcacctgtggatgatgaggaagtgtcggtgacaggaagagaggtggtgtcacctgtgtatgctgaggaagtgtcggtgacaggaagagaggtggtgtcacctgtggatgctgaggaagtgtcggtgacaggaagagaggtggcatgaccggtggatgctgaggaagggctagtgacaggaagaggcgtggtgtcacctgtggatactgaggaaaggctggtgacaggaagaggggtggcctgacctgtggatgctgaggaagcgtcggtgacaagaagaggagtggcgtgacctgtggatgctgaggaagggctagtgacaggaagaggcgtggtgtcacctgtggatactgaggaaaggctggtgacaggaagaggggtggcctgacctgtggatgctgaggaagtgtcggtgacaggaagaggggtggtgtcacctgtggatgctgaggaagtgctggtgacaggaagagcggtggcctgacctgtggatgctgaggaagtgtcggtgacaggaagaggggtggtgtgacctgtggatgctgaggaagggctagtgacaggaagaggcgtggtgtcacctgtggatactgaggaaaggctggtgacaggaagaggggtggcgtgacctgtggatgctgaggaagtgtcggtgacaggaagcggggtggcgtgaccggtggatgctgaggaagggctggtgacatgaagagggttggcgtgacctgtggatgctgaggaagtgtcggtgacaggaagcggggtggcgtgaccggtggatgctgaggaagggctggtgacatgaagaggggtggcgtgacctgtggatattgaggaagtgtcggtgacaggaagagaggtggcgtgacctatggatgctgaggaagtgtcggtgacaggaagaagggtggcgtgacctgtggatgctgaggaagtgtcggtgtcaggaagaggggtggcgtgacctgtggatgctgaggaagtgtcggtgacaggaagagaggtggcgtgacctgtggatgctgaggaagtgtcggtgacaggaagaggggtggtgtcacctgtggatactgaggaaaggctggtgacaggaagaggggtggcctgacctgtggatgctgaggaagtgtcggtgacaggaagaggcgtggtgtcacctgtggatactgaggaaaggctggtgagaggaagaggggtagcgtgacctgtggacactgaggaagcgtcggtgacaggaagaggggtggcatgacctgtggacactgaggaagcgtcggtgacaggaagagaggtggcgtgacctgtggacactgaggaagcgtcggtgacaggaagaggggtggtgtgacctgaggatgctgaggaagggatggtgacaggaagagaggtggcatgacctgtgaacactgaggaagcgtcggtgacaggaagagaggtggcgtgacctgtggacactgacgaagcgtcggtgacaggaagaggggtggtgtgacctgtggatgctgaggaagggctggtgacatgaagaggggtggcgtgacctgtggatactgaggaagtgttggtgacaggaagaggggtggcgtgacctgtggatgctgaggaagtgtcggtgacaggaagaggggtggtgtcacctgtggatactgaggaagtctcggtgacaagaagaggggtggtgtcacctgtggatgatgaggaagtgtcggtgacaggaagagaggtggtgtcacctgtgtatgctgaggaagtgtcggtgacaggaagagaggtggtgtcacctgtggatgctgaggaagtgtcggtgacaggaagagaggtggcatgaccggtggatgctgaggaagggctagtgacaggaagaggcgtggtgtcacctgtggatactgaggaaaggctggtgacaggaagaggggtggcctgacctgtggatgctgaggaagcgtcggtgacaagaagaggagtggcgtgacctgtggatgctgaggaagggctagtgacaggaagaggcgtggtgtcacctgtggatactgaggaaaggctggtgacaggaagaggggtggcctgacctgtggatgctgagtaagtgtcggtgacaggaagaggggtggtgtcacctgtggatgctgaggaagtgctggtgacaggaagaggggtggcgtgacctgtggatgctgcggaagtgtcggtgacaggaagagaggtggcgtgacctgtggatgctgaggaagggctggtgacatgaagagaggtggcgtgacgtgtggataatgaggaagcattggtgacaggaagaggggtggtgtcacctgtggatgctgagggagtgtcggtgacaggtagaggggtggtgtgacctgtagatgctgaggaagggctggtgacaggaagacgggtggtgtcacctgtggatactgacgaagcgtcggtgacaagaagaggggtggtgtgacctgtggatactgaggaagtgtcggtgccaggaagaggggtggtgtcacctgtggatgctgaggaagtgctggtgacaggaagaggggtggcatgacctgtggatgccgaggaaacgttggtgacaggaagacgggtggtgtcacctgtggaagctgaggaaaggccggtgacaggaagatgggtggcgtgacctgtggatgctgaggaagtgtcggtgacaggaagaggggtggtgtcacctgtggatgctgaggaagcgtcggtgacaggaagaggggtggtgtgacctgaggatgctgaggaagagctggtgacaggaagaggggtggtgtcacctgtggatactgaggaagcgtcggtgacatgaagaggggtggcgtgacctgtggatgctgaggaagggctagtgacaggaagaggcgtggtgtcacctgtggatgctgaggaaaggctggtgacaggaagaggggtggcgtgacctgtggatgctgaggaagcgtcggtgacatgaagaggggtggcgtgacctgtggatgctgaggaagggctagtgacaggaagaggagtggtgtcacctgtggatactgaggaaaggctggtgacaggaagagaggtggcgtgacctgtggacactgaggaagcgtcggtgacaggaagaggggtggtgtcacctgtggatgctgaggaaaggctggtgacaggaagaggggtggcctgtcctgtggatgctgaggaagtgtcggtgacaagaagagggatggcgtgacctgtggatgctgaggaagggctggtgacaggaagaggggtggcctgacctgtggatgctgaggaagtgtcggtgacaggaagaggggtggcgtgacctgtggatgctgaggaagtgtcggtgacaggaagagaggtggcgtgacctgtggatgctgaggaagtgtcggtgacaggaagagaggtggcgtgacctgtggatactgaggaagtttcggtgacaggaagaggggtggtgtcacctgtggatgttgaggaagggctggtgacaggaagaggggtggtgtcccctgtggataatgaggaagcatcggtgtcatgaagagcggtggcgtgacctgtggatactgaggaagcgtcggtgacaagaagagaggtggcgtgacctgtggatattgaggaagcgtcggtgacaagaagaggggtggcgtgacctgtggatgctgaggaagggttagtgacaggaagaggcgtggtgtcacctgtggatactgaggaaaggctggtgacaggaagaggggtgtcctgacctgtggatgctgaggaagtatcggtgacaggaagcggcgtggtgtcaccagtggatgctgaggaaaggctggtgacaggaagaggggtggcctgtcctgtagatactgaggaagtgtcggtgaccggaagaggggtggcatgacctgtggacactgaggaagcgtcggtgacaggaagaggggtggcgtgacctgtggacactgaggaagcgtcggtgacaggaagagaggtggcgtgacctgtgggtactgaggaagcgtcggtgacaggaagaggggtggtgtgacctgaggatgctgaggaagggctagtgacaggaagaggcgtggtgtcacctgtggatgctgaggaaaggctggtgacaggaagagaggtggcgtgacctgtggatactgaggaagcgtcggtgacatgaagaggggtggtgtcacctgtggatgctgagttagtgtcggtgacaggaagaggggtggtgtcacctgtggatactgaggaagcgtcggtgacaagaagagaggtggcgtgacctgtggacactgaggaagcgtcggtgacaggaagagaggtggtgtgacctgaagatgctgaggaagggatggtgacaggaagagaggtggtgtcacctgtggatgctgaggaagcgtcggtgacaggaagaggggtggtgtcacctgtggatgctgaggaagggctggtgacaggaagagggatggcctgacctgtggatgccgaggaaacgtcggtgacaggaagacgggtggtgtcatctgtggaagctgaagaaaggccggtgacaggaagtggggtggcgtgagctgtggatactgaggaagtgtcggtgacaggaagaggggtggcctgacctgtggatgctgaggaagcgtcagtgacaagaagagggctggcgtgacctgtggatgctgaggaagggctagtgacaggaagaggcgtggtgtcacctgtggatactgaggaaaggctggtgacaggaagaggggtggcctgacctgtggatgccgaggaaacgtcggtgacaggaagacgggtggtgtcatctgtggtagctgaggaaaggccggtgacaggaagaggggtggcgtgaccggtggatgctgaggaagtgctggtgacaggaagaggggtggcgtgacctgtggatgctgaggaagggctggtgacatgaagaggggtggcgtgacctgtggataatgaggaagcattggtgacaggaagaggggtggtgtcacctgtggatgctgaggaagtgtcggtgacaggaagaggggtggtgtgacctgtagatgctgaggaagggctggtgacaggaagaggggtggtgtcacctttggatgctgaggaagtgtcggtgacaggaagaggggtggtgtgacctgtagatgctgaggaagggctggtgacaggaagaggggtggtgtgacctgtggatactgaggaagcgtcggtgacaggaagaggggtggtgtcacctgtggatgctgaggaagggctagtgacaggaagaggcatggtgtcacctgtggatgctgaggaaaggctggtgacaggaagaggggtggcgtgacctgtggatgctgaggaagggctggtgacatgaagaggagtgacgtgacctgtggatgctgaggaagtgctagtgacaggaagaggcgtggtgtcacctgtggatactgaggaagtgtcggtgacaagaagagaggtggcctgacctgtggatgctgaggaagtgtcggtgacaggaagaggggtggtgtgacctgtggatgctgcggaagggatggttacaggaagagaggtggcgtgatctgtggacactgaggaagcgtcggtgacaggaagaggggtggcgtgtcctgtggatgctgaggaagtgtcggtgacaagaagaggggtggcgtgacctgtggatgctgaggaagggctagtgacaggaagaggcgtggtgtcacctgtggatactgaggaaaggctggtgacaggaagaggggtggcctgacctgtggatgctgaggaagcatcagtgacatgaagaggggtggtgtgacctgtggatactgaggaagcgtcggtgacaggaagaggggtggtgtcacctgtggatgctgaggaagggctagtgacaggaagaggcatggtgtcacctgtggatgctgaggaaaggctggtgacaggaagaggggtggcgtgacctgtggatgctgaggaagggctggtgacatgaagaggagtgacgtgacctgtggatgctgaggaagtgctagtgacaggaagaggcgtggtgtcacctgtggatactgaggaagtgtcggtgacaagaagagaggtggcctgacctgtggatgctgaggaagtgtcggtgacaggaagaggggtggtgtgacctgtggatgctgcggaagggatggttacaggaagagaggtggcgtgatctgtggacactgaggaagcgtcggtgacaggaagaggggtggcgtgtcctgtggatgctgaggaagtgtcggtgacaagaagaggggtggcgtgacctgtggatgctgaggaagggctagtgacaggaagaggcgtggtgtcacctgtggatactgaggaaaggctggtgacaggaagaggggtggcctgacctgtggatgctgaggaagcatcagtgacatgaagaggggtggtgtgacctgtggatactgaggaagtgtcggtgacaggaagagaggtggcgtgacctgtggatgctgaggaagtgtcggtgacaggaagaggggtggtgtgacctgtggatactgaggaagtgtcggtgacaggaagagaggtggcgtgacctgtggatgctgaggaagtgtcagtgacaggaagaggggtggtgtcacctgtggatgctgaggaaaggctggtgacaggaagaggggtgacgtgacctgtggatgctgaggaagtgtcagtgacaggaagaggggtgc

SEQ ID NO: 51 - DNA, 51 bp, Homo sapiens
gacctgtggatgctgaggaagtgtcagtgacaggaagaggggtgctgtcac

SEQ ID NO: 52 - DNA, 51 bp, Homo sapiens
ccccagcagcaagaggccgcccagggccccaaagaagatgccgaagaacgc

SEQ ID NO: 53 - DNA, 51 bp, Homo sapiens
gtgaccggtggatgctgaggaagcgccggtgacaggaagagtgctggtgtc

SEQ ID NO: 54 - DNA, 51 bp, Homo sapiens
gaggaaaggccggtgacaggaagaggggtggcgtgaccggtggatgctgag

SEQ ID NO: 55 - DNA, 51 bp, Homo sapiens
ggatgctgaggaagcgtcggtgacatgaagaggggtggcgtgacctgtgga

SEQ ID NO: 56 - DNA, 2906 bp, Homo sapiens
gtcacctgtggatgctgaggaagtgctggtgacaggaagaggggtgccgtgacctgtggacactgaggaagcgtcggtgacaggaagagaggtggtgtgacctgaggatgctgaggaagggatggtgacaggaagaggcgtggtgtcacctgtggatactgaggaaaggctggtgacaggaagaggggtggcctgacctgtggatgcagaggaagtgtcggtgacaggaagaggcgtggtgtcacctgtggatactgaggaaaggctggtgagaggaagaggggtggcgtgaccggtggatgctgaggaagcatcggtgacaggaagagtgctggtgtcacctctggatgctgaggaagggctggtgacatgaagaggggtggcgtgacctgtggataatgaggaagcattggtgacaggaagaggggtggtgtcacctgtggatgctgaggaagtgctggtgacaggaacaggggtggcgtgacctgtggatgctgaggaagggctggtgacaggaagaggggtggcgtgacctgtagatactgaggaagtgctggtgacaggaagaggggtggcgtgacctgtggatactgaggaagtgtcggtgacaggaaggggggtggcgtgacctgtggatgctgaggaacggctggtgacaggaagagaggtggcgtgacctgtggatactgaggaagtgtcggtgacaggaagaggggtggtgtcacctgtggatgctgaggaagtgctggtgacaggaagaggggtggcatgtcctgtggatgccgaggaaacgtcggtgacaggaagacgggtggtgtcatctgtggaagctgaggaagtgtcggtgacaggaagaggggtggcgtgacgtgtggatgctgaggaagtgtcggtgacaggaagaggggtggtgtcacctgtggaagctgaggaaaggccggtaacaggaagaggggtggcgtgacctgtggatgctgaggaagggctagtgacaggaagaggcatggtgtcacctgtggatactgaggaagggatggtgacaggaagaggggtggcgtgacctgtggatgctgaggaagcgtcggtgacaggaagaggggtggtgtcacctgtggatactgaggaaaggctggtgacaggaagaggggtggcctgacctgtggatgctgaggaagtgtcggtgacaggaagaggggtggtgtcacctgtggatgctgaggaagcgtcggtgacaggaagaggcgtggcgtgacctgtggacactgaggaagcgtcggtgacaagaagaggggtggcgtgacctgtggatgctgaggaagtgctggtgacaggaagaggggtgacgtgacctgtggatgctgaggaagggctggtgacatgaagaggggtgacgtgacctgtagatactgaggaagtgctggtgacaggaagaggggtggtgtgacctgaggatgatgaggaagggatggtgacaggaagaggggtggcctgacctgtggatgctgaggaagtgtccgtgacaggaagacgggtggtgtcacctgtggatgctgaggaagtgtcggtgacaggaagaggggtggcgtgacctgtggatactgaggaagcgtcggtgacaagaagaggggtggtgtcacctgtggatactgaggaaaggctggtgacaggaagaggggtggcctgacctgtggatgctgaggaagtgtccgtgacaggaagacgggtggtgtcacctgtggatgctgaggaagtgtcggtgacaggaagaggggtggcgtgacctgtggatactgaggaagcgtcggtgacaagaagaggggtggtgtcacctgtggatactgaggaaaggctggtgacaggaagaggggtggcctgacctgtggatgccgaggaagcgtcggtgacaggaagaggggtggtgtcacctgtggatactgaggaaaggctggtgacaggaagaggcgtggcgtgaccggtggatactgaggaagtgtcggtgacaggaagaggggtggcgtgaccggtggatgctgaggaagcgccggtgacaggaagagtgctggtgtcacctgtggatgctgaggaagggatggtgacatgaagaggggtggtgtgacctgtagatgctgaggaagggctggtgacaggaagaggggtggtgtcacctgtggatgctgaggaagtgtcggtgacaggaagaggggtggtgtgacctgtagatgctgaggaagtgctggtgacaggaacaggggtggcgtgaccggtggatgctgaggaagtgctggtgacaggaagaggggtggcgtgacctgtggatgctgaggaagggctagtgacaggaagaggcatggtgtcacctgtggatactgaggaagtgttggtgacaggaagaggggtggcctgacctgtggatgccgaggaaatgtcggtgacaggaagacgggtggtgtcacctgtggaagctgaggaaaggccggtgacaggaagaggggtggcgtgacctgtggatactgaggaagtgtcggtgacaggcacaggggtggtgtcacctgtggatgctgaggaagggctggtgacatgaagaggggtggcgtgacctgtggatgctgaggaagcgtcggtgacaagaagaggagtggcgtgacctgtggatactgaggaagtctcggtgacaagaagaggggtggtgtcacctgtggatgatgaggaagtgtcggtgacaggaagagaggtggtgtcacctgtgtatgctgaggaagtgtcggtgacaggaagagaggtggtgtcacctgtggatgctgaggaagtgtcggtgacaggaagagaggtggcatgaccggtggatgctgaggaagggctagtgacaggaagaggcgtggtgtcacctgtggatactgaggaaaggc

SEQ ID NO: 57 - DNA, 1460 bp, Homo sapiens
agtgacaggaagaggcatggtgtcacctgtggatgctgaggaaaggctggtgacaggaagaggggtggcgtgacctgtggatgctgaggaagggctggtgacatgaagaggagtgacgtgacctgtggatgctgaggaagtgctagtgacaggaagaggcgtggtgtcacctgtggatactgaggaagtgtcggtgacaagaagagaggtggcctgacctgtggatgctgaggaagtgtcggtgacaggaagaggggtggtgtgacctgtggatgctgcggaagggatggttacaggaagagaggtggcgtgatctgtggacactgaggaagcgtcggtgacaggaagaggggtggcgtgtcctgtggatgctgaggaagtgtcggtgacaagaagaggggtggcgtgacctgtggatgctgaggaagggctagtgacaggaagaggcgtggtgtcacctgtggatactgaggaaaggctggtgacaggaagaggggtggcctgacctgtggatgctgaggaagcatcagtgacatgaagaggggtggtgtgacctgtggatactgaggaagcgtcggtgacaggaagaggggtggtgtcacctgtggatgctgaggaagggctagtgacaggaagaggcatggtgtcacctgtggatgctgaggaaaggctggtgacaggaagaggggtggcgtgacctgtggatgctgaggaagggctggtgacatgaagaggagtgacgtgacctgtggatgctgaggaagtgctagtgacaggaagaggcgtggtgtcacctgtggatactgaggaagtgtcggtgacaagaagagaggtggcctgacctgtggatgctgaggaagtgtcggtgacaggaagaggggtggtgtgacctgtggatgctgcggaagggatggttacaggaagagaggtggcgtgatctgtggacactgaggaagcgtcggtgacaggaagaggggtggcgtgtcctgtggatgctgaggaagtgtcggtgacaagaagaggggtggcgtgacctgtggatgctgaggaagggctagtgacaggaagaggcgtggtgtcacctgtggatactgaggaaaggctggtgacaggaagaggggtggcctgacctgtggatgctgaggaagcatcagtgacatgaagaggggtggtgtgacctgtggatactgaggaagtgtcggtgacaggaagagaggtggcgtgacctgtggatgctgaggaagtgtcggtgacaggaagaggggtggtgtgacctgtggatactgaggaagtgtcggtgacaggaagagaggtggcgtgacctgtggatgctgaggaagtgtcagtgacaggaagaggggtggtgtcacctgtggatgctgaggaaaggctggtgacaggaagaggggtgacgtgacctgtggatgctgaggaagtgtcagtgacaggaagaggggtgc